(19) United States
(12) Patent Application Publication
(10) Pub. No.: US 2026/0285921 A1
(43) Pub. Date: Sep. 24, 2026

(54) NOVEL PORE MONOMERS AND PORES

(71) Applicants: Oxford Nanopore Technologies PLC, Oxford (GB); National Institute of Chemistry, Ljubljana (SI)

(72) Inventors: Elizabeth Jayne Wallace, Oxford (GB); Michael Robert Jordan, Oxford (GB); Lakmal Nishantha Jayasinghe, Oxford (GB); Tomaz Svigelj, Ljubljana (SI); Marija Srnko, Ljubljana (SI); Gasper Solinc, Ljubljana (SI); Ana Crnkovic, Ljubljana (SI); Marjetka Podobnik, Ljubljana (SI); Gregor Anderluh, Ljubljana (SI)

(73) Assignees: Oxford Nanopore Technologies PLC, Oxford (GB); National Institute of Chemistry, Ljubljana (SI)

(21) Appl. No.: 19/474,204

(22) PCT Filed: Apr. 15, 2024
(86) PCT No.: PCT/EP2024/060202

§ 371 Date: Oct. 10, 2025

(30) Foreign Application Priority Data
Apr. 14, 2023 (GB) 2305524.7

(51) Int. Cl. C07K 14/435; G01N 33/487; G01N 33/68

(52) U.S. Cl. CPC C07K 14/43595; G01N 33/48721; G01N 33/68; G01N 2333/43595

(57) ABSTRACT

The present invention relates to novel actinoporin monomers, actinoporin pores formed from the monomers and their uses in analyte detection and characterisation.

### RELATED APPLICATIONS

This application is a national stage filing under 35 U.S.C. § 371 of international application number PCT/EP2024/060202, filed Apr. 15, 2024, which claims the benefit of United Kingdom application number GB 2305524.7, filed Apr. 14, 2023, each of which is herein incorporated by reference in its entirety.

### REFERENCE TO AN ELECTRONIC SEQUENCE LISTING

The contents of the electronic sequence listing (O036670176US00-SEQ-KZM.xml; Size: 3,178 bytes; and Date of Creation: Sep. 29, 2025) are herein incorporated by reference in its entirety.

### TECHNICAL FIELD

The present invention relates to novel actinoporin monomers, actinoporin pores formed from the monomers and their uses in analyte detection and characterisation.

### BACKGROUND

Nanopore sensing is an approach to analyte detection and characterisation that relies on the observation of individual binding or interaction events between the analyte molecules and an ion conducting channel. Nanopore sensors can be created by placing a single pore of nanometre dimensions in an electrically insulating membrane and measuring voltage-driven ion currents through the pore in the presence of analyte molecules. The presence of an analyte inside or near the nanopore will alter the ionic flow through the pore, resulting in altered ionic or electric currents being measured over the channel. The identity of an analyte is revealed through its distinctive current signature, notably the duration and extent of current blockades and the variance of current levels during its interaction time with the pore. Analytes can be organic and inorganic small molecules as well as various biological or synthetic macromolecules and polymers including polynucleotides, polypeptides, and polysaccharides. Nanopore sensing can reveal the identity and perform single molecule counting of the sensed analytes but can also provide information on the analyte composition such as nucleotide, amino acid, or glycan sequence, as well as the presence of base, amino acid, or glycan modifications such as methylation and acylation, phosphorylation, hydroxylation, oxidation, reduction, glycosylation, decarboxylation, deamination and more. Nanopore sensing has the potential to allow rapid and cheap polynucleotide sequencing, providing single molecule sequence reads of polynucleotides of tens to tens of thousands bases length.

Two of the essential components of polymer characterization using nanopore sensing are (1) the control of polymer movement through the pore and (2) the discrimination of the composing building blocks as the polymer is moved through the pore. During nanopore sensing, the narrowest part of the pore forms the constriction, the most discriminating part of the nanopore with respect to the current signatures as a function of the passing analyte. There is a need for new nanopores for use in analyte characterisation.

Orbicella faveolata, or mountainous star coral as it is commonly known, contains an actinoporin whose sequence is not known.

### SUMMARY OF THE INVENTION

The invention relates to modified actinoporin monomers and actinoporin pores comprising the monomers. The actinoporin monomers and the actinoporin pores comprising them are modified to facilitate the characterisation of target analytes, especially target proteins and polypeptides, and to facilitate insertion into an artificial membrane, and to facilitate oligomerisation.

The inventors have identified the sequence of the Orbicella faveolata actinoporin monomer. This is shown in SEQ ID NO: 1 (without the signal peptide). The inventors have also surprisingly demonstrated that N-terminal deletions are required for insertion of the actinoporin monomers into artificial membranes and the insertion of functional actinoporin pores in artificial membranes. The inventors have also surprisingly demonstrated that particular substitutions, especially D203R (using the residue numbering in SEQ ID NO: 1), in the sequence of the actinoporin monomer affect the number of monomers that form actinoporin pores in artificial membranes. The number of such monomers that form actinoporin pores can be further increased by C-terminal deletions. Finally, the inventors have surprisingly demonstrated modified actinoporin monomers of the invention may be used to form actinoporin pores that are capable of characterising analytes, especially proteins or polypeptides. The invention therefore provides an actinoporin monomer comprising a variant of SEQ ID NO: 1 having at least about 60% identity to the sequence of SEQ ID NO: 1 over its entire length. The invention also provides an actinoporin monomer comprising a variant of SEQ ID NO: 2 having at least about 54% identity to the sequence of SEQ ID NO: 2 over its entire length.

The invention also provides:
a construct comprising two or more covalently attached actinoporin monomers of the invention,
a polynucleotide which encodes actinoporin monomer of the invention or a construct of the invention,
an actinoporin pore comprising at least one actinoporin monomer of the invention or at least one construct of the invention,
an artificial membrane comprising an actinoporin pore derived from Orbicella faveolata,
a method of determining the presence, absence or one or more characteristics of a target analyte, comprising (a) contacting the target analyte with an actinoporin pore of the invention or a membrane of the invention and (b) taking one or more measurements as the target analyte moves with respect to the actinoporin pore and thereby determining the presence, absence or one or more characteristics of the target analyte,
use of an actinoporin pore of the invention or a membrane of the invention for determining the presence, absence or one or more characteristics of a target analyte,
a kit for characterising a target analyte comprising (a) an actinoporin monomer of the invention, a construct of the invention or an actinoporin pore of the invention and (b) the components of a membrane and/or an analyte binding protein,
an apparatus for characterising a target analyte in a sample, comprising (a) a plurality of actinoporin monomers of the invention, a plurality of constructs of the invention or a plurality of actinoporin pores of the invention and (b) a plurality of analyte binding proteins,
an array comprising a plurality of membranes of the invention,
a system comprising (a) a membrane of the invention or an array of the invention, (b) means for applying a potential across the membrane(s) and (c) means for detecting electrical or optical signals across the membrane(s),
an apparatus produced by a method comprising (a) obtaining an actinoporin pore derived from Orbicella faveolata and (b) and contacting the actinoporin pore with an in vitro membrane such that the actinoporin pore is inserted in the in vitro membrane, and
an apparatus comprising an actinoporin pore derived from Orbicella faveolata inserted into an in vitro membrane.

### DESCRIPTION OF THE FIGURES

FIG. 1: N-terminal deletions (gel). SDS page of wtFav monomer (theoretical MW 28 kDa) and its N-terminal deletion mutants. Soluble monomers were expressed in E. coli with N-terminal HisTag (removed by TEV protease) and purified by affinity chromatography.

FIG. 2: N-terminal deletions (electrophysical measurements). Current (I) versus voltage (V) curves (I/V) curves obtained after insertion of Fav N-terminal deletion mutants into MinION membranes and applied alternating voltage from 0 to ±120 mV.

FIG. 3: 8mer to 9mer transition (gel). Native-PAGE of Fav pores ΔN75 Fav, ΔN75 D203R Fav, ΔN75 D215N Fav, ΔN76 D203R Fav, ΔN75 D203R D215N Fav, and RN-Fav obtained by incubation with 50 mM DOPC:SM (molar ratio 1:1) large unilamellar vesicles (LUVs). LUVs were solubilised with Triton X-100 prior to loading.

FIG. 4: 8mer to 9mer transition (Cryo-EM 2D classes). 2D classes obtained after analysing Cryo-EM micrographs of detergent solubilised wtFav and RN-Fav pores revealing a presence of mostly octameric pores for wtFav and octameric and nonameric pores (ratio 1:1) for RN-Fav pores.

FIG. 5: 8mer to 9mer transition (structures). 3D volumes (top and side views) of RN-Fav octameric and nonameric pores. Each protomer in the pore is in different shade.

FIG. 6: Shift of the the octamer/nonamer ratio towards nonamers. Native PAGE of RN-Fav and ΔC4-RN Fav pores obtained by incubating corresponding monomers with 50 mM DOPC:SM (molar ratio 1:1) LUVs and LUVs were solubilised with Triton X-100. Arrows indicating the bands corresponding to octameric and nonameric pores. Data are showing that modifications at Fav C-terminus shifts the octamer/nonamer ratio toward nonamers.

FIG. 7: Shift of the the octamer/nonamer ratio towards nonamers. 2D classes obtained after analysing Cryo-EM micrographs of detergent solubilised wtFav and ΔC4-RN-Fav pores revealing a presence of mostly octameric pores for wtFav and additional appearance of decameric pores (nonamers:decamers ratio 19:1) for ΔC5-RN Fav pores. 2D classes of potential decamers are marked with red rectangle (top two on the far right).

FIG. 8: RN-Fav (MinION). MinION experiments of RN-Fav pores. A: Typical I/V curves obtained after applied alternating voltage from 0 to ±120 mV (lower panel). B: Electrical current histogram for all active channels for octameric (N=478) and nonameric (N=168) RN-Fav. C: Averaged I/V curve (three independent flow cells) for channels with a single pore and D: corresponding S2N ratio.

FIG. 9: RN1-Fav (MinION). MinION experiments of RN1-Fav. A: Typical I/V curves obtained after applied alternating voltage from 0 to ±120 mV (lower panel). B: Electrical current histogram for all active channels for octameric (N=485) and nonameric (N=100) RN1-Fav. C: Averaged I/V curve (three independent flow cells) for channels with a single pore and D: corresponding signal-to-noise (S2N) ratio.

FIG. 10: Positively charged peptide detection. Short lived electrical current blockages observed after addition of R14 peptides to octa- (top traces) or nonameric (bottom traces) RN1-Fav pores inserted into MinION membranes.

FIG. 11: Histone detection. Normalised current versus time traces and normalised current histograms for RN1-Fav pores (MinION membranes), capturing different histone variants (2 μM).

FIG. 12: Histone detection. Histone variants, detected by RN1-Fav pores inserted into MinION membranes, could be distinguished based on averaged currents and noise at +50 mV. Circles represent mean normalised currents vs. its noise with standard deviations for membranes with a single pore (N) within a single flow cell.

FIG. 13: Covalently attaching monomers (addition of cysteines). Cysteines introduced at amino acid positions 84 and 85 (corresponding to SEQ ID NO: 1) cause the formation of disulphide (S—S) bond ring at the pore transmembrane region. A: SDS Page of CC-Fav pores in the absence and presence of a reducing agent, causing the reduction of the disulphide (S—S) bond and disappearance of higher oligomeric complexes (the 160 kDa band corresponds to eight attached protomers). B: Top and side view of wtFav and CC-Fav 3D structures. Red arrows pointing to S—S bond ring.

FIG. 14: Structural characterization of RN1-Fav pores. a) A schematic representation of the proteins used in this study. The positions of the mutations are indicated by yellow stars. See the alignment in Figure S1 for details. b) Native PAGE gel of the pores used in this study. The position of octamers and nonamers is indicated. c) Overall architecture of the octameric RN1-Fav pore model solved by cryo-electron microscopy. Black arrows indicate the overall dimensions of the pore. The putative membrane region is shown as a grey rectangle. Red and green rectangles are shown enlarged in panels d and e. d) The magnification of the pore structure around residue R203, which shows formation of H-bonds with neighboring side chains of the other protomer. Side chains of the indicated amino acid residues are shown in ball and stick representation. Blue balls, nitrogen; red balls, oxygen; green dotted line, potential H-bonds. e) The magnification of the helix region with all mutated residues in RN1-Fav is shown in ball and stick representation. f and g) Electrostatic potential of the FraC, wtFav and RN1-Fav pores, top view (f) and cross-sections (g), with the color gradient from negative (red) to positive (blue) potential. The pore constriction is highlighted with arrows and the diameters indicated (measured from Van der Waals surface to Van der Waals surface). The dimensions of the pore lumen in the cap region is also indicated for the octamer of RN1-Fav.

FIG. 15: High-throughput electrophysical characterization of Fav pores. a) Representative current traces recorded with the MinION system after applying a 10 mV step voltage ramp from 0 mV to ±120 mV. All traces were filtered with a 5 kHz low-pass filter. The black straight line marks 0 pA or 0 mV. Black, data for the octameric pore; red, data for the nonameric pore. b) Current histogram of the open pore at −50 mV with Gaussian fits where applicable. The number of pores (n), included in analysis also in other panels, is given for each variant. We also report the mean current±standard deviation for all RN-Fav and RN1-Fav pores resulting from the fits. c) An averaged I/V curve for all detected single pores per MinION flow cell recorded with the same ramping protocol as in (a), with the corresponding standard deviations. d) Box plot showing the signal-to-noise ratio at −50 mV, calculated for all single pores. The difference was tested with the Two Sample t-test. **p<0.01, *p<0.05, n.s. stands for no statistical difference (p>0.05). e) Mean signal-to-noise ratio±standard deviation for RN1-Fav octamer pores over the entire voltage ramp, calculated for the pores included in other panels.

FIG. 16: Label-free differentiation of human histone protein variants by bulk analysis. a) A schematic model of full-length histone detection (blue) with an octameric RN1-Fav pore. The histones were added from the cis side of the membrane. b) Amino acid sequence of the unstructured N-terminal histone tails of the histones used in this study. Amino acids with positively charged side chains are colored blue and post-translationally modified amino acids are colored red. c) 3D models of the H3.1 (blue) and H4 (black) core domain (PDB 2CV5 [36]) with N- and C-termini labelled. d) Representative one-minute current traces recorded at −50 mV. The grey traces represent control measurements of the same pore in the absence of histones, while the colored traces correspond to current traces obtained after the addition of 2 μM histone as indicated. e) Discrimination of histone variants based on relative blockade current and noise for the 60 s long current traces of individual pores recorded simultaneously with a single flow cell (each circle represents a MinION flow cell and the corresponding number of individual pores included in the analysis). The standard error of the normalized blockade current and the noise are shown as grey error bars.

FIG. 17: Histone discrimination based on blockade analysis. a) One-minute current trace recorded at −50 mV in the presence of 2 μM histones. The colored sections of the trace represent the detected current blockades for H4 (black) and H3Cit (green). The histograms on the right side of each trace correspond to the current trace on the left side. The bars representing the detected blockade currents are shown in the corresponding colors. b) 3D scatter plot of normalized blockade noise, amplitude and dwell time for the blockades extracted from three independent flow cells in the presence of 2 M histone (the data for each flow cell are shown as circles with different black (for H4) or green (H3Cit) intensity. For H4 data, 534, 260 and 460 blockades were extracted from flow cells with 179, 39 and 120 single pores, respectively. For H3Cit, 110, 386 and 42 blockades were extracted from 40, 280 and 25 pores, respectively. c and d) One-minute current traces (low-pass filtered with a 1000 Hz cut-off) after addition of 0, 0.5, 1, 2 and 4 μM histone H4 (c) or histone H3Cit (d). e) Linear fit to the concentration-dependent blockade frequency (1/TON). The data represent an average±standard error calculated for 3-6 independent flow cells.

FIG. 18: Machine learning supports the discrimination of histones in a mixture. a) A workflow diagram of blockade classification by using Orange. [39] For training the model, the amplitude, noise and dwell time of the blockades extracted from the traces where only H4 or H3Cit was added were used as input data for future matrix generation. The model with a classification accuracy (CA) of 0.960 was generated using the kNN algorithm and cross-validating 10% of the data. b) The confusion matrix generated with the data from the test set (random 20% of the input data) resulted in an accuracy of 91.3% for histone H3Cit and 97.7% for histone H4. c) Blockades (bold part of the traces) whose parameters were extracted from a one-minute trace where a 1:0, 3:1; 1:1, 1:3 and 0:1 H4 and H3Cit ratio (at a total concentration of 2 μM) were used for classification based on the previously learnt model. The colors correspond to the blockades recognized as H3Cit (green) or H4 (black). d) The experimentally determined ratio of histones in the mixture is based on the number of blockades classified as H4 (black) or H3Cit (green) out of a total of 1238,158,381,176 and 347 input blockades. e) Model-based estimate of the proportion of H4 in the mixture (black circles with linear fit) compared to the predicted theoretical values (open circles). The data represent an average±standard error of three independent flow cells.

FIG. 19: Amino acid sequence alignment of wtFav, ΔN67Fav, RN-Fav, RN1-Fav that were used in experiments and FraC colored by conservation. Mutations introduced to wtFav in different constructs are colored green. N-terminal residues of constructs ΔN53Fav, ΔN63Fav, ΔN67Fav, ΔN73Fav and ΔN75Fav are marked in red on the wtFav sequence.

FIG. 20: Insertion of ΔN-Fav constructs into MinION flow cells. a) Bar plot representing the number of specific Fav pores with truncated N-terminus inserted into MinION flow cell (black dots). Median values calculated for three flow cells (7 in case of ΔN67Fav) are marked in red. b) Representative current traces obtained by MinION when applying alternating voltage protocol from 0 my to ±120 mV (except for ΔN53Fav, where the voltage increased from 0 mV to ±90 mV). All the traces were filtered using MATLAB lowpass filter with 1000 Hz cut-off. c) Histograms of open pore current for ΔN53, ΔN63, ΔN67, ΔN73, ΔN75Fav were extracted from the I/V curve measurements at −90 mV.

FIG. 21: Data analysis workflow. a) Data analysis workflow for solubilized octameric and nonameric RN1-Fav pores from micrographs to b) final sharpened volumes colored by local resolution estimate. Both structures (octameric, nonameric pore) were resolved from the same dataset following the same protocol. Arrows indicate the movement of particles between different steps or iterations. Throughout the steps, the volume of the octameric pore is shown in grey and that of the nonameric pore is shown in blue. Cyan and wheat are the two junk/decoy volumes. When separated the analysis of octameric and nonameric pores is indicated by green and yellow rectangles, respectively. Additional details on data analysis are provided in the cryo-EM data processing section of Materials and Methods of Example 2

FIG. 22: a) RN1-Fav octameric pore model built into the cryo-EM density map. Individual protomers are coloured yellow and blue with lipids coloured green. Regions of the map where RN1-fav mutations are located are highlighted and shown in panels b and c. b) Region of the map around residue R203. c) Region of the cryo-EM density map of the transmembrane α-helix with highlighted residues that are different from wtFav. d) Side view of the RN1-Fav octameric pore, protomers are in alternating colors (sky blue and yellow). e) Side view of the RN1-Fav nonameric pore, protomers are in alternating colors (sky blue, orange, and yellow). f) Top view of aligned octameric wtFav (PDB-ID 9EYM, navy blue and violet) and octameric RN1-Fav pore (yellow and cyan), RMSD=0.51 (PDBeFold) [6] over all C-α atoms.

FIG. 23: Separation of octameric and nonameric RN-Fav and RN1-Fav pores by ion-exchange chromatography. a) Ion-exchange chromatograms for RN-Fav pores (left) and RN1-Fav pores (right) eluted with increasing concentration of 2 M NaCl (yellow line). Fractions numbered from 1 to 6 were analyzed by Native PAGE as indicated in (b) and (c) for RN-Fav and RN1-Fav pores, respectively.

FIG. 24: Histone H4 capturing at different voltages. a) Alternating voltage ramp from 0 mV to +/−100 mV was applied in the presence of 1 μM histone H4. Light blue star marks the time point when the membrane is disrupted and pore destroyed. An example of current traces for four independent membranes in C18 buffer (gray) and in the presence of 1 μM histone H4 (black). b) Zoom in of current traces at voltages from −40 mV to −90 mV, enlarged part of the trace from panel (a) in the green box. Blue asterisks mark discrete blockades shorter than 5 s and red asterisks indicate long-lasting blockades that often last until the change of the voltage polarity. c) Mean dwell times with standard errors for all detected events (corresponding numbers are shown in the graph). d) Zoom of the purple box from the panel a) showing high current noise at −100 mV that disabled us from determining discrete blockades.

FIG. 25: Histone H3.1 capturing at different voltages. Alternating voltage ramp from 0 mV to +/−100 mV was applied in the presence of 1 μM histone H3.1. a) An example of current traces for four independent membranes in C18 buffer (gray) and histone H3.1 (black). Light blue star marks the time point when the membrane is disrupted and pore destroyed. b) Zoom in of current traces at voltages from −40 mV to −80 mV, for part of the trace from panel a) in a green box. Blue asterisks mark discrete blockades shorter than 5 s and red asterisks indicate long-lasting blockades that often last until the change of the voltage polarity. c) Mean dwell times with standard errors for all detected events (corresponding numbers are shown in the graph).

FIG. 26: Open pore current restoration at positive voltage polarity. For histone detection we used voltage protocol starting at 0 mV, changing for 60 s to −50 mV and at the end increasing to +50 mV (yellow line). The current signal corresponding to the control measurement (no added histones) is shown in gray, while the signal in the presence of 2 M histones H3.1 and H3K23ac is colored black. Red arrows mark the voltage polarity change and consequent open pore current restoration.

FIG. 27: MinION measurement repeatability for histone H4. Data for three independent flow cells with inserted octameric RN1-Fav pores (single pore per membrane). Each row represents one-minute normalized current trace recorded at −50 my in the absence (gray) and presence of 2 μM histone H4.

FIG. 28: MinION measurement repeatability for histone H3.1. Data for three independent flow cells with inserted octameric RN1-Fav pores (single pore per membrane). Each row represents one-minute normalized current trace recorded at −50 my in the absence (gray) and presence of 2 μM histone H3.1.

FIG. 29: MinION measurement repeatability for histone H3Cit. Data for three independent flow cells with inserted octameric RN1-Fav pores (single pore per membrane). Each row represents one-minute normalized current trace recorded at −50 my in the absence (gray) and presence of 2 μM histone H3Cit.

FIG. 30: MinION measurement repeatability for histone H3K9ac. Data for three independent flow cells with inserted octameric RN1-Fav pores (single pore per membrane). Each row represents one-minute normalized current trace recorded at −50 my in the absence (gray) and presence of 2 μM histone H3K9ac.

FIG. 31: MinION measurement repeatability for histone H3K23ac. Data for three independent flow cells with inserted octameric RN1-Fav pores (single pore per membrane). Each row represents one-minute normalized current trace recorded at −50 my in the absence (gray) and presence of 2 μM histone H3K23ac.

FIG. 32: Event analysis workflow. After pore insertion into MinION membranes we applied a voltage protocol starting at 0 mV, then dropping to −50 mV and then rising to +50 mV. Histone capturing occurred at negative applied voltages. Prior to histone addition to the cis side of the membrane, we recorded open pore current in the absence of histones (C18 buffer only, control measurement). By in-house MATLAB script we extracted all the active membranes and constructed open pore current (−50 mV) histogram for all the MinION membranes that stayed active (have a pore signal) after histone addition. By multiple Gaussian fit we extracted data for the membranes with a single pore inserted. In the next step we applied wavelet-based filter and perform current normalization by dividing open pore current (I0) with averaged blockade current of specific trace (Ik0). Blockades were extracted by our MATLAB script with threshold-based method and characterized by their duration (dwell time), current amplitude, mean blockade current and its standard deviation. Additional details on data analysis are provided in the MinION data analysis section of Methods in Example 2.

FIG. 33: The structure of the Fav pore formed on DOPC:SM (1:1) membranes. a, A top and a side view of cryo-electron microscopy map of octameric Fav pore prepared on DOPC:SM vesicles. Parts of the map corresponding to individual pore protomers are coloured differently, regions of the map corresponding to well-defined lipids are shown in orange. b, Cartoon representation of the pore. The cap region and trans-membrane (TM) helical bundle are highlighted. c, Electrostatic potential of the surface of the pore. The colour gradient from red to blue represents negative to positive electrostatic potentials. Fenestrations between TM helices, residue E91 (E88 in SEQ ID NO: 1) and the extension at the C-terminus (green) are highlighted. d, Six unique lipid positions identified from the pore structure. The residue numbering in FIG. 33d corresponds to SEQ ID NO: 1.

FIG. 34: The structure of Fav pore prepared on POPG:SM:CHOL (1:1:1) vesicles. a, A side view of cryo-EM map of octameric Fav pore. Parts of the map corresponding to individual pore protomers are coloured differently, and regions corresponding to well-defined lipids are shown in orange. b, Surface and cartoon/sticks representation of four cholesterol (CH) molecules. Green dashed lines show hydrogen bonds between cholesterol and protomer. c, Comparison of discrete ion current steps, corresponding to pore insertion induced by the addition of a monomeric Fav protein at −50 mV (top). d, Normalized current noise for membranes in the absence or presence of cholesterol. n=12 and 17, respectively. e,f A look at a surface representation of a promoter with parts of the map corresponding to lipids associated with it at two different angles. The lipids are coloured according to the assigned function. Bottom-up view of a protomer with all visible lipids. g, Surface and cartoon/sticks representation of phospholipids resolved in the pore structure, highlighting bridging lipids. h, A schematic representation of lipids and proteins observed in cryo-EM structure with emphasis on bridging lipids and L10. Hydrogen bonds between lipids are denoted by green lines, hydrogen bonds between lipids and proteins are denoted by a line with the circle. Hydrophobic interactions between lipids are shown as magenta lines. The residue numbering in this Figure corresponds to SEQ ID NO: 1.

FIG. 35: Structural and dynamic properties of membrane lipids from molecular dynamics simulations. a,b, Schematic bead representation of lipid molecules, which are divided into successive coordination shells in upper (a) and lower (b) membrane leaflet. Lipid molecules colored red represent the shell of lipids in the “strong” contact with the protein. Each next successive shell is alternatively colored green and blue with bulk lipids in gray. The remaining predefined lipids in the upper layer are shown gray. c, Correlation between displacements of the neighboring lipid molecules belonging to successive shells: current (λ) versus previous shell (λ−1) of lipids for the upper (blue circles) and lower leaflet (black circles) according to equation (1). Full line connects values obtained for the cholesterol containing system with 112 predefined lipid poses and dashed line connects values for the cholesterol free system with 48 predefined lipid poses. d, Lateral diffusion constants of the lipid molecules in successive lipid shells around the pore in the upper or lower leaflet. Colors and line types are the same as in c. The diffusion constants obtained for the pure membrane systems are given together with the error range. Blue, POPC:SM:CHOL membrane system; green, POPC:SM membrane system. Layer 0 refers to diffusion constant due to the nearest protein monomer. e, Schematic representation of different types of lipids and interactions derived from transmembrane pore complex structure and molecular dynamics simulations.

FIG. 36: Structure of Fav and pore preparation. a, Amino acid sequence alignment of Fav with actinoporins. Identical residues are marked with red background and chemically similar residues are displayed in the red font. SEQ ID NO: 1 starts at the S at position 4 of the Fav sequence. b, Superposition of crystal structures of Fav (this work, PDB-ID 9EYM, orange) and actinoporin FraC (PDB-ID 3LIM, grey). N- and C-termini and, various structural elements are labelled. The disulphide bond is labelled yellow. c, A structural detail with a density map of the region L179-V185 (L176-V182 in SEQ ID NO: 1). 2mFoDFc electron density is contoured at 1 σ (blue) and mFo-DFc electron density at +3.0 σ (green) and at −3.0 σ (red). d, Cryo-EM micrograph of DOPC:SM (1:1) large unilamellar vesicles (LUVs) incubated with monomeric Fav (top and 2D class averages of two different pore stoichiometries (bottom). e, Schematic representation of Fav pore preparation steps, from top to bottom: 1 preparation of protein-LUV mixture, 1 h incubation at 37° C., solubilization with 1% lauryl dimethylamine oxide, purification on ion exchange chromatography column, Native-PAGE of monomeric Fav (M) and purified Fav pore (P).

FIG. 37: Data analysis workflow for isolated Fav pore formed on large unilamellar vesicles composed of DOPC:SM (1:1). Additional details on data analysis are provided in Cryo-electron microscopy data processing section of Materials and methods

FIG. 38: Comparison of lipids previously described in fragaceatoxin C (FraC) pore (PDB-ID 4TSY) and lipids at equivalent positions in the structure of the Fav pore. a, Side-by-side comparison of individual protomers with lipids L1, L2, and L3. b, Fav protomer (blue) aligned to FraC protomer (gray) with corresponding lipids (orange for Fav and yellow for FraC).

FIG. 39: Lipids in the Fav pore structure prepared on liposomes composed of DOPC:SM (1:1). Maps with fitted models are shown for each lipid position (left), with an interaction diagram (right). Hydrogen bonds are represented with green dashed lines. Red lines represent hydrophobic interactions with protein residues and green with other lipids. The interaction diagrams were prepared by LigPlot+ (Laskowski, R. A. & Swindells, M. B. LigPlot+: multiple ligand-protein interaction diagrams for drug discovery. Journal of chemical information and modeling 51, 2778-2786, doi:10.1021/ci200227u (2011)). The residue numbering in this Figure corresponds to SEQ ID NO: 1.

FIG. 40: Interaction surface between two protomers in a pore. a, A position of lipids L1 and L6 between the protomers. b, A schematic representation of interaction surfaces between protomers A and B. Green represents the direct protomer-protomer surface and orange the indirect protomer-lipid-protomer interactions. b, Interaction surfaces as calculated by pdbePISA (Krissinel, E. & Henrick, K. Inference of macromolecular assemblies from crystalline state. J Mol Biol 372, 774-797, doi:10.1016/j.jmb.2007.05.022 (2007)). c, interaction diagram of the surface between two protomers. Green lines represent hydrogen bonds, while red and pink are hydrophobic interactions. All residue numbers in this Figure show the position in SEQ ID NO: 1+3 (e.g., L139 in the Figure is L136 in SEQ ID NO: 1).

FIG. 41: Alternative lipid chain positions of lipid L2 and L4. a, bottom-up view at the pore (white) and lipids with a highlighted void underneath the cap region. The denoted area is shown enlarged on the right. b, Two sets of lipids at positions L2 and L4. c, skeletal models of the lipids fit into corresponding cryo-EM maps.

FIG. 42: Data analysis workflow for isolated Fav pore formed on large unilamellar vesicles composed of POPG:SM:cholesterol (1:1:1). Additional details on data analysis are provided in Cryo-electron microscopy data processing section of Materials and methods.

FIG. 43: Comparison of pores formed on large unilamellar vesicles composed of DOPC:SM (1:1) or POPG:SM:CHOL (1:1:1). Cryo-EM maps are coloured by protomers, and regions corresponding to lipids are shown in orange.

FIG. 44: Cholesterol molecules in the Fav pore structure formed on large unilamellar vesicles composed of POPG:SM:CHOL (1:1:1). Maps with fitted models are shown for each cholesterol position (left), with an interaction diagram (right). Hydrogen bonds are represented with green dashed lines. Red lines represent hydrophobic interactions with protein residues and green with other lipids. The interaction diagrams were prepared by LigPlot+ (Laskowski, R. A. & Swindells, M. B., supra). The residue numbering in this Figure corresponds to SEQ ID NO: 1.

FIG. 45: Comparison of pore structures formed in different membranes. Comparison of pores formed on DOPC:SM (White) and POPG:SM:cholesterol (Blue) membranes. a, side-by-side comparison of the two pores. b, top view of aligned pores and c, aligned individual protomers.

FIG. 46: Lipids 1-16 in the Fav pore structure prepared on large unilamellar vesicles composed of POPG:SM:CHOL (1:1:1). Maps with fitted models are shown for each lipid position (left), with an interaction diagram (right). Hydrogen bonds are represented with green dashed lines. Red lines represent hydrophobic interactions with protein residues and green with other lipids. The interaction diagrams were prepared by LigPlot+ (Laskowski, R. A. & Swindells, M. B., supra). The residue numbering in this Figure corresponds to SEQ ID NO: 1.

FIG. 47: Lipids L7-L11 in the Fav pore structure prepared on POPG:SM:CHOL (1:1:1) membranes. Maps with fitted models are shown for each lipid position (left), with an interaction diagram (right). Hydrogen bonds are represented with green dashed lines. Red lines represent hydrophobic interactions with protein residues and green with other lipids. The interaction diagrams were prepared by LigPlot+ (Laskowski, R. A. & Swindells, M. B., supra). The residue numbering in this Figure corresponds to SEQ ID NO: 1.

FIG. 48: Data analysis workflow for isolated Fav pore formed on large unilamellar vesicles composed of DOPC:SM:cholesterol (1:1:1). a, Data analysis workflow for Fav pore prepared on DOPC:SM:CHOL (1:1:1) vesicles. Additional details on data analysis are provided in Cryo-electron microscopy data processing section of Materials and methods. b, Cryo-EM map of the Fav pore. Regions corresponding to protomers are coloured blue, while regions corresponding to lipids are coloured by their assigned group. c, Cartoon representation of a single protomer with associated lipids at two orientations.

FIG. 49: Analysis of Fav pore formed on nanodiscs composed of DOPC:SM:cholesterol (1:1:1). a, Data analysis workflow for Fav pore prepared on DOPC:SM:CHOL (1:1:1) nanodiscs. Additional details on data analysis are provided in Cryo-electron microscopy data processing section of Materials and methods. b, Cryo-EM map of Fav pore prepared on nanodiscs. Regions corresponding to cholesterol are coloured red and sphingomyelin are orange.

FIG. 50: The position of the Fav pore in the membrane. a, Fav protomer with highlighted membrane binding loops 2 and 3. The angle at which the protomer is placed on the membrane is indicated. b, Fav pore (blue) after minimization embedded into a POPC:SM:cholesterol (1:1:1) membrane that was used for molecular modelling. Loops 2 and 3 of all protomers are shown in green, orange balls are phosphate groups of lipids and upper leaflet cholesterol molecules are shown in red. c, Cartoon representation of a protomer with highlighted loops 2 and 3, with lipids bound to it. d, The surface representation of the Fav protomer with additional lipids shown at two different angles.

FIG. 51: Comparison of soluble and protomer structures of Fav. a, Monomer in solution is shown in gray, a single protomer from the pore structure is shown in blue. Details of loops 2 (b) and 3 (c) are shown with side chains of amino acids presented as sticks. All residue numbers in this Figure show the position in SEQ ID NO: 1+3 (e.g., V160 in the Figure is V157 in SEQ ID NO: 1).

FIG. 52: Pore formation by Fav on large unilamellar vesicles containing ceramide phosphoethanolamine (CPE). a, Ion exchange chromatography of samples obtained by incubating Fav with 1: liposomes DOPC:SM:Cholesterol (1:1:1), 2: liposomes DOPC:CPE:Cholesterol (1:1:1) or 3: without liposomes and solubilised by 0.75% lauryl dimethylamine oxide and eluted by linear NaCl gradient (dashed line). b, Native PAGE gel of peak fractions 1-3 obtained after chromatography step as shown in a. c, Representative micrograph (top) of large unilamellar vesicles composed of DOPC:CPE:cholesterol after incubation with Fav. 2D class averages are shown below.

FIG. 53: Interactions of L1 with Fav protomers. a, Interactions between sphingomyelin headgroups that would be absent if ceramide phosphoethanolamine was at the same position. b, surface presentation of protomers around headgroup of L1.c,d, Comparison of interaction diagram of L1 (c) and potential interaction diagram of ceramide phosphoethanolamine (d) at position L1. The residue numbering in this Figure corresponds to SEQ ID NO: 1.

### DESCRIPTION OF THE SEQUENCE LISTING

SEQ ID NO: 1 shows the amino acid sequence of the actinoporin monomer from Orbicella faveolata without the signal peptide.

SEQ ID NO: 2 shows a fragment of SEQ ID NO: 1 lacking amino acids 1-76 in SEQ ID NO: 1.

### DETAILED DESCRIPTION

All publications, patents and patent applications cited herein, whether supra or infra, are hereby incorporated by reference in their entirety. All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the invention contained in the specification, the specification is intended to supersede and/or take precedence over any such contradictory material.

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

In addition, as used in this specification and the appended claims, the singular forms “a”, “an”, and “the” include plural referents unless the content clearly dictates otherwise. Thus, for example, reference to “a polynucleotide” includes two or more polynucleotides, reference to “a polynucleotide binding protein” includes two or more such proteins, reference to “a helicase” includes two or more helicases, reference to “a monomer” refers to two or more monomers, reference to “an actinoporin pore” includes two or more actinoporin pores and the like.

In all of the discussion herein, the standard one letter codes for amino acids are used. These are as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y) and valine (V). Standard substitution notation is also used, i.e., Q42R means that Q at position 42 is replaced with R.

In the paragraphs herein where different amino acids at a specific position are separated by the/symbol, the/symbol means “or”. For instance, D203R/K means D203R or D203K. In the paragraphs herein where different positions are separated by the/symbol, the/symbol means “and” such that D203/D215 is D203 and D215.

The general definitions in WO 2019/002893 are incorporated by reference herein in their entirety.

### Actinoporin Monomers Based on SEQ ID NO: 1

The invention provides an actinoporin monomer comprising a variant of SEQ ID NO: 1 having at least about 40% identity to the sequence of SEQ ID NO: 1 over its entire length. The variant preferably has at least about 45%, at least about 50%, at least about 55%, at least about 60%, at least about 65%, at least about 70%, at least about 75%, at least about 80%, at least about 85%, or at least about 90% identity to the sequence of SEQ ID NO: 1 over its entire length. The variant more preferably has at least about 95%, at least about 97%, at least about 98% or at least about 99% identity to the sequence of SEQ ID NO: 1 over its entire length.

Standard methods in the art may be used to determine identity. For example, the UWGCG Package provides the BESTFIT program which can be used to calculate identity, for example used on its default settings (Devereux et al (1984) Nucleic Acids Research 12, p 387-395). The PILEUP and BLAST algorithms can be used to calculate identity or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

The % identity of the variant to SEQ ID NO: 1 is measured over the entire length of SEQ ID NO: 1. SEQ ID NO: 1 is 259 amino acids long. Identity is typically determined by calculating the number of identical residues between the variant and SEQ ID NO: 1 using any of the methods above and dividing the number of identical residues by 259. For instance, if a variant has 105 identical amino acids to SEQ ID NO: 1, the variant has 105/259×100=40.54% identity to SEQ ID NO: 1 over its entire length.

The actinoporin monomers of the invention are typically capable of forming a pore or an actinoporin pore. This can be measured using routine methods, including any of those described in WO 2016/034591, WO 2017/149316, WO 2017/149317, WO 2017/149318, WO 2018/211241, and WO 2019/002893 (all incorporated by reference herein in their entirety) and in the Examples.

The variant preferably comprises one or more modifications which (a) improve the ability of an actinoporin pore formed from the monomer to interact with a target analyte, (b) alter the number of actinoporin monomers which form an actinoporin pore and/or (c) facilitate insertion of the actinoporin monomer or an actinoporin pore formed from the monomer into an artificial membrane. The variant may comprise (a), (b), (c), (a) and (b), (a) and (c), (b) and (c), or (a), (b) and (c). Each of (a)-(c) are described in more detail below. The one or more modifications are preferably one or more mutations. The one or more modifications or mutations are preferably one or more additions, one or more deletions, one or more substitutions, or any combination thereof. The one or more modifications or mutations are preferably one or more substitutions.

The actinoporin pore formed from monomer preferably comprises one or more of (a) a cap region, (b) a constriction region, and (c) a transmembrane alpha helical region, such as (a), (b), (c), (a) and (b), (a) and (c), (b) and (c), or (a), (b) and (c). The actinoporin monomer preferably includes one or more regions which contribute to these regions in the actinoporin pore. The actinoporin monomer more preferably comprises one or more regions which form one or more of (a)-(c), such as (a), (b), (c), (a) and (b), (a) and (c), (b) and (c), or (a), (b) and (c). The actinoporin pore formed by the monomer may have any structure but preferably has or comprises the structure of the wild type actinoporin pore. The protein structure of actinoporin pore defines a channel or hole that allows the translocation of molecules and ions from one side of the membrane to the other.

The “constriction”, “orifice”, “constriction region”, “channel constriction”, or “constriction site”, as used interchangeably herein, refers to an aperture defined by a luminal surface of a pore or actinoporin pore, which acts to allow the passage of ions and target analytes (including but not limited to polynucleotides or individual nucleotides) but not other non-target analytes through the pore or actinoporin pore channel. In some instances, for example when characterising protein or polypeptide target analytes, the constriction prevents the target analyte from passing through the pore. The constriction(s) are typically the narrowest aperture(s) within a pore or actinoporin pore or within the channel defined by the pore or actinoporin pore. The constriction(s) may serve to limit the passage of molecules through the pore. The size of the constriction is typically a key factor in determining suitability of a pore or actinoporin pore for analyte characterisation. If the constriction is too small, the molecule to be characterised will not be able to pass through. However, to achieve a maximal effect on ion flow through the channel, the constriction should not be too large. For example, the constriction should not be wider than the solvent-accessible transverse diameter of a target analyte. Ideally, any constriction should be as close as possible in diameter to the transverse diameter of the analyte passing through.

SEQ ID NO: 1 shows the sequence of the wild type actinoporin monomer derived from Orbicella faveolata as a mature protein. Residues V104 to M259 of SEQ ID NO: 1 form the cap region. Residues A80 to L89 of SEQ ID NO: 1 form the constriction region. Residues L66 to N103 or R77 to N103 of SEQ ID NO: 1 form the transmembrane alpha helical region. The constriction region is typically present in the transmembrane alpha helical region. Residues S1 to D65 form a N-terminal extension.

The surface of the actinoporin monomer of SEQ ID NO: 1 exposed to the pore lumen or channel comprises a high negative charge. A pore formed from actinoporin monomers based on SEQ ID NO: 1 comprises a high negative charge in its lumen or channel. This allows such pores to determine the presence, absence or one or more characteristics of a positively charged analyte, such as a positively charged polypeptide, as described in the Examples.

The actinoporin monomer comprises a variant of SEQ ID NO: 1. The variant monomer may also be referred to as a modified actinoporin monomer or a mutant actinoporin monomer. The modifications, or mutations, in the variant include but are not limited to any one or more of the modifications disclosed herein, or combinations of said modifications. The actinoporin monomer may be a homologue monomer. A homologue monomer is a polypeptide that has at least about 40%, at least about 45%, at least about 50%, at least about 55%, at least about 60%, at least about 65%, at least about 70%, at least about 75%, at least about 80%, at least about 85%, at least about 90%, at least about 95% or at least about 99% complete sequence identity to SEQ ID NO: 1 over its entire length. A homologue monomer is typically an actinoporin monomer from a species different from Orbicella faveolata. The variant may comprise any of the substitutions present in a homologue monomer.

Sequence identity can also relate to a fragment or portion of the actinoporin monomer. Although a variant must have at least about 40% overall sequence identity with SEQ ID NO: 1 over its entire length, the sequence of a particular region, domain or subunit could share at least about 80%, 90%, or as much as about 99% sequence identity with the corresponding region of SEQ ID NO: 1. There may be at least about 80%, for example at least about 85%, 90% or 95%, identity over a stretch of 100 or more, for example 125, 150, 175 or 200 or more, consecutive or contiguous amino acids (“hard identity”). The actinoporin monomer preferably comprises a variant having at least about 40% identity to the cap region of SEQ ID NO: 1. More preferably, the variant has at least about 45%, at least about 50%, at least about 55%, at least about 60%, at least about 65%, at least about 70%, at least about 75%, at least about 80%, at least about 85%, at least about 90% and more preferably at least about 95%, 97% or 99% identity to the cap region of SEQ ID NO: 1. The cap region is residues V104 to M259 of SEQ ID NO: 1.

The actinoporin monomer preferably comprises a variant having at least about 40% identity to the constriction region of SEQ ID NO: 1. More preferably, the variant has at least about 45%, at least about 50%, at least about 55%, at least about 60%, at least about 65%, at least about 70%, at least about 75%, at least about 80%, at least about 85%, at least about 90% and more preferably at least about 95%, 97% or 99% identity to the constriction region of SEQ ID NO: 1. The constriction region is residues A80 to L89 of SEQ ID NO: 1.

The actinoporin monomer preferably comprises a variant having at least about 40% identity to the transmembrane alpha helical region of SEQ ID NO: 1. More preferably, the variant has at least about 45%, at least about 50%, at least about 55%, at least about 60%, at least about 65%, at least about 70%, at least about 75%, at least about 80%, at least about 85%, at least about 90% and more preferably at least about 95%, 97% or 99% identity to the transmembrane alpha helical region of SEQ ID NO: 1. The transmembrane alpha helical region is residues L66 to N103 or R77 to N103 of SEQ ID NO: 1.

The variant preferably comprises one or more modifications which improve the ability of an actinoporin pore formed from the monomer to interact with a target analyte. The one or more modifications are preferably one or more mutations. The one or more modifications or mutations are preferably one or more additions, one or more deletions, one or more substitutions, or any combination thereof. The one or more modifications or mutations are preferably one or more substitutions. Target analytes are discussed in more detail below. The target analyte is preferably an amino acid, a polypeptide, a protein, a nucleotide, a polynucleotide, a polynucleotide-polypeptide conjugate, a monosaccharide, an oligosaccharide, or a polysaccharide.

The actinoporin monomers preferably form actinoporin pores which have improved target analyte reading properties, i.e., display improved analyte capture and discrimination. In particular, actinoporin pores constructed from the actinoporin monomers preferably capture amino acids, polypeptides, proteins, nucleotides, and polynucleotides more easily than the wild type. In addition, actinoporin pores constructed from the actinoporin monomers preferably display an increased current range, which makes it easier to discriminate between different analytes, and a reduced variance of states, which increases the signal-to-noise ratio. In addition, the number of analytes or the number of polymer subunits in a polymer (e.g., the number of amino acids in a polypeptide or the number of nucleotides in a polynucleotide) contributing to the current as they move through actinoporin pores constructed from the monomers is decreased. This makes it easier to identify a direct relationship between the observed current as the analyte moves through the actinoporin pore and analyte sequence. The improved analyte reading properties of the actinoporin pores are achieved via five main mechanisms, namely by changes in the: sterics (increasing or decreasing the size of amino acid residues); charge (e.g., introducing positive charge to interact with the nucleic acid sequence); hydrogen bonding (e.g., introducing amino acids that can hydrogen bond to the base pairs); pi stacking (e.g., introducing amino acids that interact through delocalised electron pi systems); and/or alteration of the structure of the pore (e.g., introducing amino acids that increase the size of the vestibule and/or constriction).

Any one or more of these five mechanisms may be responsible for the improved properties of the actinoporin pores of the invention. For instance, an actinoporin pore of the invention may display improved analyte reading properties as a result of altered sterics, altered hydrogen bonding and an altered structure.

The introduction of bulky residues in the actinoporin monomer, such as phenylalanine (F), tryptophan (W), tyrosine (Y) or histidine (H), increases the sterics of the actinoporin pore. The introduction of aromatic residues in the actinoporin monomer, such as phenylalanine (F), tryptophan (W), tyrosine (Y) or histidine (H), also increases the pi staking in the actinoporin pore. The introduction of bulky or aromatic residues in the actinoporin monomer also alters the structure of the actinoporin pore, for instance by opening up the pore and increasing the size of the vestibule and/or constriction. This is described in more detail below.

The variant may comprise one or more modifications or mutations at one or more of the positions in any combination of the (a) cap region, (b) constriction region, and (c) transmembrane alpha helical region, namely in (a), (b), (c), (a) and (b), (b) and (c), (a) and (c), or (a), (b) and (c).

The cap region preferably comprises a modification or a mutation at one or more of the positions corresponding to N105, K107, A109, G111, D113, E115, G117-R119, Q121, E122, D133-N135, P137, S141-Y144, L147, G149, R151, N154, S171, I172, K174, S199, N201, D203, D215, S231, G232, Q244, T246, E248, H250, L252-C258 in SEQ ID NO: 1. The one or more modifications or mutations are preferably one or more substitutions of the amino acids at the positions with G, A, V, L, I, P, C, S, T, N, Q, F, Y, W, D, H, E, R or K.

The constriction region preferably comprises a modification or a mutation at one or more of the positions corresponding to the positions in SEQ ID NO: 1 shown in column 1. In each row, the amino acid at the position corresponding to the position in SEQ ID NO: 1 shown in column 1 is preferably be substituted with one of the amino acids in column 3. Column 2 indicates whether the position points inwards into the pore vestibule/channel (“In”) or out of the pore (“Out”).

| Position | In or out | Preferred substitutions |
|---|---|---|
| A80 | In | G, A, V, L, I, P, C, S, T, N, Q, F, Y, |
| | | W, D, H, E, R or K |
| G81 | In | G, A, V, L, I, P, C, S, T, N, Q, F, Y, |
| | | W, D, H, E, R or K |
| T82 | Out | G, A, V, L, I, P, C, F, Y or W |
| I83 | Out | G, A, V, L, I, P, C, F, Y or W |
| I84 | In | G, A, V, L, I, P, C, S, T, N, Q, F, Y, |
| | | W, D, H, E, R or K |
| A85 | In | G, A, V, L, I, P, C, S, T, N, Q, F, Y, |
| | | W, D, H, E, R or K |
| G86 | Out | G, A, V, L, I, P, C, F, Y or W |
| A87 | Out | G, A, V, L, I, P, C, F, Y or W |
| E88 | In | G, A, V, L, I, P, C, S, T, N, Q, F, Y, |
| | | W, D, H, E, R or K |
| L89 | In | G, A, V, L, I, P, C, S, T, N, Q, F, Y, |
| | | W, D, H, E, R or K |

The transmembrane region preferably comprises a modification or a mutation at one or more of the positions corresponding to the positions in SEQ ID NO: 1 shown in column 1. In each row, the amino acid at the position corresponding to the position in SEQ ID NO: 1 shown in column 1 is preferably be substituted with one of the amino acids in column 3. Column 2 indicates whether the position points inwards into the pore vestibule/channel (“In”) or out of the pore (“Out”).

| Position | In or out | Preferred substitutions |
|---|---|---|
| I78 | Out | G, A, V, L, I, P, C, F, Y or W |
| A79 | Out | G, A, V, L, I, P, C, F, Y or W |
| T90 | Out | G, A, V, L, I, P, C, F, Y or W |
| I91 | Out | G, A, V, L, I, P, C, F, Y or W |
| G92 | In | G, A, V, L, I, P, C, S, T, N, Q, F, Y, |
| | | W, D, H, E, R or K |
| L93 | Out | G, A, V, L, I, P, C, F, Y or W |
| L94 | Out | G, A, V, L, I, P, C, F, Y or W |
| Q95 | In | G, A, V, L, I, P, C, S, T, N, Q, F, Y, |
| | | W, D, H, E, R or K |
| N96 | In | G, A, V, L, I, P, C, S, T, N, Q, F, Y, |
| | | W, D, H, E, R or K |
| L97 | Out | G, A, V, L, I, P, C, F, Y or W |
| L98 | Out | G, A, V, L, I, P, C, F, Y or W |
| D99 | In | G, A, V, L, I, P, C, S, T, N, Q, F, Y, |
| | | W, D, H, E, R or K |
| V100 | In | G, A, V, L, I, P, C, S, T, N, Q, F, Y, |
| | | W, D, H, E, R or K |
| L101 | Out | G, A, V, L, I, P, C, F, Y or W |
| A102 | Out | G, A, V, L, I, P, C, F, Y or W |
| N103 | In | G, A, V, L, I, P, C, S, T, N, Q, F, Y, |
| | | W, D, H, E, R or K |

The variant may comprise any number of one or more modifications or mutations, such as 1, 2, 3, 4, 5, 6, 7, 8, 9, 10, 11, 12, 13, 14, 15, 16, 17, 18, 19, 20 or more modifications or mutations. The one or more modifications or mutations are preferably one or more additions, one or more deletions, one or more substitutions, or any combination thereof. The one or more modifications or mutations are preferably one or more substitutions.

The one or more modifications or mutations each independently (a) alter the size of the amino acid residue at the modified position; (b) alter the net charge of the amino acid residue at the modified position; (c) alter the hydrogen bonding characteristics of the amino acid residue at the modified position; (d) introduce to or remove from the amino acid residue at the modified position one or more chemical groups that interact through delocalized electron pi systems and/or (e) alter the structure of the amino acid residue at the modified position. The one or more modifications or mutations are preferably one or more additions, one or more deletions, one or more substitutions, or any combination thereof. The one or more modification or mutations are preferably one or more substitutions.

The variant preferably comprises one or more modifications or mutations which improve the ability of an actinoporin pore formed from the monomer to interact with an amino acid, a polypeptide, or a protein. The terms “polypeptides” and “proteins” are interchangeable and are discussed in more detail. The one or more modifications are preferably one or more additions, one or more deletions, one or more substitutions, or any combination thereof. The one or more modifications are preferably one or more substitutions.

Depending on the charge of the amino acid or polypeptide analyte, the variant preferably comprises one or more modifications or mutations which reduce the same net charge of the monomer. If the amino acid or polypeptide analyte is positively charged, the variant preferably comprises one or more modifications or mutations which reduce the net positive charge of the monomer. If the amino acid or polypeptide analyte is negatively charged, the variant preferably comprises one or more modifications or mutations which reduce the net negative charge of the monomer. This reduces repulsion and promotes capture of the amino acid or polypeptide analyte.

If the amino acid or polypeptide analyte is positively charged, one or more modifications or mutations are preferably made to include negative, polar, or hydrophobic amino acids in order to either reduce positivity (so that the capture of positively charged analytes is promoted). For instance, positively charged amino acids, such as R, H or K, may be substituted with negative, polar, or hydrophobic amino acids. Amino acids may be substituted with D, E, S, T, N, Q, G, A, V, L, I, P, C, F, Y or W. One or more modifications or mutations are preferably made to include polar, hydrophobic, positive, or negative residues in order to change the discrimination. Amino acids may be substituted with S, T, N, Q, G, A, V, L, I, P, C, R, K, D, E, F, Y, or W.

If the amino acid or polypeptide analyte is positively charged, the one or more modifications or mutations are preferably selected from the following specific substitutions:
R to D, E, S, T, N, Q, G, A, V, L, I, C, F, Y, or W,
K to D, E, S, T, N, Q, G, A, V, L, I, C, F, Y, or W,
D to S, T, N, Q, G, A, V, L, I, C, F, Y, or W, preferably D to N, Q, or A,
E to S, T, N, Q, G, A, V, L, I, C, F, Y, or W, preferably to E to N, Q, or A,
S to E, D, T, N, Q, A, V, L, or I, preferably S to E or D,
T to E, D, S, N, Q, A, V, L, or I, preferably T to E or D,
N to E, D, S, T, Q, A, V, L, or I preferably N to E or D, and
Q to E, D, S, T, N, A, V, L, or I, preferably Q to E or D.

If the amino acid or polypeptide analyte is positively charged, the variant preferably comprises a modification or mutation at one or more of the positions corresponding to E67, N68, K73, R74, R77, I91, D99, V100, L101, D215, L252 and E254 in SEQ ID NO: 1. The variant may comprise any number of modifications or mutations at these positions, such as 1, 2, 3, 4, 5, 6, 7, 8, 9, 10, 11 or 12 of these positions. The variant preferably comprises a modification or mutation at all of positions corresponding to E67, N68, K73, R74, R77, I91, D99, V100, L101, D215, L252 and E254 in SEQ ID NO: 1. The one or more modifications or mutations are preferably one or more substitutions. The amino acids at these positions may be modified, mutated, or substituted in any of the ways discussed above. The variant preferably comprises a modification or mutation at the position corresponding to D215 in SEQ ID NO: 1. The variant preferably comprises a modification or mutation at one or more of, such as all of, the positions corresponding to E67, N68, K73, R74, R77, I91, D99, V100, L101 and D215 in SEQ ID NO: 1. The variant preferably comprises a modification or mutation at one or more of, such as all of, the positions corresponding to D215, L252 and E254 in SEQ ID NO: 1. Any of these variants preferably further comprises a mutation or modification, preferably a substitution, at position D203. This is discussed in more detail below.

If the amino acid or polypeptide analyte is positively charged, the variant preferably comprises:
one or more of or all of E67S/N68E/K73N/R74D/R77D/I91F/D99Y/V100F/L101F/D215R/L252T/E254A,
one or more of or all of E67S/N68E/K73N/R74D/R77D/I91F/D99Y/V100F/L101F/D203R/D215R/L252T/E254A,
D203R,
D215R,
D203R/D215R,
one or more of or all of E67S/N68E/K73N/R74D/R77D/I91F/D99Y/V100F/L101F/D215R,
one or more of or all of E67S/N68E/K73N/R74D/R77D/I91F/D99Y/V100F/L101F/D203R/D215R,
one or more of or all of D215R/L252T/E254A,
one or more of or all of D203R/D215R/L252T/E254A
one or more of or all of E67S/N68E/K73N/R74D/R77D/I91F/D99Y/V100F/L101F/D215N/L252T/E254A,
one or more of or all of E67S/N68E/K73N/R74D/R77D/I91F/D99Y/V100F/L101F/D203R/D215N/L252T/E254A
D215N,
D203R/D215N,
one or more of or all of E67S/N68E/K73N/R74D/R77D/I91F/D99Y/V100F/L101F/D215N,
one or more of or all of E67S/N68E/K73N/R74D/R77D/I91F/D99Y/V100F/L101F/D203R/D215N,
one or more of or all of D215N/L252T/E254A or
one or more of or all of D203R/D215N/L252T/E254A.

Examples of positively charged polypeptides and proteins are provided below.

If the amino acid or polypeptide analyte is negatively charged, one or more modifications or mutations are preferably made to include positive, polar, or hydrophobic amino acids in order to either reduce negativity (so that the capture of negatively charged analyte is promoted) or improve/stabilise the interaction with the polynucleotide binding protein (discussed in more detail below). For instance, negatively charged amino acids, such as D or E, may be substituted with positive, polar, or hydrophobic amino acids. Amino acids may be substituted with R, K, S, T, N, Q, G, A, V, L, I, P, C, F, Y or W. One or more modifications or mutations are preferably made to include polar, hydrophobic, positive, or negative residues in order to change the discrimination. Amino acids may be substituted with S, T, N, Q, G, A, V, L, I, P, C, R, K, D, E, F, Y, or W.

The variant preferably comprises one or more modifications or mutations which improve the ability of an actinoporin pore formed from the monomer to interact with a nucleotide or a polynucleotide. Nucleotides and polynucleotides are discussed in more detail below. The one or more modifications or mutations are preferably one or more additions, one or more deletions, one or more substitutions, or any combination thereof. The one or more modifications or mutations are preferably one or more substitutions.

Nucleotides and polynucleotides are typically negatively charged. One or more modifications or mutations are preferably made to include positive, polar, or hydrophobic amino acids in order to either reduce negativity (so that the capture of negatively charged analytes is promoted) or improve/stabilise the interaction with the polynucleotide binding protein. Amino acids may be substituted with R, K, S, T, N, Q, G, A, V, L, I, P, C, F, Y or W. One or more modifications or mutations are preferably made to include polar, hydrophobic, positive, or negative in order to change the discrimination. Amino acids may be substituted with S, T, N, Q, G, A, V, L, I, P, C, R, K, D, E, F, Y, or W.

For target nucleotides or polynucleotides, the one or more mutations are preferably selected from the following specific substitutions:
D to S, T, N, Q, A, V, L, I, F, R, or K,
E to S, T, N, Q, A, V, L, I, F, R, or K,
R to S, T, N, Q, A, V, L, I, or F,
K to S, T, N, Q, A, V, L, I, or F,
G to S, T, N, Q or A, V, L, I, or F,
A to S, T, N, Q or G, V, L, I, or F,
V to S, T, N, Q or G, A, L, I, or F,
L to S, T, N, Q or G, A, V, I, or F,
I to S, T, N, Q or G, A, V, L, or F,
S to T, N, Q, A, V, L, I, F, R, or K,
T to S, N, Q, A, V, L, I, F, R, or K,
N to S, T, Q, A, V, L, I, F, R, or K,
Q to S, T, N, A, V, L, I, F, R, or K, and
H to F, N, Q, or A.

For target nucleotides or polynucleotides, the variant preferably comprises a modification or mutation at one or more of the positions corresponding to positions D8, E12, E15, E16, E25, D30, E38, E45, E57, E62, D65, E67, D70, E72, E88, D99, D113, E115, E122, D133, E134, D142, D185, D203, D205, D207, D211, D215, E226, E248, E254 and E256 in SEQ ID NO: 1. The variant may comprise any number of modifications or mutations at these positions, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32 of these positions. The variant preferably comprises a modification or mutation at one or more of, such as all of, the positions corresponding to positions E67, D70, E72, E88, D113, E115, E134, D142, E248 and E256 in SEQ ID NO: 1. The variant may comprise any number of modifications or mutations at these positions, such as 1, 2, 3, 4, 5, 6, 7, 8, 9, or 10 of these positions. The variant preferably comprises a modification or mutation at one or more of, such as all of, the positions corresponding to positions E67, D70 and E72 in SEQ ID NO: 1. These positions are in the N-terminus. The variant may comprise any number of modifications or mutations at these positions, such as 1, 2 or 3 of these positions. The variant preferably comprises a modification or mutation at one or more of, such as all of, the positions corresponding to positions D113, E115, E134, D142, E248 and E256 in SEQ ID NO: 1. These positions are in the pore entrance. The variant may comprise any number of modifications or mutations at these positions, such as 1, 2, 3, 4, 5 or 6 of these positions. The variant preferably comprises a modification or mutation at the position corresponding to position E88 in SEQ ID NO: 1. This position is in the transmembrane alpha helical region. The one or more modifications are preferably one or more additions, one or more deletions, one or more substitutions, or any combination thereof. The one or more modifications are preferably one or more substitutions.

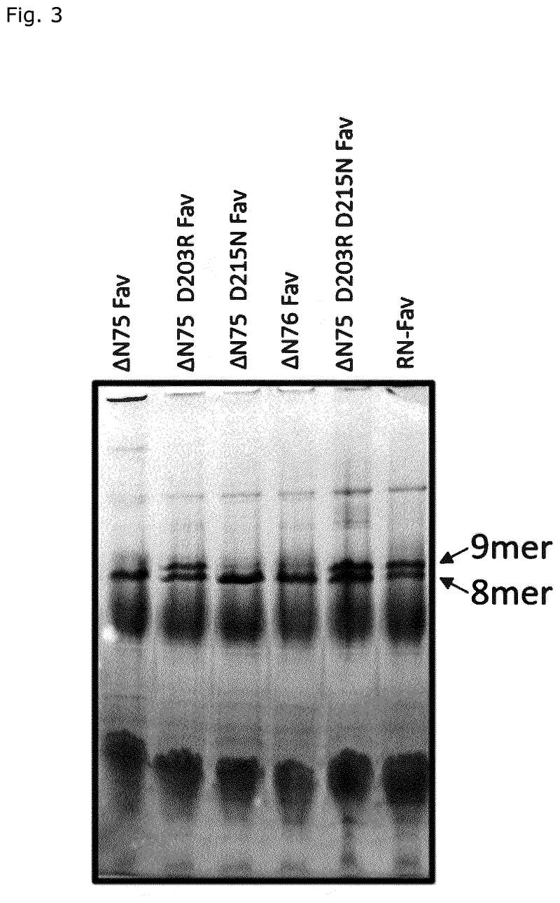

The variant preferably comprises one or more modifications or mutations which alter the number of monomers which form an actinoporin pore. The one or more modifications or mutations are preferably one or more additions, one or more deletions, one or more substitutions, or any combination thereof. The variant preferably comprises one or more substitutions which alter the number of monomers which form an actinoporin pore comprising the monomer. The one or more modifications, mutations or substitutions preferably increase the number of monomers forming an actinoporin pore. The one or more modifications, mutations or substitutions preferably increase the number of monomers forming an actinoporin pore. The one or more modifications, mutations or substitutions preferably increase the number of monomers forming an actinoporin pore from 8 to 9. The variant preferably comprises a modification, such as an addition, deletion or substitution, or a substitution, at the position corresponding to D203 in SEQ ID NO: 1. The variant preferably comprises D203R/N/K/R/S/Y/F/A or D203R/K at the corresponding position. The variant most preferably comprises D203R at the corresponding position. This substitution increases the number of monomers forming an actinoporin pore from mostly 8 to 8/9 in a 1:1 ratio (FIG. 3). These modifications, mutations or substitutions may be in addition to or instead of any of the modifications or mutations discussed above for improving the ability of an actinoporin pore to interact with a target analyte.

The variant is preferably a fragment lacking one or more amino acids from the N-terminus of SEQ ID NO: 1. This facilitates the insertion of the monomer into an artificial membrane. The fragment may lack any number of amino acids from the N-terminus of SEQ ID NO: 1 as long as the variant has at least about 40% identity to the sequence of SEQ ID NO: 1 over its entire length. The fragment preferably lacks up to about 108 amino acids from the N-terminus of SEQ ID NO: 1. The fragment may lack up to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107 or 108 amino acids from the N-terminus of SEQ ID NO: 1. The fragment preferably lacks at least about 53 amino acids from the N-terminus of SEQ ID NO: 1. The fragment preferably lacks about 53 amino acids from the N-terminus of SEQ ID NO: 1. The fragment preferably lacks at least about 67 amino acids from the N-terminus of SEQ ID NO: 1. The fragment preferably lacks about 67 amino acids from the N-terminus of SEQ ID NO: 1. The fragment preferably lacks at least about 76 amino acids from the N-terminus of SEQ ID NO: 1. The fragment preferably lacks about 76 amino acids from the N-terminus of SEQ ID NO: 1. The fragment may include any of the modifications or mutations discussed above for improving the ability of an actinoporin pore to interact with a target analyte and/or altering the number of monomers which form a actinoporin pore.

The actinoporin monomers of the invention are preferably capable of inserting into an artificial membrane. The variant preferably comprises one or more modifications which facilitate insertion of the actinoporin monomer into an artificial membrane. Insertion can be measured using routine methods, including any of those described in WO 2016/034591, WO 2017/149316, WO 2017/149317, WO 2017/149318, WO 2018/211241, and WO 2019/002893 (all incorporated by reference herein in their entirety) and in the Examples.

Different N-terminal deletions from SEQ ID NO: 1 are capable of altering the ratio of different sized actinoporin pore oligomers, especially when combined with the modifications, mutations, or substitutions for altering the number of monomers discussed above. Variants of SEQ ID NO: 1 having D203R at the corresponding position and lacking 67 amino acids from the N-terminus of SEQ ID NO: 1 are capable of forming 8-mer and 9-mer actinoporin pore oligomers in a ratio of about 1:3. Variants of SEQ ID NO: 1 having D203R at the corresponding position and lacking 76 amino acids from the N-terminus of SEQ ID NO: 1 are capable of forming 8-mer and 9-mer actinoporin pore oligomers in a ratio of about 1:1.

The variant is preferably a fragment lacking one or more amino acids from the C-terminus of SEQ ID NO: 1. The fragment may lack any number of amino acids from the C-terminus of SEQ ID NO: 1 as long as the variant has at least about 40% identity to the sequence of SEQ ID NO: 1 over its entire length. The fragment preferably lacks up to about 10 amino acids from the C-terminus of SEQ ID NO: 1. The fragment may lack up to about 1, 2, 3, 4, 5, 6, 7, 8, 9, or 10 amino acids from the C-terminus of SEQ ID NO: 1. The fragment preferably lacks up to or at least about 5 amino acids from the C-terminus of SEQ ID NO: 1. The fragment preferably lacks about 5 amino acids from the C-terminus of SEQ ID NO: 1. The fragment may include any of the modifications or mutations discussed above for improving the ability of an actinoporin pore to interact with a target analyte and/or altering the number of monomers which form an actinoporin pore. Fragments in accordance with invention may lack one or more amino acids from the N-terminus of SEQ ID NO: 1 and one or more amino acids from the C-terminus of SEQ ID NO: 1.

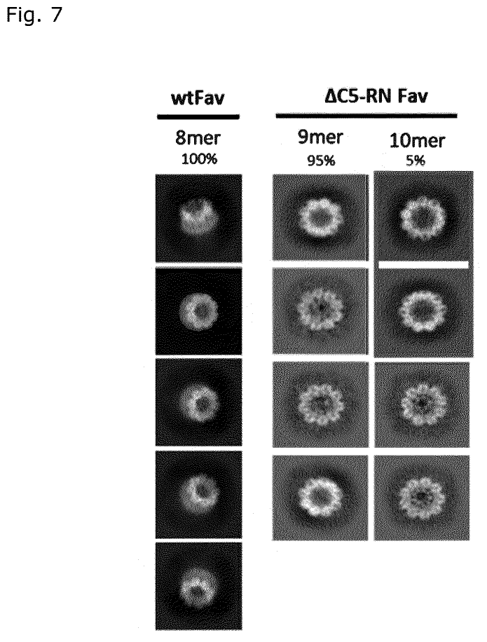

Deletion of one or more amino acids from the C-terminus of SEQ ID NO: 1, such as about 5 amino acids from the C-terminus of SEQ ID NO: 1, is also capable in combination with a modification or mutation at the position corresponding to D203 in SEQ ID NO: 1 of increasing the number of monomers which form an actinoporin pore. Variants including a mutation or modification, such as a substitution, at the position corresponding to position D203 in SEQ ID NO: 1, such as D203R, and lacking 5 amino acids from the C-terminus of SEQ ID NO: 1 are capable of forming 9-mer and 10-mer actinoporin pore oligomers (FIG. 7).

The actinoporin monomer typically retains the ability to form the same 3D structure as the wild type actinoporin monomer, such as the same 3D structure as an actinoporin monomer having the sequence of SEQ ID NO: 1. Any number of modifications or mutations may be made in addition to the modifications or mutations described herein provided that the actinoporin monomer retains the improved properties imparted on it by the modifications or mutations of the present invention.

Typically, the actinoporin monomer will retain the ability to form a structure comprising an alpha helix or a transmembrane alpha helix. Therefore, it is envisaged that further mutations may be made without affecting the ability of the monomer to form an actinoporin pore that can translocate analytes. It is also expected that deletions of one or more amino acids can be made in any of the loop regions and/or in the N-terminal and/or C-terminal regions of the actinoporin monomer without affecting the ability of the monomer to form a pore that can translocate analytes.

Amino acid substitutions may be made to the amino acid sequence of SEQ ID NO: 1 in addition to those discussed above, for example up to 1, 2, 3, 4, 5, 10, 20 or 30 substitutions. Conservative substitutions replace amino acids with other amino acids of similar chemical structure, similar chemical properties, or similar side-chain volume. The amino acids introduced may have similar polarity, hydrophilicity, hydrophobicity, basicity, acidity, neutrality, or charge to the amino acids they replace. Alternatively, the conservative substitution may introduce another amino acid that is aromatic or aliphatic in the place of a pre-existing aromatic or aliphatic amino acid. Conservative amino acid changes are well-known in the art.

The actinoporin monomer may be modified to introduce one or more cysteines, one or more hydrophobic amino acids, one or more charged amino acids, one or more non-native amino acids, one or more polar amino acids, or one or more photoreactive amino acids. Any number and combination of such introductions may be made. The introduction is preferably by substitution or addition.

One or more amino acid residues of the amino acid sequence of SEQ ID NO: 1 may additionally be deleted from the polypeptides described above. Up to 1, 2, 3, 4, 5, 10, 20 or 30 or more residues may be deleted.

One or more amino acids may be alternatively or additionally added to the polypeptides described above. An extension may be provided at the amino terminal or carboxy terminal of the amino acid sequence of SEQ ID NO: 1 or variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

The one or more modifications in the actinoporin monomer preferably improve the ability of an actinoporin pore comprising the actinoporin monomer to characterise an analyte. For example, modifications, or substitutions are contemplated to alter the number, size, shape, placement, or orientation of the constriction within a channel from the actinoporin monomer of the invention. The actinoporin monomer or the variant of SEQ ID NO: 1 may have any of the particular modifications or substitutions disclosed in WO 2016/034591, WO 2017/149316, WO 2017/149317, WO 2017/149318, WO 2018/211241, and WO 2019/002893 (all incorporated by reference herein in their entirety).

Any actinoporin monomer and/or variant based on SEQ ID NO: 1 described herein preferably further comprises the sequence GHM or the amino acid S at its N-terminus.

### Actinoporin Monomers Based on SEQ ID NO: 2

The invention provides an actinoporin monomer comprising a variant of SEQ ID NO: 2 having at least about 54% identity to the sequence of SEQ ID NO: 2 over its entire length. The variant preferably has at least about 55%, at least about 60%, at least about 65%, at least about 70%, at least about 75%, at least about 80%, at least about 85%, or at least about 90% identity to the sequence of SEQ ID NO: 2 over its entire length. The variant more preferably has at least about 95%, at least about 97%, at least about 98% or at least about 99% identity to the sequence of SEQ ID NO: 2 over its entire length.

Standard methods in the art may be used to determine identity. For example, the UWGCG Package provides the BESTFIT program which can be used to calculate identity, for example used on its default settings (Devereux et al (1984) Nucleic Acids Research 12, p 387-395). The PILEUP and BLAST algorithms can be used to calculate identity or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

The % identity of the variant to SEQ ID NO: 2 is measured over the entire length of SEQ ID NO: 2. SEQ ID NO: 2 is 183 amino acids long. Identity is typically determined by calculating the number of identical residues between the variant and SEQ ID NO: 2 using any of the methods above and dividing the number of identical residues by 183. For instance, if a variant has 105 identical amino acids to SEQ ID NO: 2, the variant has 105/183×100=57.38% identity to SEQ ID NO: 2 over its entire length.

The variant preferably comprises one or more modifications which (a) improve the ability of an actinoporin pore formed from the monomer to interact with a target analyte, (b) alter the number of actinoporin monomers which form an actinoporin pore and/or (c) facilitate insertion of the actinoporin monomer or an actinoporin pore formed from the monomer into an artificial membrane. The variant may comprise (a), (b), (c), (a) and (b), (a) and (c), (b) and (c), or (a), (b) and (c). Each of (a)-(c) are described in more detail below. The one or more modifications are preferably one or more mutations. The one or more modifications or mutations are preferably one or more additions, one or more deletions, one or more substitutions, or any combination thereof. The one or more modifications or mutations are preferably one or more substitutions.

The actinoporin monomers of the invention are typically capable of forming a pore or an actinoporin pore. This can be measured using routine methods, including any of those described in WO 2016/034591, WO 2017/149316, WO 2017/149317, WO 2017/149318, WO 2018/211241, and WO 2019/002893 (all incorporated by reference herein in their entirety) and in the Examples.

The actinoporin monomer preferably comprises one or more of (a) a cap region, (b) a constriction region, and (c) a transmembrane alpha helical region, such as (a), (b), (c), (a) and (b), (a) and (c), (b) and (c), or (a), (b) and (c). The actinoporin monomer preferably includes one or more regions which contribute to these regions in the actinoporin pore. The actinoporin monomer more preferably comprises one or more of regions which form one or more of (a)-(c), such as (a), (b), (c), (a) and (b), (a) and (c), (b) and (c), or (a), (b) and (c). The actinoporin pore formed by the monomer may have any structure but preferably has or comprises the structure of the wild type actinoporin pore. The protein structure of actinoporin pore defines a channel or hole that allows the translocation of molecules and ions from one side of the membrane to the other.

The constriction”, “orifice”, “constriction region”, “channel constriction”, or “constriction site”, as used interchangeably herein, refers to an aperture defined by a luminal surface of a pore or actinoporin pore, which acts to allow the passage of ions and target analytes (including but not limited to polynucleotides or individual nucleotides) but not other non-target analytes through the pore or actinoporin pore channel. In some instances, for example when characterising protein or polypeptide target analytes, the constriction prevents the target analyte from passing through the pore. The constriction(s) are typically the narrowest aperture(s) within a pore or actinoporin pore or within the channel defined by the pore or actinoporin pore. The constriction(s) may serve to limit the passage of molecules through the pore. The size of the constriction is typically a key factor in determining suitability of a pore or actinoporin pore for analyte characterisation. If the constriction is too small, the molecule to be characterised will not be able to pass through. However, to achieve a maximal effect on ion flow through the channel, the constriction should not be too large. For example, the constriction should not be wider than the solvent-accessible transverse diameter of a target analyte. Ideally, any constriction should be as close as possible in diameter to the transverse diameter of the analyte passing through.

SEQ ID NO: 2 shows the sequence of the wild type actinoporin monomer derived from Orbicella faveolata as a mature protein (SEQ ID NO: 1) and lacking the first 76 amino acids from the N-terminus. Residues V28 to M183 of SEQ ID NO: 2 form the cap region. Residues A4 to L13 of SEQ ID NO: 2 form the constriction region. Residues R1 to N27 of SEQ ID NO: 2 form the transmembrane alpha helical region. The constriction region is typically present in the transmembrane alpha helical region.

The actinoporin monomer comprises a variant of SEQ ID NO: 2. The variant monomer may also be referred to as a modified actinoporin monomer or a mutant actinoporin monomer. The modifications, or mutations, in the variant include but are not limited to any one or more of the modifications disclosed herein, or combinations of said modifications. The actinoporin monomer may be a homologue monomer. A homologue monomer is a polypeptide that has at least about 55%, at least about 60%, at least about 65%, at least about 70%, at least about 75%, at least about 80%, at least about 85%, at least about 90%, at least about 95% or at least about 99% complete sequence identity to SEQ ID NO: 2 over its entire length. A homologue monomer is typically an actinoporin monomer from a species different from Orbicella faveolata. The variant may comprise any of the substitutions present in a homologue monomer.

Sequence identity can also relate to a fragment or portion of the actinoporin monomer. Although a variant must have at least about 54% overall sequence identity with SEQ ID NO: 2 over its entire length, the sequence of a particular region, domain or subunit could share at least about 80%, 90%, or as much as about 99% sequence identity with the corresponding region of SEQ ID NO: 2. There may be at least about 80%, for example at least about 85%, 90% or 95%, identity over a stretch of 100 or more, for example 125, 150, 175 or 200 or more, consecutive or contiguous amino acids (“hard identity”). The actinoporin monomer preferably comprises a variant having at least about 40% identity to the cap region of SEQ ID NO: 2. More preferably, the variant has at least about 45%, at least about 50%, at least about 55%, at least about 60%, at least about 65%, at least about 70%, at least about 75%, at least about 80%, at least about 85%, at least about 90% and more preferably at least about 95%, 97% or 99% identity to the cap region of SEQ ID NO: 2. The cap region is residues V28 to M183 of SEQ ID NO: 2.

The actinoporin monomer preferably comprises a variant having at least about 54% identity to the constriction region of SEQ ID NO: 2. More preferably, the variant has at least about 55%, at least about 60%, at least about 65%, at least about 70%, at least about 75%, at least about 80%, at least about 85%, at least about 90% and more preferably at least about 95%, 97% or 99% identity to the constriction region of SEQ ID NO: 2. The constriction region is residues A4 to L13 of SEQ ID NO: 2.

The actinoporin monomer preferably comprises a variant having at least about 54% identity to the transmembrane alpha helical region of SEQ ID NO: 2. More preferably, the variant has at least about 55%, at least about 60%, at least about 65%, at least about 70%, at least about 75%, at least about 80%, at least about 85%, at least about 90% and more preferably at least about 95%, 97% or 99% identity to the transmembrane alpha helical region of SEQ ID NO: 2. The transmembrane alpha helical region is residues R1 to N27 of SEQ ID NO: 2.

The variant preferably comprises one or more modifications which improve the ability of an actinoporin pore formed from the monomer to interact with a target analyte. The one or more modifications are preferably one or more mutations. The one or more modifications or mutations are preferably one or more additions, one or more deletions, one or more substitutions, or any combination thereof. The one or more modifications or mutations are preferably one or more substitutions. Target analytes are discussed in more detail below. The target analyte is preferably an amino acid, a polypeptide, a protein, a nucleotide, a polynucleotide a polynucleotide-polypeptide conjugate, a monosaccharide, an oligosaccharide, a polysaccharide.

The actinoporin monomers preferably form actinoporin pores which have improved target analyte reading properties, i.e., display improved analyte capture and discrimination. In particular, actinoporin pores constructed from the actinoporin monomers preferably capture amino acids, polypeptides, proteins, nucleotides, and polynucleotides more easily than the wild type. In addition, actinoporin pores constructed from the actinoporin monomers preferably display an increased current range, which makes it easier to discriminate between different analytes, and a reduced variance of states, which increases the signal-to-noise ratio. In addition, the number of analytes or the number of polymer subunits in a polymer (e.g., the number of amino acids in a polypeptide or the number of nucleotides in a polynucleotide) contributing to the current as they move through actinoporin pores constructed from the monomers is decreased. This makes it easier to identify a direct relationship between the observed current as the analyte moves through the actinoporin pore and analyte sequence. The improved analyte reading properties of the actinoporin pores are achieved via five main mechanisms, namely by changes in the: sterics (increasing or decreasing the size of amino acid residues); charge (e.g., introducing positive charge to interact with the nucleic acid sequence); hydrogen bonding (e.g., introducing amino acids that can hydrogen bond to the base pairs); pi stacking (e.g., introducing amino acids that interact through delocalised electron pi systems); and/or alteration of the structure of the pore (e.g., introducing amino acids that increase the size of the vestibule and/or constriction).

Any one or more of these five mechanisms may be responsible for the improved properties of the actinoporin pores of the invention. For instance, an actinoporin pore of the invention may display improved analyte reading properties as a result of altered sterics, altered hydrogen bonding and an altered structure.

The introduction of bulky residues in the actinoporin monomer, such as phenylalanine (F), tryptophan (W), tyrosine (Y) or histidine (H), increases the sterics of the actinoporin pore. The introduction of aromatic residues in the actinoporin monomer, such as phenylalanine (F), tryptophan (W), tyrosine (Y) or histidine (H), also increases the pi staking in the actinoporin pore. The introduction of bulky or aromatic residues in the actinoporin monomer also alters the structure of the actinoporin pore, for instance by opening up the pore and increasing the size of the vestibule and/or constriction. This is described in more detail below.

The variant may comprise one or more modifications or mutations at one or more of the positions in any combination of the (a) cap region, (b) constriction region, and (c) transmembrane alpha helical region, namely in (a), (b), (c), (a) and (b), (b) and (c), (a) and (c), or (a), (b) and (c).

The cap region preferably comprises a modification or a mutation at one or more of the positions corresponding to N29, K31, A33, G35, D37, E39, G41-R43, Q45, E46, D57-N59, P61, S65-Y68, L71, G73, R75, N78, S95, I96, K98, S123, N125, D127, D139, S155, G156, Q168, T170, E172, H174, L176-C182 in SEQ ID NO: 2. The one or more modifications or mutations are preferably one or more substitutions of the amino acids at the positions with G, A, V, L, I, P, C, S, T, N, Q, F, Y, W, D, H, E, R or K.

The constriction region preferably comprises a modification or a mutation at one or more of the positions corresponding to the positions in SEQ ID NO: 2 shown in column 1. In each row, the amino acid at the position corresponding to the position in SEQ ID NO: 2 shown in column 1 is preferably be substituted with one of the amino acids in column 3. Column 2 indicates whether the position points inwards into the pore vestibule/channel (“In”) or out of the pore (“Out”).

| Position | In or out | Preferred substitutions |
|---|---|---|
| A4 | In | G, A, V, L, I, P, C, S, T, N, Q, F, Y, |
| | | W, D, H, E, R or K |
| G5 | In | G, A, V, L, I, P, C, S, T, N, Q, F, Y, |
| | | W, D, H, E, R or K |
| T6 | Out | G, A, V, L, I, P, C, F, Y or W |
| I7 | Out | G, A, V, L, I, P, C, F, Y or W |
| I8 | In | G, A, V, L, I, P, C, S, T, N, Q, F, Y, |
| | | W, D, H, E, R or K |
| A9 | In | G, A, V, L, I, P, C, S, T, N, Q, F, Y, |
| | | W, D, H, E, R or K |
| G10 | Out | G, A, V, L, I, P, C, F, Y or W |
| A11 | Out | G, A, V, L, I, P, C, F, Y or W |
| E12 | In | G, A, V, L, I, P, C, S, T, N, Q, F, Y, |
| | | W, D, H, E, R or K |
| L13 | In | G, A, V, L, I, P, C, S, T, N, Q, F, Y, |
| | | W, D, H, E, R or K |

The transmembrane region preferably comprises a modification or a mutation at one or more of the positions corresponding to the positions in SEQ ID NO: 2 shown in column 1. In each row, the amino acid at the position corresponding to the position in SEQ ID NO: 2 shown in column 1 is preferably be substituted with one of the amino acids in column 3. Column 2 indicates whether the position points inwards into the pore vestibule/channel (“In”) or out of the pore (“Out”).

| Position | In or out | Preferred substitutions |
|---|---|---|
| I2 | Out | G, A, V, L, I, P, C, F, Y or W |
| A3 | Out | G, A, V, L, I, P, C, F, Y or W |
| T14 | Out | G, A, V, L, I, P, C, F, Y or W |
| I15 | Out | G, A, V, L, I, P, C, F, Y or W |
| G16 | In | G, A, V, L, I, P, C, S, T, N, Q, F, Y, |
| | | W, D, H, E, R or K |
| L17 | Out | G, A, V, L, I, P, C, F, Y or W |
| L18 | Out | G, A, V, L, I, P, C, F, Y or W |
| Q19 | In | G, A, V, L, I, P, C, S, T, N, Q, F, Y, |
| | | W, D, H, E, R or K |
| N20 | In | G, A, V, L, I, P, C, S, T, N, Q, F, Y, |
| | | W, D, H, E, R or K |
| L21 | Out | G, A, V, L, I, P, C, F, Y or W |
| L22 | Out | G, A, V, L, I, P, C, F, Y or W |
| D23 | In | G, A, V, L, I, P, C, S, T, N, Q, F, Y, |
| | | W, D, H, E, R or K |
| V24 | In | G, A, V, L, I, P, C, S, T, N, Q, F, Y, |
| | | W, D, H, E, R or K |
| L25 | Out | G, A, V, L, I, P, C, F, Y or W |
| A26 | Out | G, A, V, L, I, P, C, F, Y or W |
| N27 | In | G, A, V, L, I, P, C, S, T, N, Q, F, Y, |
| | | W, D, H, E, R or K |

The variant may comprise any number of one or more modifications or mutations, such as 1, 2, 3, 4, 5, 6, 7, 8, 9, 10, 11, 12, 13, 14, 15, 16, 17, 18, 19, 20 or more modifications or mutations. The one or more modifications or mutations are preferably one or more additions, one or more deletions, one or more substitutions, or any combination thereof. The one or more modifications or mutations are preferably one or more substitutions.

The one or more modifications or mutations each independently (a) alter the size of the amino acid residue at the modified position; (b) alter the net charge of the amino acid residue at the modified position; (c) alter the hydrogen bonding characteristics of the amino acid residue at the modified position; (d) introduce to or remove from the amino acid residue at the modified position one or more chemical groups that interact through delocalized electron pi systems and/or (e) alter the structure of the amino acid residue at the modified position. The one or more modifications or mutations are preferably one or more additions, one or more deletions, one or more substitutions, or any combination thereof. The one or more modification or mutations are preferably one or more substitutions.

The variant preferably comprises one or more modifications or mutations which improve the ability of an actinoporin pore formed from the monomer to interact with an amino acid, a polypeptide, or a protein. The terms “polypeptides” and “proteins” are interchangeable and are discussed in more detail. The one or more modifications or mutations are preferably one or more additions, one or more deletions, one or more substitutions, or any combination thereof. The one or more modifications are preferably one or more substitutions.

Depending on the charge of the amino acid or polypeptide analyte, the variant preferably comprises one or more modifications or mutations which reduce the same net charge of the monomer. If the amino acid or polypeptide analyte is positively charged, the variant preferably comprises one or more modifications or mutations which reduce the net positive charge of the monomer. If the amino acid or polypeptide analyte is negatively charged, the variant preferably comprises one or more modifications or mutations which reduce the net negative charge of the monomer. This reduces repulsion and promotes capture of the amino acid or polypeptide analyte.

If the amino acid or polypeptide analyte is positively charged, one or more modifications or mutations are preferably made to include negative, polar, or hydrophobic amino acids in order to either reduce positivity (so that the capture of positively charged analytes is promoted). For instance, positively charged amino acids, such as R, H or K, may be substituted with negative, polar, or hydrophobic amino acids. Amino acids may be substituted with D, E, S, T, N, Q, G, A, V, L, I, P, C, F, Y or W. One or more modifications or mutations are preferably made to include polar, hydrophobic, positive, or negative in order to change the discrimination. Amino acids may be substituted with S, T, N, Q, G, A, V, L, I, P, C, R, K, D, E, F, Y, or W.

If the amino acid or polypeptide analyte is positively charged, the one or more modifications or mutations are preferably selected from the following specific substitutions:
R to D, E, S, T, N, Q, G, A, V, L, I, C, F, Y, or W,
K to D, E, S, T, N, Q, G, A, V, L, I, C, F, Y, or W,
D to S, T, N, Q, G, A, V, L, I, C, F, Y, or W, preferably D to N, Q, or A,
E to S, T, N, Q, G, A, V, L, I, C, F, Y, or W, preferably to E to N, Q, or A,
S to E, D, T, N, Q, A, V, L, or I, preferably S to E or D,
T to E, D, S, N, Q, A, V, L, or I, preferably T to E or D,
N to E, D, S, T, Q, A, V, L, or I preferably N to E or D, and
Q to E, D, S, T, N, A, V, L, or I, preferably Q to E or D.

If the amino acid or polypeptide analyte is positively charged, the variant preferably comprises a modification or mutation at one or more of the positions corresponding to R1, I15, D23, V24, L25, D127, L176 and E178 in SEQ ID NO: 2. The variant may comprise any number of modifications or mutations at these positions, such as 1, 2, 3, 4, 5, 6, 7, or 8 of these positions. The variant preferably comprises a modification or mutation at all of positions corresponding to R1, I15, D23, V24, L25, D127, L176 and E178 in SEQ ID NO: 2. The one or more modifications or mutations are preferably one or more substitutions. The amino acids at these positions may be mutated in any of the ways discussed above. The variant preferably comprises a modification or mutation at the position corresponding to D139 in SEQ ID NO: 2. The variant preferably comprises a modification or mutation at one or more of, such as all of, the positions corresponding to R1, I15, D23, V24, L25 and D139 in SEQ ID NO: 2. The variant preferably comprises a modification or mutation at one or more of, such as all of, the positions corresponding to D139, L176 and E178 in SEQ ID NO: 2. Any of these variants preferably further comprises a mutation or modification, preferably a substitution, at position D127. This is discussed in more detail below.

If the amino acid or polypeptide analyte is positively charged, the variant preferably comprises:
one or more of or all of R1D/I15F/D23Y/V24F/L25F/D139R/L176T/E178A,
one or more of or all of R1D/I15F/D23Y/V24F/L25F/D127R/D139R/L176T/E178A,
D217R,
D139R,
D127R/D139R,
one or more of or all of R1D/I15F/D23Y/V24F/L25F/D139R,
one or more of or all of R1D/I15F/D23Y/V24F/L25F/D127R/D139R,
one or more of or all of D139R/L176T/E178A,
one or more of or all of D127R/D139R/L176T/E178A,
one or more of or all of R1D/I15F/D23Y/V24F/L25F/D139N/L176T/E178A,
one or more of or all of R1D/I15F/D23Y/V24F/L25F/D127R/D139N/L176T/E178A,
D139N,
D127R/D139N,
one or more of or all of R1D/I15F/D23Y/V24F/L25F/D139N,
one or more of or all of R1D/I15F/D23Y/V24F/L25F/D127R/D139N,
one or more of or all of D139N/L176T/E178A, or
one or more of or all of D127R/D139N/L176T/E178A.

Examples of positively charged polypeptides and proteins are provided below.

If the amino acid or polypeptide analyte is negatively charged, one or more modifications or mutations are preferably made to include positive, polar, or hydrophobic amino acids in order to either reduce negativity (so that the capture of negatively charged analyte is promoted) or improve/stabilise the interaction with the polynucleotide binding protein (discussed in more detail below). For instance, negatively charged amino acids, such as D or E, may be substituted with positive, polar, or hydrophobic amino acids. Amino acids may be substituted with R, K, S, T, N, Q, G, A, V, L, I, P, C, F, Y or W. One or more modifications or mutations are preferably made to include polar, hydrophobic, positive, or negative in order to change the discrimination. Amino acids may be substituted with S, T, N, Q, G, A, V, L, I, P, C, R, K, D, E, F, Y, or W.

The variant preferably comprises one or more modifications or mutations which improve the ability of an actinoporin pore formed from the monomer to interact with a nucleotide or a polynucleotide. Nucleotides and polynucleotides are discussed in more detail below. The one or more modifications or mutations are preferably one or more additions, one or more deletions, one or more substitutions, or any combination thereof. The one or more modifications or mutations are preferably one or more substitutions.

Nucleotides and polynucleotides are typically negatively charged. One or more modifications or mutations are preferably made to include positive, polar, or hydrophobic amino acids in order to either reduce negativity (so that the capture of negatively charged analytes is promoted) or improve/stabilise the interaction with the polynucleotide binding protein. Amino acids may be substituted with R, K, S, T, N, Q, G, A, V, L, I, P, C, F, Y or W. One or more modifications or mutations are preferably made to include polar, hydrophobic, positive, or negative in order to change the discrimination. Amino acids may be substituted with S, T, N, Q, G, A, V, L, I, P, C, R, K, D, E, F, Y, or W.

For target nucleotides or polynucleotides, the one or more mutations are preferably selected from the following specific substitutions:
D to S, T, N, Q, A, V, L, I, F, R, or K,
E to S, T, N, Q, A, V, L, I, F, R, or K,
R to S, T, N, Q, A, V, L, I, or F,
K to S, T, N, Q, A, V, L, I, or F,
G to S, T, N, Q or A, V, L, I, or F,
A to S, T, N, Q or G, V, L, I, or F,
V to S, T, N, Q or G, A, L, I, or F,
L to S, T, N, Q or G, A, V, I, or F,
I to S, T, N, Q or G, A, V, L, or F,
S to T, N, Q, A, V, L, I, F, R, or K,
T to S, N, Q, A, V, L, I, F, R, or K,
N to S, T, Q, A, V, L, I, F, R, or K,
Q to S, T, N, A, V, L, I, F, R, or K, and
H to F, N, Q, or A.

For target nucleotides or polynucleotides, the variant preferably comprises a modification or mutation at one or more of the positions corresponding to positions E12, D23, D37, E39, E46, D57, E58, D66, D109, D127, D129, D131, D135, D139, E150, E172, E178 and E180 in SEQ ID NO: 2. The variant may comprise any number of modifications or mutations at these positions, such as 1, 2, 3, 4, 5, 6, 7, 8, 9, 10, 11, 12, 13, 14, 15, 16, 17, or 18 of these positions. The variant preferably comprises a modification or mutation at one or more of, such as all of, the positions corresponding to positions E12, D37, E39, E58, D66, E172 and E180 in SEQ ID NO: 2. The variant may comprise any number of modifications or mutations at these positions, such as 1, 2, 3, 4, 5, 6, or 7 of these positions. The variant preferably comprises a modification or mutation at one or more of, such as all of, the positions corresponding to positions D37, E39, E58, D66, E172 and E180 in SEQ ID NO: 2. These positions are in the pore entrance. The variant may comprise any number of modifications or mutations at these positions, such as 1, 2, 3, 4, 5 or 6 of these positions. The variant preferably comprises a modification or mutation at the position corresponding to position E12 in SEQ ID NO: 2. This position is in the transmembrane alpha helical region. The one or more modifications or mutations are preferably one or more additions, one or more deletions, one or more substitutions, or any combination thereof. The one or more modifications or mutations are preferably one or more substitutions.

The variant preferably comprises one or more modifications or mutations which alter the number of monomers which form an actinoporin pore. The one or more modifications or mutations are preferably one or more additions, one or more deletions, one or more substitutions, or any combination thereof. The variant preferably comprises one or more substitutions which alter the number of monomers which form an actinoporin pore comprising the monomer. The one or more modifications, mutations or substitutions preferably increase the number of monomers forming an actinoporin pore from 8 to 9. The variant preferably comprises a modification, such as an addition, deletion or substitution, or a substitution at the position corresponding to D127 in SEQ ID NO: 2. The variant preferably comprises D127R/N/K/R/S/Y/F/A or D127R/K at the corresponding position. The variant most preferably comprises D127R at the corresponding position. This substitution increases the number of monomers forming an actinoporin pore from mostly 8 to 8/9 in a 1:1 ratio (FIG. 3). These modifications, mutations or substitutions may be in addition to or instead of any of the modifications or mutations discussed above for improving the ability of an actinoporin pore to interact with a target analyte.

The variant is preferably a fragment lacking one or more amino acids from the N-terminus of SEQ ID NO: 2. This facilitates the insertion of the monomer into an artificial membrane. The fragment may lack any number of amino acids from the N-terminus of SEQ ID NO: 2 as long as the variant has at least about 54% identity to the sequence of SEQ ID NO: 2 over its entire length. The fragment preferably lacks up to about 32 amino acids from the N-terminus of SEQ ID NO: 2. The fragment may lack up to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 amino acids from the N-terminus of SEQ ID NO: 2.

The actinoporin monomers of the invention are preferably capable of inserting into an artificial membrane. The variant preferably comprises one or more modifications which facilitate insertion of the actinoporin monomer into an artificial membrane. Insertion can be measured using routine methods, including any of those described in WO 2016/034591, WO 2017/149316, WO 2017/149317, WO 2017/149318, WO 2018/211241, and WO 2019/002893 (all incorporated by reference herein in their entirety) and in the Examples.

The variant is preferably a fragment lacking one or more amino acids from the C-terminus of SEQ ID NO: 2. The fragment may lack any number of amino acids from the C-terminus of SEQ ID NO: 2 as long as the variant has at least about 54% identity to the sequence of SEQ ID NO: 2 over its entire length. The fragment preferably lacks up to about 10 amino acids from the C-terminus of SEQ ID NO: 2. The fragment may lack up to about 1, 2, 3, 4, 5, 6, 7, 8, 9, or 10 amino acids from the C-terminus of SEQ ID NO: 2. The fragment preferably lacks up to or at least about 5 amino acids from the C-terminus of SEQ ID NO: 2. The fragment preferably lacks about 5 amino acids from the C-terminus of SEQ ID NO: 2. The fragment may include any of the modifications or mutations discussed above for improving the ability of an actinoporin pore to interact with a target analyte and/or altering the number of monomers which form an actinoporin pore. Fragments in accordance with invention may lack one or more amino acids from the N-terminus of SEQ ID NO: 2 and one or more amino acids from the C-terminus of SEQ ID NO: 2.

Deletion of one or more amino acids from the C-terminus of SEQ ID NO: 2, such as about 5 amino acids from the C-terminus of SEQ ID NO: 2, is also capable in combination with a modification or mutation at the position corresponding to D127 in SEQ ID NO: 2 of increasing the number of monomers which form an actinoporin pore. Variants including a mutation or modification, such as a substitution, at the position corresponding to position D127 in SEQ ID NO: 2, such as D127R, and lacking 5 amino acids from the C-terminus of SEQ ID NO: 2 are capable of forming 9-mer and 10-mer actinoporin pore oligomers (FIG. 7).

The actinoporin monomer typically retains the ability to form the same 3D structure as the wild type actinoporin monomer, such as the same 3D structure as an actinoporin monomer having the sequence of SEQ ID NO: 2. Any number of modifications or mutations may be made in addition to the modifications or mutations described herein provided that the actinoporin monomer retains the improved properties imparted on it by the modifications or mutations of the present invention.

Typically, the actinoporin monomer will retain the ability to form a structure comprising an alpha helix. Therefore, it is envisaged that further mutations may be made without affecting the ability of the monomer to form an actinoporin pore that can translocate analytes. It is also expected that deletions of one or more amino acids can be made in any of the loop regions and/or in the N-terminal and/or C-terminal regions of the actinoporin monomer without affecting the ability of the monomer to form a pore that can translocate analytes.

Amino acid substitutions may be made to the amino acid sequence of SEQ ID NO: 2 in addition to those discussed above, for example up to 1, 2, 3, 4, 5, 10, 20 or 30 substitutions. Conservative substitutions replace amino acids with other amino acids of similar chemical structure, similar chemical properties, or similar side-chain volume. The amino acids introduced may have similar polarity, hydrophilicity, hydrophobicity, basicity, acidity, neutrality, or charge to the amino acids they replace. Alternatively, the conservative substitution may introduce another amino acid that is aromatic or aliphatic in the place of a pre-existing aromatic or aliphatic amino acid. Conservative amino acid changes are well-known in the art.

The actinoporin monomer may be modified to introduce one or more cysteines, one or more hydrophobic amino acids, one or more charged amino acids, one or more non-native amino acids, one or more polar amino acids, or one or more photoreactive amino acids. Any number and combination of such introductions may be made. The introduction is preferably by substitution or addition.

One or more amino acid residues of the amino acid sequence of SEQ ID NO: 2 may additionally be deleted from the polypeptides described above. Up to 1, 2, 3, 4, 5, 10, 20 or 30 or more residues may be deleted.

One or more amino acids may be alternatively or additionally added to the polypeptides described above. An extension may be provided at the amino terminal or carboxy terminal of the amino acid sequence of SEQ ID NO: 2 or variant or fragment thereof. The extension may be quite short, for example from 1 to 10 amino acids in length. Alternatively, the extension may be longer, for example up to 50 or 100 amino acids. A carrier protein may be fused to an amino acid sequence according to the invention. Other fusion proteins are discussed in more detail below.

The one or more modifications in the actinoporin monomer preferably improve the ability of an actinoporin pore comprising the actinoporin monomer to characterise an analyte. For example, modifications, mutations/substitutions are contemplated to alter the number, size, shape, placement, or orientation of the constriction within a channel from the actinoporin monomer of the invention. The actinoporin monomer or the variant of SEQ ID NO: 2 may have any of the particular modifications or substitutions disclosed in WO 2016/034591, WO 2017/149316, WO 2017/149317, WO 2017/149318, WO 2018/211241, and WO 2019/002893 (all incorporated by reference herein in their entirety).

Any actinoporin monomer and/or variant based on SEQ ID NO: 2 described herein preferably further comprises the sequence GHM or the amino acid S at its N-terminus.

### Constructs

The invention also provides a construct comprising two or more covalently attached actinoporin monomers of the invention. The construct may comprise 2 or more, 3 or more, 4 or more, 5 or more, 6 or more, 7 or more, 8 or more, 9 or more or 10 or more actinoporin monomers of the invention. The construct may comprise at least 2, at least 3, at least 4, at least 5, at least 6, at least 7, at least 8, at least 9 or at least 10 actinoporin monomers of the invention. The two or more actinoporin monomers may be the same or different. The two or more actinoporin monomers may differ based on one or more of (a) the sequence of the actinoporin monomer, (b) the linker, and (c) the attachment position on the actinoporin monomer. The actinoporin monomers may differ based on (a), (b), (c), (a) and (b), (a) and (c), (b) and (c), or (a), (b) and (c). The two or more actinoporin monomers are preferably the same (i.e., identical).

The construct preferably comprises two actinoporin monomers. The two or more actinoporin monomers may be the same or different. The two or more actinoporin monomers are preferably the same (i.e., identical).

The actinoporin monomers may be genetically fused, optionally via a linker, or chemically fused, for instance via a chemical crosslinker. Methods for covalently attaching monomers are disclosed in WO 2017/149316, WO 2017/149317, and WO 2017/149318 (incorporated herein by reference in their entirety).

The linker is preferably an amino acid sequence and/or a chemical crosslinker. Suitable amino acid linkers, such as peptide linkers, are known in the art. The length, flexibility and hydrophilicity of the amino acid or peptide linker are typically designed such that the actinoporin monomers in the construct are in the correct orientation to form an actinoporin pore. Flexible and rigid linkers that are useful in the constructs are discussed above.

Suitable chemical crosslinkers are well-known in the art. Suitable chemical crosslinkers include, but are not limited to, those including the following functional groups: maleimide, active esters, succinimide, azide, alkyne (such as dibenzocyclooctynol (DIBO or DBCO), difluoro cycloalkynes and linear alkynes), phosphine (such as those used in traceless and non-traceless Staudinger ligations), haloacetyl (such as iodoacetamide), phosgene type reagents, sulfonyl chloride reagents, isothiocyanates, acyl halides, hydrazines, disulfides, vinyl sulfones, aziridines and photoreactive reagents (such as aryl azides, diaziridines).

Reactions between amino acids and functional groups may be spontaneous, such as cysteine/maleimide, or may require external reagents, such as Cu(I) for linking azide and linear alkynes.

Linkers can comprise any molecule that stretches across the distance required. Linkers can vary in length from one carbon (phosgene-type linkers) to many Angstroms. Examples of linker molecules, include but are not limited to, are polyethyleneglycols (PEGs), polypeptides, polysaccharides, deoxyribonucleic acid (DNA), peptide nucleic acid (PNA), threose nucleic acid (TNA), glycerol nucleic acid (GNA), saturated and unsaturated hydrocarbons, polyamides. These linkers may be inert or reactive, in particular they may be chemically cleavable at a defined position, or may be themselves modified with a fluorophore or ligand. The linker is preferably resistant to reducing agents, such as dithiothreitol (DTT), following the covalent attachment.

Crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate, di-maleimide PEG 1k, di-maleimide PEG 3.4k, di-maleimide PEG 5k, di-maleimide PEG 10k, bis(maleimido)ethane (BMOE), bis-maleimidohexane (BMH), 1,4-bis-maleimidobutane (BMB), 1,4 bis-maleimidyl-2,3-dihydroxybutane (BMDB), BM[PEO]2 (1,8-bis-maleimidodiethyleneglycol), BM[PEO]3 (1,11-bis-maleimidotriethylene glycol), tris[2-maleimidoethyl]amine (TMEA), DTME dithiobismaleimidoethane, bis-maleimide PEG3, bis-maleimide PEG11, DBCO-maleimide, DBCO-PEG4-maleimide, DBCO-PEG4-NH2, DBCO-PEG4-NHS, DBCO-NHS, DBCO-PEG-DBCO 2.8 kDa, DBCO-PEG-DBCO 4.0 kDa, DBCO-15 atoms-DBCO, DBCO-26 atoms-DBCO, DBCO-35 atoms-DBCO, DBCO-PEG4-S-S-PEG3-biotin, DBCO-S-S-PEG3-biotin, DBCO-S-S-PEG11-biotin, (succinimidyl 3-(2-pyridyldithio)propionate (SPDP) and maleimide-PEG(2 kDa)-maleimide (ALPHA,OMEGA-BIS-MALEIMIDO POLY(ETHYLENE GLYCOL)). One crosslinker is maleimide-propyl-SRDFWRS-(1,2-diaminoethane)-propyl-maleimide.

The linker is preferably resistant to dithiothreitol (DTT). Suitable linkers include, but are not limited to, iodoacetamide-based and maleimide-based linkers.

The actinoporin monomers may be connected using two or more linkers each comprising a hybridisable region and a group capable of forming a covalent bond. The hybridisable regions in the linkers hybridize and link the actinoporin monomers. The linked actinoporin monomers are then coupled via the formation of covalent bonds between the groups. Any of the specific linkers disclosed in WO 2010/086602 (incorporated herein by reference in its entirety) may be used in accordance with the invention.

The linkers may be labelled. Suitable labels include, but are not limited to, fluorescent molecules (such as Cy3 or AlexaFluor®555), radioisotopes, e.g. ¹²⁵I, ³⁵S, ³²P, enzymes, antibodies, antigens, polynucleotides and ligands such as biotin. Such labels allow the amount of linker to be quantified. The label could also be a cleavable purification tag, such as biotin, or a specific sequence to show up in an identification method, such as a peptide that is not present in the protein itself, but that is released by trypsin digestion.

A method of connecting the actinoporin monomers is via cysteine linkage. This can be mediated by a bi-functional chemical crosslinker or by an amino acid linker with a terminal presented cysteine residue.

Another method of attachment via 4-azidophenylalanine or Faz linkage. This can be mediated by a bi-functional chemical linker or by a polypeptide linker with a terminal presented 4-azidophenylalanine or Faz residue. Additional suitable linkers are discussed in more detail below.

### Actinoporin Pores of the Invention

The terms “actinoporin pore”, “actinoporin pore oligomer” and “actinoporin pore complex”, as used interchangeably herein, refer to an oligomeric actinoporin pore comprising at least one actinoporin monomer of the invention (including, e.g., one or more actinoporin monomers such as two or more actinoporin monomers, three or more actinoporin monomers etc.). The actinoporin pore of the invention has the features of a biological pore, i.e., it has a typical protein structure and defines a channel. When the actinoporin pore is provided in an environment having membrane components, membranes, cells, or an insulating layer, the actinoporin pore will insert in the membrane or the insulating layer and form a “transmembrane actinoporin pore”.

The actinoporin pore preferably has or comprises any of the structures discussed above with the actinoporin monomers of the invention. The actinoporin pore of the invention includes an actinoporin pore with two constrictions, i.e., two channel constrictions positioned in such a way that one constriction does not interfere in the accuracy of the other constriction. The skilled person is capable of creating multiple constrictions in pores, for instance using an ancillary peptide attached, preferably covalently attached, to each actinoporin monomer in the actinoporin pore. These types of modifications are described in WO 2016/034591, WO 2017/149316, WO 2017/149317, WO 2019/002893, WO 2017/149318, WO 2018/211241, and WO 2019/002893 (herein all incorporated by reference in their entirety). The actinoporin pore of the invention includes an actinoporin pore with one constriction.

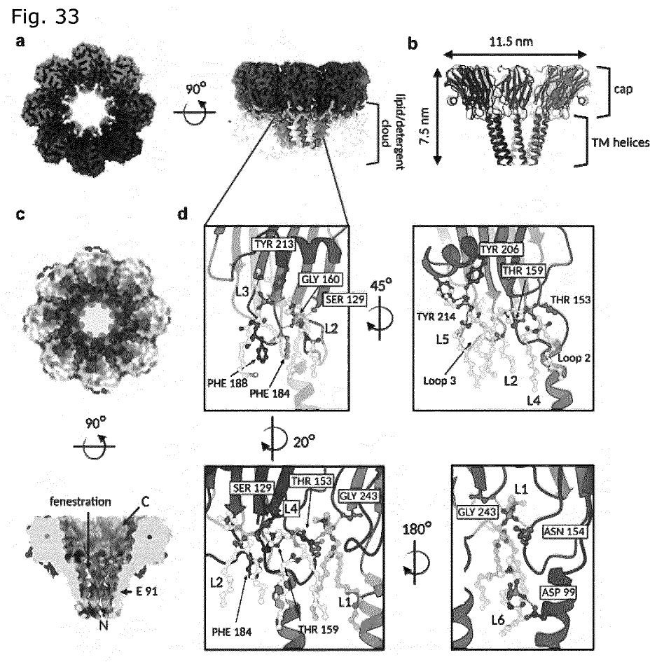

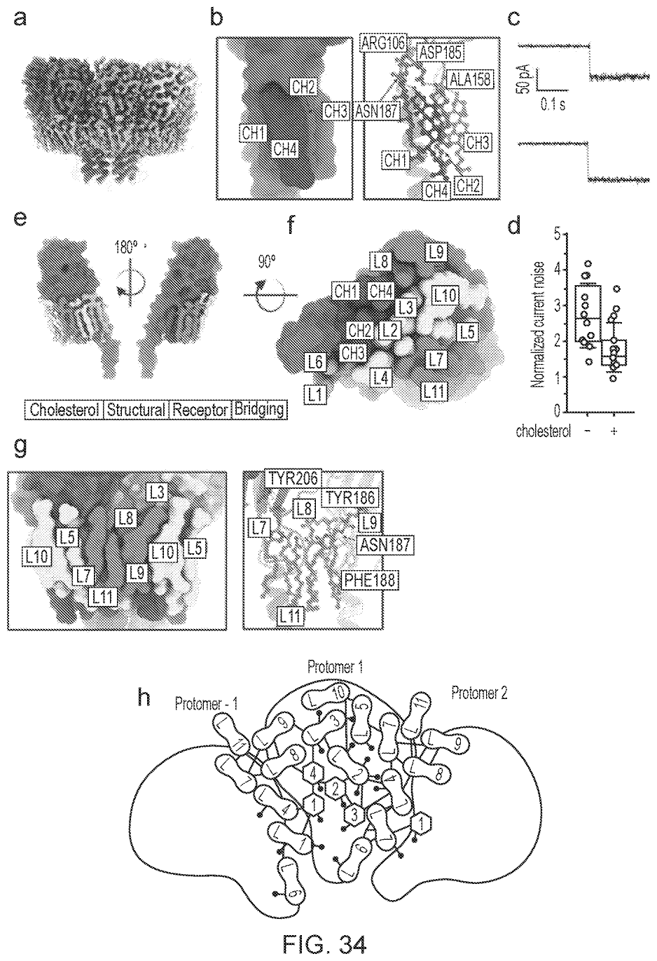

The actinoporin pore may be any size but preferably has the dimensions of the wild-type actinoporin pore (FIG. 33). The actinoporin pore formed from monomer preferably comprises a cap region and a transmembrane alpha helical region.

The actinoporin pore preferably has an external diameter of from about 6.0 to about 15.0 nm at its widest point, such as from about 8.0 to about 13.0 nm or from about 9.0 to about 12.0 nm at its widest point. The actinoporin pore preferably has an external diameter of about 10.5 nm or 11.5 nm (8mer and 9mer respectively) at its widest point. The actinoporin pore preferably has a total length of from about 3.0 to about 15.0 nm, such as from about 5.0 to about 9.0 nm or from about 6.0 to about 8.0 nm. The actinoporin pore preferably has a total length of about 7.0 nm. References to “total length” and “length” relate to the length of the pore or pore region when viewed from the side (see, e.g., the side view in FIG. 33b).

The cap region preferably has a length of from about 2.0 to about 6.0 nm, such as from about 3.0 to about 5.0 nm or from about 3.5 to about 4.5 nm. The cap region preferably has a length of about 4.0 nm. The channel defined by the cap region preferably has an opening of from about 4.0 to about 10.0 nm in diameter, such as from about 5.0 to about 9.0 nm or from about 6.0 to about 8.0 nm in diameter. The channel defined by the cap region preferably has an opening of about 6.8 nm or 7.7 nm in diameter (8mer and 9mer respectively).

The transmembrane alpha helical region preferably has a length of from about 2.0 to about 11.0 nm, such as from about 2.5 to about 8.0 nm, from about 2.7 to about 5.0 nm, or from about 3.0 to about 4.0 nm. The transmembrane alpha helical region preferably has a length of about 3.5 nm. The transmembrane alpha helical region preferably has a length of about 3.5 nm. The channel defined by the transmembrane alpha helical region is preferably from about 0.5 to about 4.5 nm in diameter at its narrowest point, such as from about 1.0 to about 3.5 nm or from about 1.5 to about 3.0 nm in diameter at its narrowest point. The channel defined by the transmembrane alpha helical region is preferably about 2.2 nm or about 2.8 nm in diameter (8mer and 9mer respectively) at its narrowest point. The narrowest point typically forms the constriction.

All of the measurements above are based on measuring from backbone to backbone of the amino acids forming the different regions (whereas FIG. 33 measures from Van der Waals surface to Van der Waals surface).

The invention provides an actinoporin pore comprising at least one actinoporin monomer of the invention. The actinoporin pore typically comprises at least 6, 7, 8, 9 or 10 actinoporin monomers of the invention. The actinoporin pore preferably comprises 7, 8, 9 or 10 actinoporin monomers of the invention. The actinoporin monomers are typically the same (i.e., identical). The greater the number of actinoporin monomers in the actinoporin pore oligomer, the larger the pore formed by the actinoporin pore oligomer. Larger pores allow larger analytes to be detected or characterised.

The actinoporin pore is preferably a homooligomer comprising 6 to 10, such as 6, 7, 8, 9 or 10, actinoporin monomers of the invention. The actinoporin monomers are typically identical. The actinoporin pore preferably comprises 8, 9 or identical actinoporin monomers of the invention. The actinoporin pore preferably comprises 8 or 9 identical actinoporin monomers of the invention. The actinoporin pore preferably comprises 8 identical actinoporin monomers of the invention. The actinoporin pore preferably comprises 9 identical actinoporin monomers of the invention. The actinoporin monomers may be any of those discussed above.

The invention provides an actinoporin pore comprising at least one construct of the invention. The actinoporin pore typically comprises at least 1, 2, 3, 4 or 5 constructs of the invention. The actinoporin pore comprises sufficient actinoporin monomers to form a pore. For instance, an octameric actinoporin pore may comprise (a) four constructs each comprising two actinoporin monomers, (b) two constructs each comprising four actinoporin monomers, (c) one construct comprising two actinoporin monomers and six actinoporin monomers that do not form part of a construct, (d) three constructs comprising two actinoporin monomers and two actinoporin monomers that do not form part of a construct, and (e) combinations thereof. Same and additional possibilities are provided for a nonameric actinoporin pore or decameric actinoporin pore for instance. Other combinations of constructs and monomers can be envisaged by the skilled person. One or more constructs of the invention may be used to form an actinoporin pore for characterising, such as sequencing, analytes. The actinoporin pore preferably comprises 4 constructs of the invention each of which comprises two actinoporin monomers. The constructs are typically the same (i.e., identical).

The actinoporin pore is preferably a homooligomer comprising 1-5, such as 1, 2, 3, 4, 5, constructs of the invention. The constructs are typically the same (i.e., identical). The actinoporin pore preferably comprises 4 identical constructs of the invention each of which comprises two actinoporin monomers. The constructs may be any of those discussed above.

The actinoporin monomers in the actinoporin pore are preferably all approximately the same length or are the same length. The helical regions of the actinoporin monomers of the invention in the pore are preferably approximately the same length or are the same length. Length may be measured in number of amino acids and/or units of length.

The actinoporin pore of the invention may be isolated, substantially isolated, purified, or substantially purified. An actinoporin pore of the invention is isolated or purified if it is completely free of any other components, such as lipids or other pores. An actinoporin pore is substantially isolated if it is mixed with carriers or diluents which will not interfere with its intended use. For instance, an actinoporin pore is substantially isolated or substantially purified if it is present in a form that comprises less than 10%, less than 5%, less than 2% or less than 1% of other components, such as block copolymers, lipids or other pores. Alternatively, an actinoporin pore of the invention may be present in a membrane. Suitable membranes are discussed below.

An actinoporin pore of the invention may be present as an individual or single actinoporin pore. Alternatively, an actinoporin pore of the invention may be present in a homologous or heterologous population of two or more actinoporin pores or pores. For instance, the actinoporin pore may be present in a heterologous population of two or more actinoporin pores formed from different numbers of monomers. Other formats involving the actinoporin pores of the invention are discussed in more detail below.

### Multimeric Actinoporin Pores

The invention also provides a pore multimer comprising two or more pores, wherein at least one of the pores is an actinoporin pore of the invention. The multimer may comprise any number of pores, such as 3, 4, 5, 6, 7 or 8 or more pores. Any number of the pores in the multimer, including all of them, may be an actinoporin pore of the invention.

The pore multimer may be a double actinoporin pore comprising a first actinoporin pore of the invention and a second actinoporin pore. Both the first actinoporin pore and the second actinoporin pore are preferably actinoporin pores of the invention. In the double actinoporin pore, the first actinoporin pore may be attached to the second actinoporin pore by hydrophobic interactions and/or by one or more disulfide bonds. One or more, such as 2, 3, 4, 5, 6, 8, 9, for example all, of the monomers in the first actinoporin pore and/or the second actinoporin pore may be modified to enhance such interactions. This may be achieved in any suitable way. Particular methods of forming double pores are described in WO 2019/002893 (incorporated by reference herein in its entirety).

The pore multimer of the invention may be isolated, substantially isolated, purified, or substantially purified. Such terms are defined above with reference to the actinoporin pores of the invention.

The pore multimer of the invention may be used in any of the membranes, methods, uses, kits, apparatuses, arrays, or systems of the invention described below.

### Membrane Embodiments

The invention also provides an actinoporin pore of the invention or a pore multimer of the invention which is comprised in a membrane. The membrane is preferably an artificial membrane. Artificial has its normal meaning in the art. An artificial membrane is a membrane that does not appear in nature. The skilled person is capable of determining whether or not a membrane is naturally occurring or artificial. Suitable artificial membranes are discussed in more detail below.

The invention also provides an artificial membrane comprising an actinoporin pore derived from Orbicella faveolata. The actinoporin pore may be any actinoporin pore derived from Orbicella faveolata, including wild type actinoporin pores. The actinoporin pore may comprise the sequence shown in SEQ ID NO: 1. The actinoporin pore is preferably an actinoporin pore of the invention or a pore multimer of the invention. These products are directly applicable for use in molecular sensing, such as analyte characterisation and sequencing. Suitable membranes are discussed in more detail below.

### Method for Making Modified Proteins

Methods for introducing or substituting non-naturally occurring amino acids in actinoporin monomers are also well known in the art and described in WO 2019/002893 (incorporated by reference herein in its entirety). The proteins may be modified to assist their identification or purification, for example by the addition of a streptavidin tag or by the addition of a signal sequence to promote their secretion from a cell where the monomer does not naturally contain such a sequence. The proteins may also be produced using D-amino acids or a mixture of L-amino acids and D-amino acids. This is conventional in the art for producing such proteins or peptides.

The actinoporin monomer or any protein described herein may be chemically modified. The protein can be chemically modified in any way and at any site. The protein may be chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The protein may be chemically modified by the attachment of any molecule, such as a dye or a fluorophore.

The protein may be chemically modified with a molecular adaptor that facilitates the interaction between a pore comprising the monomer and a target nucleotide or target polynucleotide sequence. Suitable adaptors, including a cyclic molecule, a cyclodextrin, a species that is capable of hybridization, a DNA binder or interchelator, a peptide or peptide analogue, a synthetic polymer, an aromatic planar molecule, a small positively charged molecule or a small molecule capable of hydrogen-bonding, are described in WO 2019/002893 (incorporated by reference herein in its entirety). The molecular adaptor may be attached using any of the methods and linkers discussed above.

The protein, such as the actinoporin monomer of the invention, may be attached to a polynucleotide binding protein. This forms a modular sequencing system that may be used in the methods of sequencing of the invention. Polynucleotide binding proteins are polymerases, exonucleases, helicases, and topoisomerases, such as gyrases. Suitable polynucleotide binding proteins include, but are not limited to, exonuclease I from E. coli, exonuclease III enzyme from E. coli, RecJ from T. thermophilus and bacteriophage lambda exonuclease, TatD exonuclease and variants thereof. Three subunits comprising the RecJ sequence from T. thermophilus or a variant thereof interact to form a trimer exonuclease. The polymerase may be PyroPhage® 3173 DNA Polymerase (which is commercially available from Lucigen® Corporation), SD Polymerase (commercially available from Bioron®) or variants thereof. The polynucleotide binding protein may be Phi29 DNA polymerase or a variant thereof. The topoisomerase is preferably a member of any of the Moiety Classification (EC) groups 5.99.1.2 and 5.99.1.3.

The polynucleotide binding proteins is most preferably derived from a helicase, such as Hel308 Mbu, Hel308 Csy, Hel308 Tga, Hel308 Mhu, TraI Eco, XPD Mbu or a variant thereof. Any helicase may be used in the invention. The helicase may be or be derived from a Hel308 helicase, a RecD helicase, such as TraI helicase or a TrwC helicase, a XPD helicase or a Dda helicase. The helicase may be any of the helicases, modified helicases or helicase constructs disclosed in WO 2013/057495; WO 2013/098562; WO 2013098561; WO 2014/013260; WO 2014/013259; WO 2014/013262 and WO 2015/055981. All of these are incorporated by reference herein in their entirety.

The polynucleotide binding protein can be covalently attached to the monomer using any method known in the art. The monomer and protein may be chemically fused or genetically fused. Genetic fusion of a monomer to a polynucleotide binding protein is discussed in WO 2010/004265 (incorporated herein by reference in its entirety). The polynucleotide binding protein may be attached via cysteine linkage using any method described above.

The polynucleotide binding protein may be attached directly to the protein via one or more linkers. The molecule may be attached to the actinoporin monomer using the hybridization linkers described in as WO 2010/086602 (incorporated herein by reference in its entirety). Alternatively, peptide linkers may be used. Suitable peptide linkers are discussed above.

Any of the proteins may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the protein. An example of this would be to react a gel-shift reagent to a cysteine engineered on the outside of the protein. This has been demonstrated as a method for separating hemolysin hetero-oligomers (Chem Biol. 1997 July; 4(7):497-505).

Any of the proteins may be labelled with a revealing label. The revealing label may be any suitable label which allows the protein to be detected. Suitable labels include, but are not limited to, fluorescent molecules, radioisotopes, e.g., 125I, 35S, enzymes, antibodies, antigens, polynucleotides, and ligands such as biotin.

The protein may also contain other non-specific modifications as long as they do not interfere with the function of the protein. A number of non-specific side chain modifications are known in the art and may be made to the side chains of the protein(s). Such modifications include, for example, reductive alkylation of amino acids by reaction with an aldehyde followed by reduction with NaBH4, amidation with methylacetimidate or acylation with acetic anhydride.

Any of the proteins can be produced using standard methods known in the art. Polynucleotide sequences encoding a protein may be derived and replicated using standard methods in the art. Polynucleotide sequences encoding a protein may be expressed in a bacterial host cell using standard techniques in the art. The protein may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

Proteins may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system, and the Gilson HPLC system.

### Method for Producing Actinoporin Monomers

The invention provides methods for producing an actinoporin monomer of the invention. The method comprises modifying or mutating the sequence shown in SEQ ID NO: 1 or SEQ ID NO: 2. Any of the modifications or mutations discussed above may be carried out.

Alternatively, the method comprises expressing a polynucleotide of the invention in a host cell or producing the actinoporin monomer using in vitro translation and transcription (IVTT). Such methods are described in more detail below.

Any of the embodiments discussed above with reference to the actinoporin monomers of the invention equally applies to these methods.

### Method of Producing Pores

The invention also provides methods for producing an actinoporin pore of the invention or a pore multimer of the invention.

The method may involve expressing the actinoporin pore in a host cell. In particular, the method may comprise expressing at least one actinoporin monomer of the invention or a construct of the invention and sufficient actinoporin monomers or constructs to form the actinoporin pore or the pore multimer in a host cell and allowing the actinoporin pore or pore multimer to form in the host cell. The sufficient actinoporin monomers or constructs are preferably sufficient actinoporin monomers of the invention or sufficient constructs of the invention. The numbers of actinoporin monomers or constructs needed to form the actinoporin pores of the invention or pore multimers of the invention are discussed above. Suitable host cells and expression systems are known in the art and are discussed in the Example.

The method may involve forming the actinoporin pore in a non-cellular or in vitro context. In particular, the method may comprise contacting at least one actinoporin monomer of the invention or a construct of the invention with sufficient actinoporin monomers or constructs in vitro and allowing the formation of the actinoporin pore or pore multimer. The actinoporin monomer or the construct may be produced separately by in vitro translation and transcription (IVTT) and then incubated with the sufficient actinoporin monomers or constructs. The sufficient actinoporin monomers or constructs are preferably sufficient actinoporin monomers of the invention or sufficient constructs of the invention. The numbers of actinoporin monomers or constructs needed to form the actinoporin pores of the invention or pore multimers of the invention are discussed above. The method may be conducted in an “in vitro system”, which refers to a system comprising at least the necessary components and environment to execute said method, and makes use of biological molecules, organisms, a cell (or part of a cell) outside of their normal naturally occurring environment, permitting a more detailed, more convenient, or more efficient analysis than can be done with whole organisms. An in vitro system may also comprise a suitable buffer composition provided in a test tube, wherein said protein components to form the complex have been added. A person skilled in the art is aware of the options to provide said system.

Some or all of the components of the actinoporin monomer, actinoporin pore or pore multimer may be tagged to facilitate purification. Purification can also be performed when the components are untagged. Methods known in the art (e.g., ion exchange, gel filtration, hydrophobic interaction column chromatography etc.) can be used alone or in different combinations to purify the components of the pore.

The actinoporin pore or pore multimer can be made prior to insertion into a membrane or after insertion of the components into a membrane.

Methods for making the pores and complexes of the invention and ways of tagging them are disclosed in WO 2016/034591, WO 2017/149316, WO 2017/149317 and, WO 2017/149318, WO 2018/211241, and WO 2019/002893 (all incorporated by reference herein in their entirety).

### Methods of Characterising an Analyte

The invention provides a method of determining the presence, absence or one or more characteristics of a target analyte. The method involves contacting the target analyte with an actinoporin pore of the invention, a pore multimer of the invention or a membrane of the invention and taking one or more measurements as the target analyte moves with respect to the actinoporin pore, pore multimer or membrane and thereby determining the presence, absence or one or more characteristics of the target analyte.

The invention also provides a method of determining the presence, absence or one or more characteristics of a target analyte. The method involves contacting the target analyte with an actinoporin pore of the invention, a pore multimer of the invention or a membrane of the invention such that the target analyte moves with respect to, such as into or through, the actinoporin pore, pore multimer or membrane and taking one or more measurements as the target analyte moves with respect to the actinoporin pore or pore multimer and thereby determining the presence, absence or one or more characteristics of the target analyte.

The target analyte may also be called the template analyte or the analyte of interest. The actinoporin pore of the invention, the pore multimer of the invention or the membrane of the invention may be any of those discussed above.

The method is for determining the presence, absence or one or more characteristics of a target analyte. The method may be for determining the presence, absence or one or more characteristics of at least one target analyte. The method may concern determining the presence, absence or one or more characteristics of two or more target analytes. The method may comprise determining the presence, absence or one or more characteristics of any number of target analytes, such as 2, 5, 10, 15, 20, 30, 40, 50, 100 or more analytes. Any number of characteristics of the one or more target analytes may be determined, such as 1, 2, 3, 4, 5, 10 or more characteristics.

The binding of a molecule in the channel of the actinoporin pore or pore multimer, or in the vicinity of either opening of the channel will have an effect on the open-channel ion flow through the actinoporin pore or pore multimer, which is the essence of “molecular sensing”. In a similar manner to the nucleic acid sequencing application, variation in the open-channel ion flow can be measured using suitable measurement techniques by the change in electrical current (for example, WO 2000/28312 and D. Stoddart et al., Proc. Natl. Acad. Sci., 2010, 106, 7702-7 or WO 2009/077734; all incorporated herein by reference in their entirety). The degree of reduction in ion flow, as measured by the reduction in electrical current, is related to the size of the obstruction within, or in the vicinity of, the pore. Binding of a molecule of interest, also referred to as an “analyte”, in or near the pore therefore provides a detectable and measurable event, thereby forming the basis of a “biological sensor”. Suitable molecules for nanopore sensing include nucleic acids; proteins; peptides; polysaccharides and small molecules (refers here to a low molecular weight (e.g., <900 Da or <500 Da) organic or inorganic compound) such as pharmaceuticals, toxins, cytokines, and pollutants. Detecting the presence of biological molecules finds application in personalised drug development, medicine, diagnostics, life science research, environmental monitoring and in the security and/or the defence industry.

The actinoporin pore or pore multimer may serve as a molecular or biological sensor. The target analyte molecule that is to be detected may bind to either face of the channel, or within the lumen of the channel itself. The position of binding may be determined by the size of the molecule to be sensed.

The target analyte preferably comprises or consists of a metal ion, an inorganic salt, a polymer, an amino acid, a peptide, a polypeptide, a protein, a nucleotide, an oligonucleotide, a polynucleotide, a polynucleotide-polypeptide conjugate, a monosaccharide, an oligosaccharide, a polysaccharide, a dye, a bleach, a pharmaceutical, a diagnostic agent, a recreational drug, an explosive, a toxic compound, or an environmental pollutant. The target analyte preferably comprises or consists of a polypeptide, a protein, an oligonucleotide, a polynucleotide, a polynucleotide-polypeptide conjugate, an oligosaccharide, or a polysaccharide. The target analyte may comprise two or more different molecules, such as a peptide and a polypeptide. The target analyte may be a polynucleotide-polypeptide conjugate. The target analyte may be a biomarker. The method may concern determining the presence, absence or one or more characteristics of two or more target analytes of the same type, such as two or more proteins, two or more nucleotides or two or more pharmaceuticals. Alternatively, the method may concern determining the presence, absence or one or more characteristics of two or more target analytes of different types, such as one or more proteins, one or more nucleotides and one or more pharmaceuticals.

The target analyte can be secreted from cells. Alternatively, the target analyte can be an analyte that is present inside cells such that the target analyte must be extracted from the cells before the method can be carried out. The target analyte may be obtained from or extracted from any organism or microorganism. The target analyte may be obtained from a human or animal, e.g., from urine, lymph, saliva, mucus, seminal fluid, or amniotic fluid, or from whole blood, plasma, or serum. The target analyte may be obtained from a plant e.g., a cereal, legume, fruit, or vegetable.

The actinoporin pore or pore multimer may be modified via recombinant or chemical methods to increase the strength of binding, the position of binding, or the specificity of binding of the molecule to be sensed. Typical modifications include addition of a specific binding moiety complimentary to the structure of the molecule to be sensed. Where the analyte molecule comprises a nucleic acid, this binding moiety may comprise a cyclodextrin or an oligonucleotide; for small molecules this may be a known complimentary binding region, for example the antigen binding portion of an antibody or of a non-antibody molecule, including a single chain variable fragment (scFv) region or an antigen recognition domain from a T-cell receptor (TCR); or for proteins, it may be a known ligand of the target protein. In this way the actinoporin pore or pore multimer may be rendered capable of acting as a molecular sensor for detecting presence in a sample of suitable antigens (including epitopes) that may include cell surface antigens, including receptors, markers of solid tumours or haematologic cancer cells (e.g. lymphoma or leukaemia), viral antigens, bacterial antigens, protozoal antigens, allergens, allergy related molecules, albumin (e.g. human, rodent, or bovine), fluorescent molecules (including fluorescein), blood group antigens, small molecules, drugs, enzymes, catalytic sites of enzymes or enzyme substrates, and transition state analogues of enzyme substrates. As described above, modifications may be achieved using known genetic engineering and recombinant DNA techniques. The positioning of any adaptation would be dependent on the nature of the molecule to be sensed, for example, the size, three-dimensional structure, and its biochemical nature. The choice of adapted structure may make use of computational structural design. Determination and optimization of protein-protein interactions or protein-small molecule interactions can be investigated using technologies such as a BIAcore® which detects molecular interactions using surface plasmon resonance (BIAcore, Inc., Piscataway, NJ; see also www.biacore.com).

The target analyte preferably comprises or consists of an amino acid, a peptide, a polypeptides, or protein. The amino acid, peptide, polypeptide, or protein can be naturally occurring or non-naturally occurring. The polypeptide or protein can include within them synthetic or modified amino acids. Several different types of modification to amino acids are known in the art. Suitable amino acids and modifications thereof are above. It is to be understood that the target analyte can be modified by any method available in the art.

The target analyte preferably comprises a polypeptide. The term polypeptide is interchangeable with protein. Any suitable polypeptide can be characterised. The polypeptide may be an unmodified protein or a portion thereof, or a naturally occurring polypeptide or a portion thereof. The target polypeptide may be secreted from cells. Alternatively, the target polypeptide can be produced inside cells such that it must be extracted from cells for characterisation. The target polypeptide may be a biomarker polypeptide. The polypeptide may comprise the products of cellular expression of a plasmid, e.g., a plasmid used in cloning of proteins in accordance with the methods described in Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016).

The polypeptide can be provided as an impure mixture of one or more polypeptides and one or more impurities. Impurities may comprise truncated forms of the target polypeptide which are distinct from the “target polypeptides” for characterisation. For example, the target polypeptide may be a full-length protein and impurities may comprise fractions of the protein. Impurities may also comprise proteins other than the target protein, e.g., which may be co-purified from a cell culture or obtained from a sample.

A polypeptide may comprise any combination of any amino acids, amino acid analogs and modified amino acids (i.e., amino acid derivatives). Amino acids (and derivatives, analogs etc) in the polypeptide can be distinguished by their physical size and charge. The amino acids/derivatives/analogs can be naturally occurring or artificial. The polypeptide may comprise any naturally occurring amino acid.

The polypeptide may be modified. The polypeptide may be modified for detection using the method of the invention. The method may be for characterising modifications in the target polypeptide.

One or more of the amino acids/derivatives/analogs in the polypeptide may be modified. One or more of the amino acids/derivatives/analogs in the polypeptide may be post-translationally modified. As such, the method of the invention can be used to detect the presence, absence, number of positions of post-translational modifications in a polypeptide. The method can be used to characterise the extent to which a polypeptide has been post-translationally modified.

Any one or more post-translational modifications may be present in the polypeptide. Typical post-translational modifications include modification with a hydrophobic group, modification with a cofactor, addition of a chemical group, glycation (the non-enzymatic attachment of a sugar), phosphorylation, biotinylation and pegylation. Post-translational modifications can also be non-natural, such that they are chemical modifications done in the laboratory for biotechnological or biomedical purposes. This can allow monitoring the levels of the laboratory made peptide, polypeptide, or protein in contrast to the natural counterparts.

Examples of post-translational modification with a hydrophobic group include myristoylation, attachment of myristate, a C₁₄ saturated acid; palmitoylation, attachment of palmitate, a C₁₆ saturated acid; isoprenylation or prenylation, the attachment of an isoprenoid group; farnesylation, the attachment of a farnesol group; geranylgeranylation, the attachment of a geranylgeraniol group; and glypiation, and glycosylphosphatidylinositol (GPI) anchor formation via an amide bond.

Examples of post-translational modification with a cofactor include lipoylation, attachment of a lipoate (C₈) functional group; flavination, attachment of a flavin moiety (e.g. flavin mononucleotide (FMN) or flavin adenine dinucleotide (FAD)); attachment of heme C, for instance via a thioether bond with cysteine; phosphopantetheinylation, the attachment of a 4′-phosphopantetheinyl group; and retinylidene Schiff base formation.

Examples of post-translational modification by addition of a chemical group include acylation, e.g. O-acylation (esters), N-acylation (amides) or S-acylation (thioesters); acetylation, the attachment of an acetyl group for instance to the N-terminus or to lysine; formylation; alkylation, the addition of an alkyl group, such as methyl or ethyl; methylation, the addition of a methyl group for instance to lysine or arginine; amidation; butyrylation; gamma-carboxylation; glycosylation, the enzymatic attachment of a glycosyl group for instance to arginine, asparagine, cysteine, hydroxylysine, serine, threonine, tyrosine or tryptophan; polysialylation, the attachment of polysialic acid; malonylation; hydroxylation; iodination; bromination; citrulination; nucleotide addition, the attachment of any nucleotide such as any of those discussed above, ADP ribosylation; oxidation; phosphorylation, the attachment of a phosphate group for instance to serine, threonine or tyrosine (O-linked) or histidine (N-linked); adenylylation, the attachment of an adenylyl moiety for instance to tyrosine (O-linked) or to histidine or lysine (N-linked); propionylation; pyroglutamate formation; S-glutathionylation; Sumoylation; S-nitrosylation; succinylation, the attachment of a succinyl group for instance to lysine; selenoylation, the incorporation of selenium; and ubiquitinilation, the addition of ubiquitin subunits (N-linked).

The polypeptide may be labelled with a molecular label. A molecular label may be a modification to the polypeptide which promotes the detection of the polypeptide in the method of the invention. For example, the label may be a modification to the polypeptide which alters the signal obtained as conjugate is characterised. For example, the label may interfere with a flux of ions through the nanopore. In such a manner, the label may improve the sensitivity of the method.

The polypeptide may contain one or more cross-linked sections, e.g., C-C bridges. The polypeptide may not be cross-linked prior to being characterised using the method.

The polypeptide may comprise sulphide-containing amino acids and thus has the potential to form disulphide bonds. Typically, in such embodiments, the polypeptide is reduced using a reagent such as DTT (Dithiothreitol) or TCEP (tris(2-carboxyethyl)phosphine) prior to being characterised using the method.

The polypeptide may be a full-length protein or naturally occurring polypeptide. The protein or naturally occurring polypeptide may be fragmented prior to conjugation to the polynucleotide. The polypeptide may be chemically or enzymatically fragmented. The polypeptides or polypeptide fragments can be conjugated to form a longer target polypeptide.

The polypeptide can be any suitable length. The polypeptide preferably has a length of from about 2 to about 300 peptide units or amino acids. The polypeptide has a length of from about 2 to about 100 peptide units, for example from about 2 to about 50 peptide units, e.g., from about 3 to about 50 peptide units, such as from about 5 to about 25 peptide units, e.g., from about 7 to about 16 peptide units, such as from about 9 to about 12 peptide units. “Peptide unit” is interchangeable with “amino acid”.

The one or more characteristics of the polypeptide are preferably selected from (i) the length of the polypeptide, (ii) the identity of the polypeptide, (iii) the sequence of the polypeptide, (iv) the secondary structure of the polypeptide and (v) whether or not the polypeptide is modified. The one or more characteristics may be the sequence of the polypeptide or whether or not the polypeptide is modified, e.g., by one or more post-translational modifications. The one or more characteristics are preferably the sequence of the polypeptide.

The polypeptide may be in a relaxed form. The polypeptide may be held in a linearized form. Holding the polypeptide in a linearized form can facilitate the characterisation of the polypeptide on a residue-by-residue basis as “bunching up” of the polypeptide within the nanopore is prevented. The polypeptide can be held in a linearized form using any suitable means. For example, if the polypeptide is charged, the polypeptide can be held in a linearized form by applying a voltage.

If the polypeptide is not charged or is only weakly charged then the charge can be altered or controlled by adjusting the pH. For example, the polypeptide can be held in a linearized form by using high pH to increase the relative negative charge of the polypeptide. Increasing the negative charge of the polypeptide allows it to be held in a linearized form under, e.g., a positive voltage. Alternatively, the polypeptide can be held in a linearized form by using low pH to increase the relative positive charge of the polypeptide. Increasing the positive charge of the polypeptide allows it to be held in a linearized form under, e.g., a negative voltage. In the disclosed methods a polynucleotide-handling protein is used to control the movement of a polynucleotide with respect to a nanopore. As a polynucleotide is typically negatively charged it is generally most suitable to increase the linearization of the polypeptide by increasing the pH thus making the polypeptide more negatively charged, in common with the polynucleotide. In this way, the conjugate retains an overall negative charge and thus can readily move, e.g., under an applied voltage.

The polypeptide can be held in a linearized form by using suitable denaturing conditions. Suitable denaturing conditions include, for example, the presence of appropriate concentrations of denaturants such as guanidine HCl and/or urea. The concentration of such denaturants to use in the disclosed methods is dependent on the target polypeptide to be characterised in the methods and can be readily selected by those of skill in the art.

The polypeptide can be held in a linearized form by using suitable detergents. Suitable detergents for use in the disclosed methods include SDS (sodium dodecyl sulfate). The polypeptide can be held in a linearized form by carrying out the disclosed methods at an elevated temperature. Increasing the temperature overcomes intra-strand bonding and allows the polypeptide to adopt a linearized form.

The polypeptide can be held in a linearized form by carrying out the method under strong electro-osmotic forces. Such forces can be provided by using asymmetric salt conditions and/or providing suitable charge in the channel of the nanopore. The charge in the channel of a pore can be altered, e.g., by mutagenesis. Altering the charge of a pore is well within the capacity of those skilled in the art. Altering the charge of a pore generates strong electro-osmotic forces from the unbalanced flow of cations and anions through the nanopore when a voltage potential is applied across the nanopore.

The polypeptide can be held in a linearized form by passing it through a structure such an array of nanopillars, through a nanoslit or across a nanogap. The physical constraints of such structures can force the polypeptide to adopt a linearized form.

The target polypeptide may be cleaved or fragmented to form smaller target peptides before it is contacted with the actinoporin pore of the invention, pore multimer of the invention or membrane of the invention. Suitable methods for doing this are known in the art, for instance using proteolytic enzymes such as trypsin, chymotrypsin, or Glu-C. This type of approach is known as protein fingerprinting. The method may comprise cleaving or fragmenting the target polypeptide to form smaller target peptides, contacting the target peptides with an actinoporin pore of the invention, a pore multimer of the invention or a membrane of the invention such that the target peptides moves with respect to, such as into or through, the actinoporin pore, pore multimer or membrane and taking one or more measurements as the target peptides moves with respect to the actinoporin pore or pore multimer and thereby determining the presence, absence or one or more characteristics of the target polypeptide.

The target polypeptide is preferably positively charged. Suitable proteins include, but are not limited to, those shown in the table below.

| | Analyte | UniProt | organism | Net charge*** | aa/MW |
|---|---|---|---|---|---|
| 1* | H3.1 | P68431 | human | 18.4 | 136/15.404 |
| 2* | H4 | P62805 | human | 16.9 | 103/11.367 |
| 3* | H3Cit (8x) | | human | 9.4 | |
| 4* | IGF1 hormone | P05019 | human | 15.0 | 15/21.841 |
| 5* | Neuropeptide-like protein 7 | Peptide | | 2.6 | 99/11.780 |
| | | hormone | | | |
| 6* | Endothelin-2 | Peptide | | 9.3 | 79/8.890 |
| | | hormone | | | |
| 7* | Nucleoprotein (SARS CoV)) | P0DTC9 | virus | 22.5 | 419/45.626 |
| 8* | Membrane protein (SARS CoV) | P59596 | virus | 5.9 | 221/25.061 |
| 9* | Interferon gamma precursor | P01579 | human | 7.6 | 166/19.348 |
| 10* | Protamine-2 | P04554 | human | 23.1 | 102/13.051 |
| 11 | Bacterial histones | Q6MRM1 | bacteria | 0.6 | 64/6.982 |
| 12** | Mitochondrial-derived | A0A0C5B5G6 | human | 2.0 | 16/2.174 |
| | peptide MOTS-c | | | | |
| 13** | AMP-responsive element- | O60519 | human | 10.8 | 120/13.784 |
| | binding protein-like 2 | | | | |
| 14** | Musculin | O60682 | human | 3.5 | 206/8.886 |
| 15** | Heart- and neural crest | O96004 | human | 7.3 | 215/23.612 |
| | derivatives-expressed | | | | |
| | protein 1 | | | | |
| 16** | Non-histone chromosomal | P05204 | human | 10.8 | 90/9.393 |
| | protein HMG-17 | | | | |
| 17** | High mobility group protein | P17096 | human | 10.8 | 107/11.676 |
| | HMG-I/HMG-Y | | | | |
| 18** | CCAAT/enhancer-binding | P53567 | human | 5.89 | 150/16.408 |
| | protein gamma | | | | |

*Search in Biomarker database https://markerdb.ca/proteins/171
**UniProt: DNA binding proteins up to 250 amino acids long
**Net charges at pH 8 calculated with: https://www.protpi.ch/Calculator/

The positively charged polypeptide is preferably a histone. The histone is preferably selected from H3.1, H4, H3Cit, H3K9ac and H3K23ac In addition to their physiological canonical function in DNA packaging and regulation of gene expression in the cell nucleus, histones also have extracellular functions. They have antimicrobial functions and can act as damage-associated molecular patterns (DAMPs; see reference 15 in Example 2) and are a predominant protein in neutrophil extracellular traps (NETs) (see references 15 and 16 in Example 2).

The invention also provides a method of discriminating two or more target analytes, preferably two or more target polypeptides, in a mixture of analytes. The method involves contacting the mixture of analytes with an actinoporin pore of the invention, a pore multimer of the invention or a membrane of the invention such that the two or more target analytes move with respect to, such as into or through, the actinoporin pore, pore multimer or membrane and taking one or more measurements as the two or more target analytes move with respect to the actinoporin pore or pore multimer and thereby discriminating the two or more target analytes in the mixture. The two or more analytes are preferably two or more histones. The two or more histones are preferably selected from H3.1, H4, H3Cit, H3K9ac and H3K23ac. The two or more histones preferably comprise or consist of H4 and H3Cit.

The target analyte is preferably a polynucleotide, such as a nucleic acid, which is defined as a macromolecule comprising two or more nucleotides. Nucleic acids are particularly suitable for nanopore sequencing. The naturally occurring nucleic acid bases in DNA and RNA may be distinguished by their physical size. As a nucleic acid molecule, or individual base, passes through the channel of a nanopore, the size differential between the bases causes a directly correlated reduction in the ion flow through the channel. The variation in ion flow may be recorded. Suitable electrical measurement techniques for recording ion flow variations are discussed above. Through suitable calibration, the characteristic reduction in ion flow can be used to identify the particular nucleotide and associated base traversing the channel in real-time. In typical nanopore nucleic acid sequencing, the open-channel ion flow is reduced as the individual nucleotides of the nucleic sequence of interest sequentially pass through the channel of the nanopore due to the partial blockage of the channel by the nucleotide. It is this reduction in ion flow that is measured using the suitable recording techniques described above. The reduction in ion flow may be calibrated to the reduction in measured ion flow for known nucleotides through the channel resulting in a means for determining which nucleotide is passing through the channel, and therefore, when done sequentially, a way of determining the nucleotide sequence of the nucleic acid passing through the nanopore. For the accurate determination of individual nucleotides, it has typically required for the reduction in ion flow through the channel to be directly correlated to the size of the individual nucleotide passing through the constriction. It will be appreciated that sequencing may be performed upon an intact nucleic acid polymer that is ‘threaded’ through the pore via the action of an associated polymerase, for example. Alternatively, sequences may be determined by passage of nucleotide triphosphate bases that have been sequentially removed from a target nucleic acid in proximity to the pore (see for example WO 2014/187924 incorporated herein by reference in its entirety).

The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag, for which suitable examples are known by a skilled person. The polynucleotide may comprise one or more spacers. A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate, or triphosphate. The nucleotide may comprise more than three phosphates, such as 4 or 5 phosphates. Phosphates may be attached on the 5′ or 3′ side of a nucleotide. The nucleotides in the polynucleotide may be attached to each other in any manner. The nucleotides are typically attached by their sugar and phosphate groups as in nucleic acids. The nucleotides may be connected via their nucleobases as in pyrimidine dimers. The polynucleotide may be single stranded or double stranded. At least a portion of the polynucleotide is preferably double stranded. The polynucleotide is most preferably ribonucleic nucleic acid (RNA) or deoxyribonucleic acid (DNA). In particular, said method using a polynucleotide as an analyte alternatively comprises determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

The polynucleotide can be any length (i). For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. Any number of polynucleotides can be investigated. For instance, the method may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide. The polynucleotide can be naturally occurring or artificial. For instance, the method may be used to verify the sequence of a manufactured oligonucleotide. The method is typically carried out in vitro.

Nucleotides can have any identity (ii), and include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP. A nucleotide may be abasic (i.e., lack a nucleobase). A nucleotide may also lack a nucleobase and a sugar (i.e., is a C3 spacer). The sequence of the nucleotides (iii) is determined by the consecutive identity of following nucleotides attached to each other throughout the polynucleotide strain, in the 5′ to 3′ direction of the strand.

In particular, said method using a polynucleotide as an analyte alternatively comprises determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

The polynucleotide can be any length (i). For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. Any number of polynucleotides can be investigated. For instance, the method may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide. The polynucleotide can be naturally occurring or artificial. For instance, the method may be used to verify the sequence of a manufactured oligonucleotide. The method is typically carried out in vitro.

Nucleotides can have any identity (ii). Possible nucleotides are defined above with reference to the actinoporin monomers of the invention. The sequence of the nucleotides (iii) is determined by the consecutive identity of following nucleotides attached to each other throughout the polynucleotide strain, in the 5′ to 3′ direction of the strand.

The actinoporin pores and pore multimers of the invention are particularly useful in analysing homopolymers. For example, they may be used to determine the sequence of a polynucleotide comprising two or more, such as at least 3, 4, 5, 6, 7, 8, 9 or 10, consecutive nucleotides that are identical. For example, they may be used to sequence a polynucleotide comprising a polyA, polyT, polyG and/or polyC region.

The target analyte may comprise a polynucleotide and a polypeptide. The target analyte may be a polynucleotide-polypeptide conjugate. The conjugate preferably comprises a polynucleotide conjugated to a polypeptide. One or both of the polynucleotide and polypeptide may be the target and may be characterised in accordance with the invention.

The polypeptide can be conjugate to the polynucleotide at any suitable position. For example, the polypeptide can be conjugated to the polynucleotide at the N-terminus or the C-terminus of the polypeptide. The polypeptide can be conjugated to the polynucleotide via a side chain group of a residue (e.g., an amino acid residue) in the polypeptide. The polypeptide may have a naturally occurring reactive functional group which can be used to facilitate conjugation to the polynucleotide. For example, a cysteine residue can be used to form a disulphide bond to the polynucleotide or to a modified group thereon.

The polypeptide may be modified in order to facilitate its conjugation to the polynucleotide. For example, the polypeptide may be modified by attaching a moiety comprising a reactive functional group for attaching to the polynucleotide. For example, the polypeptide can be extended at the N-terminus or the C-terminus by one or more residues (e.g., amino acid residues) comprising one or more reactive functional groups for reacting with a corresponding reactive functional group on the polynucleotide. For example, the polypeptide can be extended at the N-terminus and/or the C-terminus by one or more cysteine residues. Such residues can be used for attachment to the polynucleotide portion of the conjugate, e.g., by maleimide chemistry (e.g., by reaction of cysteine with an azido-maleimide compound such as azido-[Pol]-maleimide wherein [Pol] is typically a short chain polymer such as PEG, e.g., PEG2, PEG3, or PEG4; followed by coupling to appropriately functionalised polynucleotide e.g., polynucleotide carrying a BCN group for reaction with the azide). Such chemistry is described in Example 2. For avoidance of doubt, when the polypeptide comprises an appropriate naturally occurring residue at the N- and/or C-terminus (e.g., a naturally occurring cysteine residue at the N- and/or C-terminus) then such residue(s) can be used for attachment to the polynucleotide.

A residue in the polypeptide may be modified to facilitate attachment of the polypeptide to the polynucleotide. A residue (e.g., an amino acid residue) in the polypeptide may be chemically modified for attachment to the polynucleotide. A residue (e.g., an amino acid residue) in the polypeptide may be enzymatically modified for attachment to the polynucleotide.

The conjugation chemistry between the polynucleotide and the polypeptide in the conjugate is not particularly limited. Any suitable combination of reactive functional groups can be used. Many suitable reactive groups and their chemical targets are known in the art. Some exemplary reactive groups and their corresponding targets include aryl azides which may react with amine, carbodiimides which may react with amines and carboxyl groups, hydrazides which may react with carbohydrates, hydroxymethyl phosphines which may react with amines, imidoesters which may react with amines, isocyanates which may react with hydroxyl groups, carbonyls which may react with hydrazines, maleimides which may react with sulfhydryl groups, NHS-esters which may react with amines, PFP-esters which may react with amines, psoralens which may react with thymine, pyridyl disulfides which may react with sulfhydryl groups, vinyl sulfones which may react with sulfhydryl amines and hydroxyl groups, vinylsulfonamides, and the like. Other suitable chemistry for conjugating the polypeptide to the polynucleotide includes click chemistry. Many suitable click chemistry reagents are known in the art. Suitable examples of click chemistry include, but are not limited to, the following:
copper(I)-catalyzed azide-alkyne cycloadditions (azide alkyne Huisgen cycloadditions);
strain-promoted azide-alkyne cycloadditions; including alkene and azide [3+2]cycloadditions; alkene and tetrazine inverse-demand Diels-Alder reactions; and alkene and tetrazole photoclick reactions;
copper-free variant of the 1,3 dipolar cycloaddition reaction, where an azide reacts with an alkyne under strain, for example in a cyclooctane ring such as in bicycle[6.1.0]nonyne (BCN);
the reaction of an oxygen nucleophile on one linker with an epoxide or aziridine reactive moiety on the other; and
the Staudinger ligation, where the alkyne moiety can be replaced by an aryl phosphine, resulting in a specific reaction with the azide to give an amide bond.

Any reactive group may be used to form the conjugate. Some suitable reactive groups include [1, 4-Bis[3-(2-pyridyldithio)propionamido]butane; 1,1 1-bis-maleimidotriethyleneglycol; 3,3′-dithiodipropionic acid di(N-hydroxysuccinimide ester); ethylene glycol-bis(succinic acid N-hydroxysuccinimide ester); 4,4′-diisothiocyanatostilbene-2,2′-disulfonic acid disodium salt; Bis[2-(4-azidosalicylamido)ethyl]disulphide; 3-(2-pyridyldithio)propionic acid N-hydroxysuccinimide ester; 4-maleimidobutyric acid N-hydroxysuccinimide ester; Iodoacetic acid N-hydroxysuccinimide ester; S-acetylthioglycolic acid N-hydroxysuccinimide ester; azide-PEG-maleimide; and alkyne-PEG-maleimide. The reactive group may be any of those disclosed in WO 2010/086602, particularly in Table 3 of that application.

The reactive functional group may be comprised in the polynucleotide and the target functional group may be comprised in the polypeptide prior to the conjugation step. The reactive functional group may be comprised in the polypeptide and the target functional group may be comprised in the polynucleotide prior to the conjugation step. The reactive functional group may be attached directly to the polypeptide. The reactive functional group may be attached to the polypeptide via a spacer. Any suitable spacer can be used. Suitable spacers include for example alkyl diamines such as ethyl diamine, etc.

The conjugate may comprise a plurality of polypeptide sections and/or a plurality of polynucleotide sections. For example, the conjugate may comprise a structure of the form . . . -P-N-P-N-P-N . . . wherein P is a polypeptide and N is a polynucleotide. A polynucleotide-handling protein may sequentially control the N portions of the conjugate with respect to the pore and thus sequentially controls the movement of the P sections with respect to the pore, thus allowing the sequential characterisation of the P sections. The plurality of polynucleotides and polypeptides may be conjugated together by the same or different chemistries.

The conjugate may comprise a leader. Any suitable leader may be used. The leader may be a polynucleotide. The leader may be the same sort of polynucleotide as the polynucleotide used in the conjugate, or it may be a different type of polynucleotide. For example, the polynucleotide in the conjugate may be DNA and the leader may be RNA or vice versa.

The leader may be a charged polymer, e.g., a negatively charged polymer. The leader may comprise a polymer such as PEG or a polysaccharide. The leader may be from 10 to 150 monomer units (e.g., ethylene glycol or saccharide units) in length, such as from 20 to 120, e.g., 30 to 100, for example 40 to 80 such as 50 to 70 monomer units (e.g., ethylene glycol or saccharide units) in length. The methods of characterising a target polypeptide of the invention may comprise conjugating a polypeptide to a polynucleotide.

The movement of the analyte with respect to the pore, such as through the pore, is preferably controlled using an analyte binding protein, such as a polynucleotide binding protein. Suitable proteins are discussed in more detail above. The invention provides a method for determining the presence, absence or one or more characteristics of a target polynucleotide, comprising the steps of:
(i) contacting the target polynucleotide with an actinoporin pore of the invention, a pore multimer of the invention or a membrane of the invention and a polynucleotide binding protein, such that the polynucleotide binding protein controls the movement of the target polynucleotide with respect to, such as through, the actinoporin pore, the pore multimer or the membrane; and
(ii) taking one or more measurements as the polynucleotide moves with respect to, such as through, the actinoporin pore, the pore multimer or the membrane and thereby determining the presence, absence or one or more characteristics of the polynucleotide.

The method for determining the presence, absence or one or more characteristics of a target polynucleotide may involve the use of one or more sequencing adaptors. The skilled person is capable of using sequencing adaptors, such as the adaptors described in WO 2016/034591 and WO 2018/100370 (both incorporated herein by reference in their entirety), to attach a suitable portion or region to a double stranded polynucleotide. These adaptors also comprise suitable binding sites for polynucleotide binding proteins. The skilled person is also capable of designing a functional binding moiety comprising a portion or region that is capable of hybridising to the revealed portion or region.

In any of the methods, the one or more characteristics of the target analyte are preferably measured by electrical measurement and/or optical measurement. The electrical measurement is a current measurement, an impedance measurement, a tunnelling measurement, or a field effect transistor (FET) measurement. The method preferably comprises measuring the current flowing through the actinoporin pore or the pore multimer as the target analyte moves with respect to, such as through, the pore.

General conditions for conducting the methods of the invention are discussed in more detail below with reference to the kits and systems of the invention.

### Polynucleotides of the Invention

The invention also provides a polynucleotide which encodes an actinoporin pore of the invention. The polynucleotide may be any of those discussed above. The invention also provides an expression vector comprising a polynucleotide of the invention. The invention also provides a host cell comprising a polynucleotide of the invention or a vector of the invention. Suitable vectors and host cells are known in the art.

### Kits

The invention also provides kits for characterising a target analyte. The kit comprises (a) an actinoporin monomer of the invention, a construct of the invention, an actinoporin pore of the invention or a pore multimer of the invention and (b) the components of a membrane and/or an analyte binding protein. The kit preferably comprises (a) an actinoporin monomer of the invention, a construct of the invention, an actinoporin pore of the invention or a pore multimer of the invention and (b) the components of a membrane. The kit preferably comprises (a) an actinoporin monomer of the invention, a construct of the invention, an actinoporin pore of the invention or a pore multimer of the invention and (b) an analyte binding protein. The kit preferably comprises (a) an actinoporin monomer of the invention, a construct of the invention, an actinoporin pore of the invention or a pore multimer of the invention and (b) the components of a membrane and an analyte binding protein.

The kit may comprise components of any type of membranes, such as an amphiphilic layer, such as a triblock copolymer membrane. The membrane is preferably artificial. The analyte binding protein is preferably a polynucleotide binding protein. Suitable proteins are described above.

The kit may further comprise one or more anchors, such as cholesterol, for coupling the target analyte to the membrane. The kit may further comprise one or more polynucleotide adaptors, such as one or more sequencing adaptors, that can be attached to a target polynucleotide to facilitate characterisation of the polynucleotide. The anchor, such as cholesterol, is preferably attached to the polynucleotide adaptor.

The kit may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides or voltage or patch clamp apparatus. Reagents may be present in the kit in a dry state such that a fluid sample resuspends the reagents. The kit may also, optionally, comprise instructions to enable the kit to be used in the method of the invention or details regarding for which organism the method may be used. Finally, the kit may also comprise additional components useful in analyte characterization.

### Apparatus

The invention also provides an apparatus for characterising target analytes in a sample, comprising (a) a plurality of actinoporin monomers of the invention, a plurality of constructs of the invention, a plurality of actinoporin pores of the invention or a plurality of pore multimers of the invention and (b) a plurality of analyte binding proteins, preferably polynucleotide binding proteins. The plurality of actinoporin monomers, constructs, actinoporin pores or pore multimers may be any of those discussed above. Suitable polynucleotide binding proteins are also discussed above.

The invention also provides an apparatus comprising an actinoporin pore derived from Orbicella faveolata, such as an actinoporin pore of the invention or a pore multimer of the invention, inserted into an in vitro membrane.

The invention also provides an apparatus produced by a method comprising: (i) obtaining an actinoporin pore derived from Orbicella faveolata, such as an actinoporin pore of the invention or a pore multimer of the invention, and (ii) contacting the actinoporin pore derived from Orbicella faveolata, such as the actinoporin pore of the invention or pore multimer of the invention, with an in vitro membrane such that the actinoporin pore or pore multimer is inserted in the in vitro membrane.

Any of the specific embodiments discussed above are equally applicable to the apparatuses of the invention.

### Arrays

The invention also provides an array comprising a plurality of membranes of the invention. Any of the embodiments discussed above with respect to the membranes of the invention equally apply the array of the invention. The array may be set up to perform any of the methods described below.

In one embodiment, each membrane in the array comprises one actinoporin pore or pore multimer. Due to the manner in which the array is formed, for example, the array may comprise one or more membranes that do not comprise an actinoporin pore or pore multimer, and/or one or more membranes that comprise two or more pores complexes or multimers. The array may comprise from about 2 to about 1000, such as from about 10 to about 800, from about 20 to about 600 or from about 30 to about 500 membranes.

### System

The invention provides a system comprising (a) a membrane of the invention or an array of the invention, (b) means for applying a potential across the membrane(s) and (c) means for detecting electrical or optical signals across the membrane(s). The electrical signal may be a measurement of ion flow through the nanopore such as the measurement of a current or voltage over time.

The actinoporin pores and membranes may be any as described above and below.

In one embodiment, the system further comprises a first chamber and a second chamber, wherein the first and second chambers are separated by the membrane(s). When used to characterise a target analyte, the system may further comprise a target analyte, wherein the target analyte is transiently located within the continuous channel and wherein one end of the target analyte is located in the first chamber and one end of the target analyte is located in the second chamber. The target analyte is preferably a target polypeptide or a target polynucleotide.

In one embodiment, the system further comprises an electrically conductive solution in contact with the pore(s), electrodes providing a voltage potential across the membrane(s), and a measurement system for measuring the current through the pore(s). The voltage applied across the membranes and pore is preferably from +5 V to −5 V, such as −600 mV to +600 mV or −400 mV to +400 mV. The voltage used is preferably in the range 100 mV to 240 mV and more preferably in the range of 120 mV to 220 mV. It is possible to increase discrimination between different amino acids or nucleotides by a pore by using an increased applied potential. Any suitable electrically conductive solution may be used. For example, the solution may comprise charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In an exemplary system, salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane, e.g., in each chamber.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of an amino acid or nucleotide to be identified against the background of normal current fluctuations.

A buffer may be present in the electrically conductive solution. Typically, the buffer is phosphate buffer. Other suitable buffers are HEPES and Tris-HCl buffer. The pH of the electrically conductive solution may be from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The system may be comprised in an apparatus. The apparatus may be any conventional apparatus for analyte analysis, such as an array or a chip. The apparatus is preferably set up to carry out the disclosed method. For example, the apparatus may comprise a chamber comprising an aqueous solution and a barrier that separates the chamber into two sections. The barrier typically has an aperture in which the membrane(s) containing the pore(s) are formed. Alternatively, the barrier forms the membrane in which the pore is present.

The apparatus may also comprise an electrical circuit capable of applying a potential and measuring an electrical signal across the membrane and pore.

The apparatus may be any of those described in WO 2008/102120, WO 2009/077734, WO 2010/122293, WO 2011/067559, WO 2014/06442, or WO2020/183172 (all incorporated herein by reference in their entirety).

### Analysis of Measurements

The method for determining the presence, absence or one or more characteristics of a target polymer analyte may comprise estimating or determining the sequence of polymer units. The signal measured during movement of the polymer, such as a polypeptide, polynucleotide, or polypeptide-polynucleotide conjugate, with respect to the actinoporin pore or pore multimer may be dependent at any one time upon multiple polymer units such as amino acids or nucleotides. For example, the presence of multiple amino acids or nucleotides in the lumen of the actinoporin pore or pore multimer and potentially amino acids or nucleotides outside of the actinoporin pore or pore multimer can influence the ion flow and therefore current or voltage signal. The polypeptide or polynucleotide may also contain modified amino acids or nucleotides which can affect the measurement signal and as such the estimation or determination of the sequence may be non-trivial. Various known mathematical techniques and variations thereof may be used to determine or estimate the polymer sequence, including probabilistic and machine learning techniques. Such methods are described for example in WO2013041878, WO2013121224, WO2018203084 and Zhang et al: A Guide to Signal Processing Algorithms for Nanopore Sensors, ACS Sens. 2021, 6, 10, 3536-3555, all of which are hereby incorporated by reference in their entirety.

The method of the invention may comprise the measurement of target analyte wherein measurements can be used to estimate or determine an overall sequence. Various known methods may be used. For example, the sequence may be initially determined from the series of measurements taken during the movement of the analyte with respect to the actinoporin pore or pore multimer and the results combined to provide an overall sequence. More preferably the series of measurements may be treated by a probabilistic or machine learning technique as plural series of measurements in plural dimensions wherein an overall sequence determination is made without the initial determination of the sequence of the analyte. A non-limiting example of a method suitable for use in the invention is disclosed in WO2015140535 (incorporated by reference in its entirety).

### Membrane

Any suitable membrane may be used in the system. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e., lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units) but may also be constructed from more than two monomer sub-units to form more complex arrangements that behave as amphiphiles. The amphiphilic layer may comprise a diblock, triblock, tetrablock or pentablock copolymer.

The membrane may comprise one of the membranes disclosed in International Application No. WO 2014/064443 or WO 2014/064444 (both incorporated herein by reference in their entirety).

The amphiphilic molecules may be chemically modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two-dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer, or a liposome. The lipid bilayer is preferably a planar lipid bilayer. Suitable lipid bilayers are disclosed in WO 2008/102121, WO 2009/077734, and WO 2006/100484 (all incorporated herein by reference in their entirety).

The membrane may comprise a solid-state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid-state layer may be formed from graphene. Suitable graphene layers are disclosed in WO 2009/035647 (incorporated herein by reference in its entirety). If the membrane comprises a solid-state layer, the pore is typically present in an amphiphilic membrane or layer contained within the solid-state layer, for instance within a hole, well, gap, channel, trench or slit within the solid-state layer. The skilled person can prepare suitable solid state/amphiphilic hybrid systems. Suitable systems are disclosed in WO 2009/020682 and WO 2012/005857 (both incorporated herein by reference in their entirety). Any of the amphiphilic membranes or layers discussed above may be used.

Preferred embodiments include (i) an artificial amphiphilic layer comprising an actinoporin pore or pore multimer, (ii) an isolated, naturally occurring lipid bilayer comprising an actinoporin pore or pore multimer, or (iii) a cell having an actinoporin pore or pore multimer inserted therein. The most preferred embodiment is an artificial amphiphilic layer, such as a di- or tri-block copolymer layer. The layer may comprise other transmembrane and/or intramembrane proteins as well as other molecules in addition to the actinoporin pore or pore multimer. Suitable apparatus and conditions are discussed below. All embodiments of the invention are typically carried out in vitro.

SEQUENCE LISTING
SEQ ID NO: 1
SAPIKANDPNGEVLEEMPKTKRGAEALLADVGLAHFPEMVPNRQE
LRALLKRSHNAEAIPQEPMDLENLDSEKRAARIAAGTIIAGAELT
IGLLQNLLDVLANVNRKCAVGVDNESGFRWQEGSTYFFSGTADEN
LPYSVSDGYAVLYGPRKTNGPVATGVVGVLAYYIPSIGKTLAVMW
SVPFDYNFYQNWWNAKLYSGNQDADYDHYVDLYYDANPFKANGWH
ERSLGSGLKFCGSMSSSGQATLEIHVLKESETCM
SEQ ID NO: 2
RIAAGTIIAGAELTIGLLQNLLDVLANVNRKCAVGVDNESGFRWQ
EGSTYFFSGTADENLPYSVSDGYAVLYGPRKTNGPVATGVVGVLA
YYIPSIGKTLAVMWSVPFDYNFYQNWWNAKLYSGNQDADYDHYVD
LYYDANPFKANGWHERSLGSGLKFCGSMSSSGQATLEIHVLKESE
TCM

The following Examples illustrate the invention. It is to be understood that although particular embodiments, specific configurations as well as materials and/or molecules, have been discussed herein for engineered cells and methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1

### Protein Expression and Purification

The nucleotide sequence for Fav constructs was inserted into a modified pEt28a (+) plasmid with N-terminal 6×His following TEV restriction site (ENLYFQS). 100 μl of competent cells E. coli BL21 strain transformed with 1.5 μl plasmid (heat shock) was applied to agar plates with kanamycin resistance and incubated overnight at 37° C. A single colony was inoculated into 10 mL of LB medium (30 μg/ml kanamycin) and incubated overnight at 37° C. and 180 rotations/min. The overnight culture was then added to 1 L of TB medium (30 μg/ml kanamycin) and incubated at 37° C. till reaching A₆₀₀ 0.8 when protein expression was induced by 0.25 mM IPTG. Cells were then grown at 18° C. and were harvested after 16-18 h by centrifuging biomass at 4000×g, for 10 min and 4° C. E. coli BL21 cells expressing Fav constructs were resuspended in PBS buffer with 10 mM imidazole, sonicated by ultrasound sonication and affinity-purified by using nickel affinity chromatography. TEV cleavage, for N-terminal tag removal was done during overnight dialysis against PBS buffer.

### Lipid Vesicles

50 mM multilamellar vesicles composed of SM (brain, Porcine) and DOPC in a molar ratio 1:1 were prepared by prepared by solubilising lipids supplied from Avanti Polar Lipids (Alabaster, AL, USA) in chloroform, evaporating the solvents by rotavaporation (lipid film formation) and addition of PBS. After freeze thaw cycles formed MLVs were extruded over 100 nm membrane to form 100 mM large unilamellar vesicles used in pore formation assay.

### Pores

Pores were prepared by incubating soluble Fav monomers (1h, 37° C.) with 50 mM large unilamellarvesicles (molar ration 215:1). Vesicles were disrupted with LDAO, and soluble pores purified by ion exchange chromatograph (resource Q, Cytiva, 50 mM Tris/HCl, 0.02% Brij 35, pH 8). The pore formation was checked by Native PAGE and Cryo-EM. Samples for Native PAGE were prepared by mixing 17 μL of pore samples (after ion exchange chromatography), 6 μL of native PAGE loading buffer (50 mM Bis/Tris, 6N HCl, 50 mM NaCl, 10% w/v Glycerol 0,001% Ponceau S, pH=7.2) and G-250 (final concentration 0.02%), centrifuged for 10 min at 16 000×g and loaded on 4-16% Bis/Tris gels.

The sequence of one monomer from wild type Fav (wtFav) is shown in SEQ ID NO: 1. Deletion mutants are indicated using AXX where XX is the number of amino acids deleted (e.g., Δ53 explains 53 amino acids are deleted). N-terminal deletions have no letter between the A and number, but C-terminal deletions include a C between the A and number (e.g., ΔC5). Substitutions are shown using the standard nomenclature and the numbering of positions in SEQ ID NO: 1 (e.g., D203R relates to substitution of D at position 203 of SEQ ID NO: 1 with R). RN-Fav is SEQ ID NO: 1 with ΔN76, D203R, and D215N. RN1-Fav is SEQ ID NO: 1 with ΔN67, E67S, N68E, K73N, R74D, R77D, I91F, D99Y, V100F, L101F, D203R, and D215N.

### Electrophysical Measurements

Pore insertions and analyte detection was done with MinION set up and C18 buffer. Soluble Fav pore were diluted in C18 buffer and inserted via standard pore insertion script. For histone detection at +50 mV, histone samples were diluted in 90 μL of C18 buffer and loaded on MinION flow cell.

### Cryo-EM

Cryo-EM grids for all Fav pores were prepared the same way. 3 μL of Fav pore sample was applied to a glow-discharged (GloQube® Plus, Quorum, UK) Quantifoil R1.2/1.3 200-mesh copper holey carbon grid (Quantifoil, Germany), blotted under 95% humidity at 4° C. for 6.5-7 s and plunged into liquid ethane using a Mark IV Vitrobot (Thermo Fisher Scientific, USA). Micrographs were collected on a cryo-transmission electron microscope (Glacios, Thermo Fisher Scientific, USA) with a Falcon 3EC direct electron detector (Thermo Fisher Scientific, USA) and operated at 200 kV using the EPU software (Thermo Fisher Scientific, USA). Images were recorded in counting mode with a pixel size of 0.47 Å. Micrographs were dose-fractioned into 30 frames with a total dose of 30 e−/Å.

Data processing was performed with cryoSPARC. Micrographs were aligned and summed using Patch motion correction. Contrast transfer function (CTF) estimation was performed using Patch CTF. Micrographs with CTF fit resolution estimate better than 5.5 Å were used for processing. Templates for the Template picker were generated by 2D classification of particles obtained by the Blob picker from 250 micrographs. Particles obtained by the Template picker were subjected to several rounds of 2D classification and ab initio reconstruction. The final map was obtained with nonuniform refinement using C8 and C9 symmetry for octamer and nonamer, respectively. The resulting maps were used in model building.

### Results

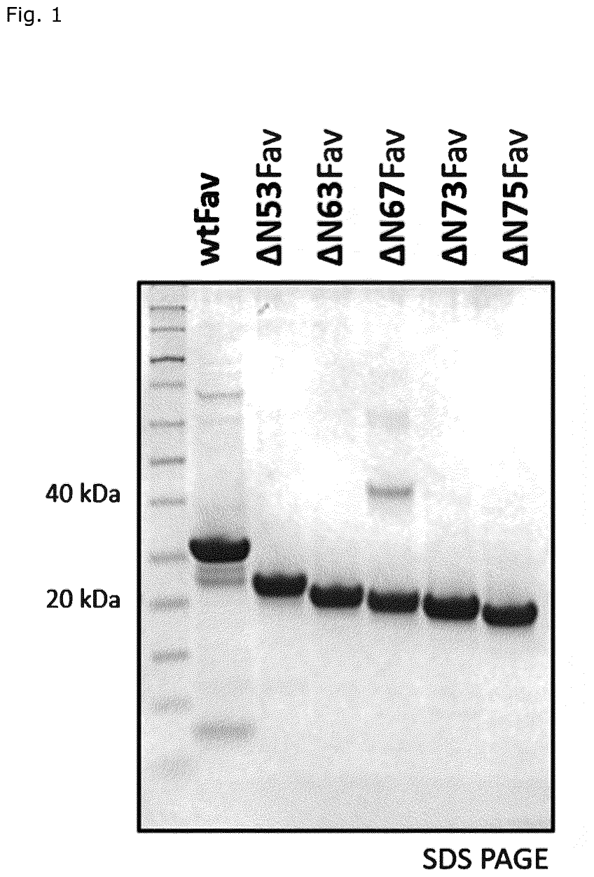

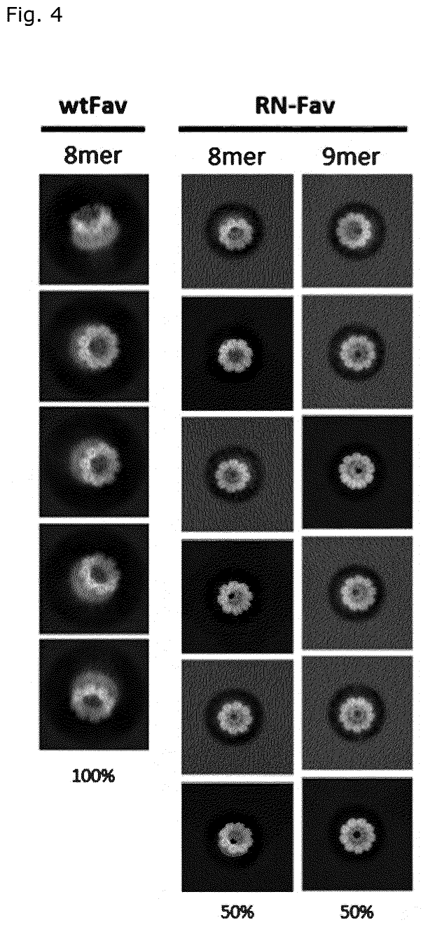

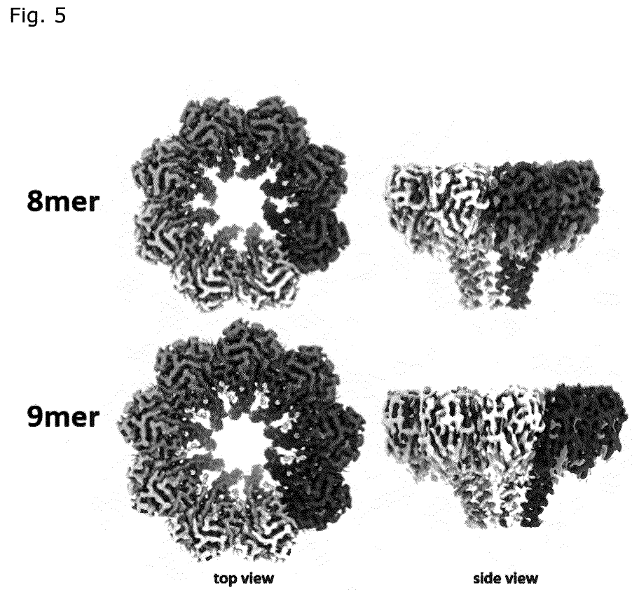

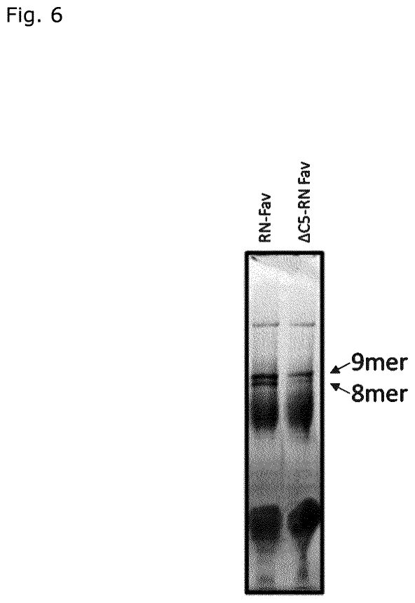

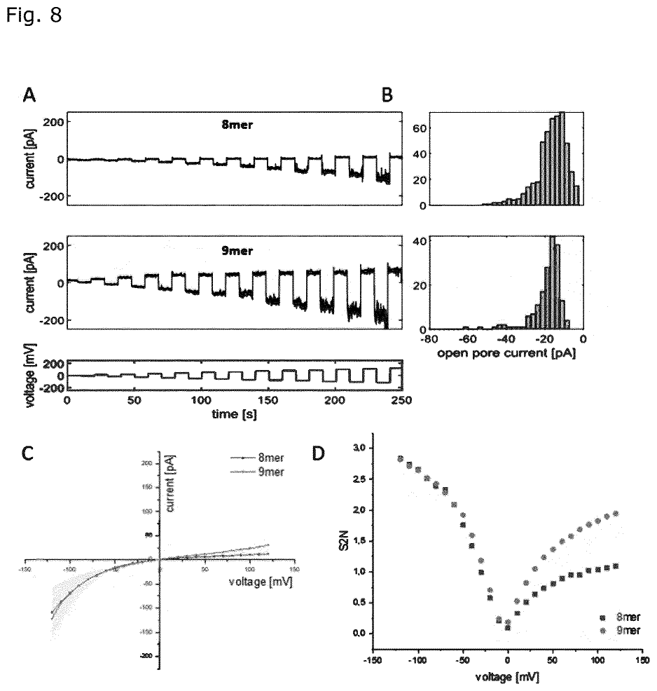

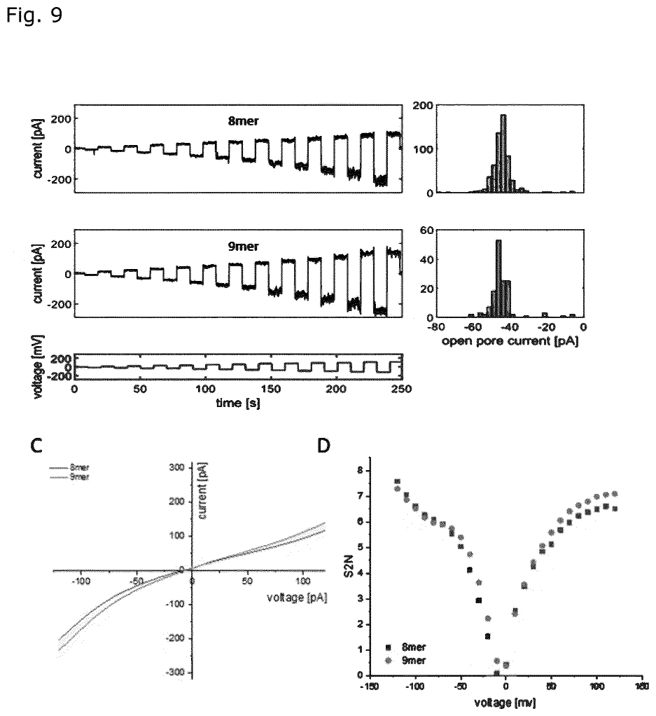

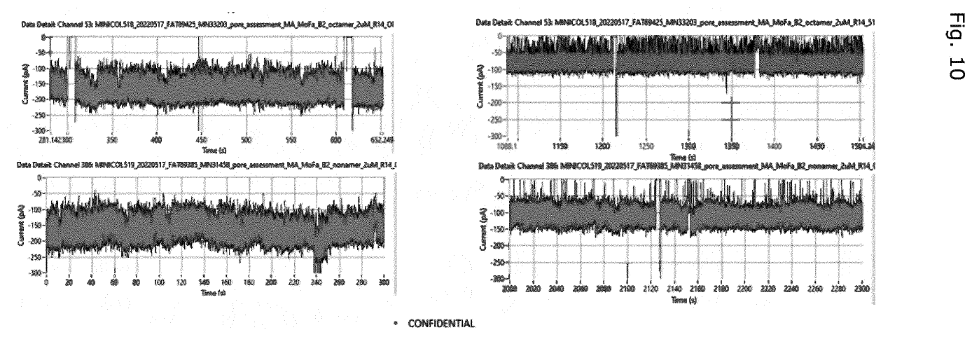

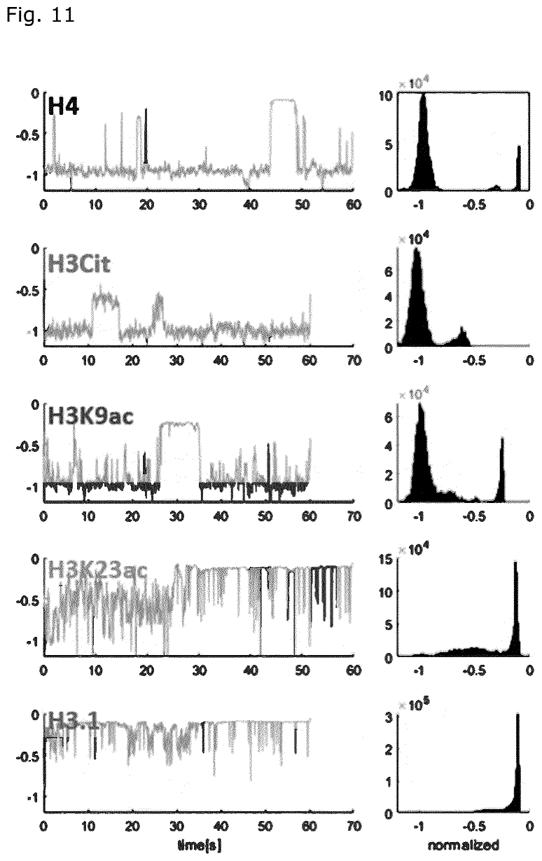

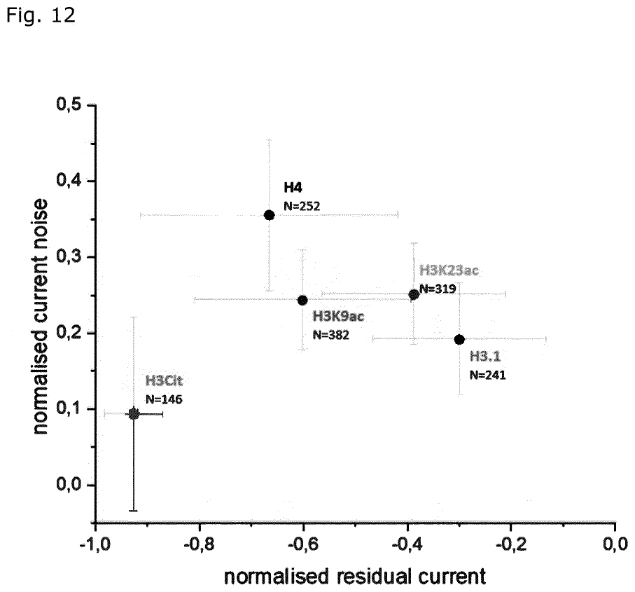

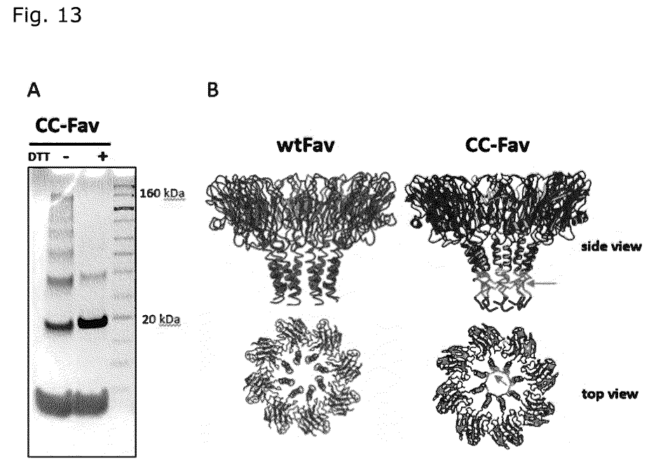

The results are shown in FIGS. 1-13 and explained in the Figure legends above.

### Example 2

### Introduction

Solid-state and biological nanopores, most commonly represented by transmembrane proteins or DNA origami, are nanometer-sized openings that traverse an electrically non-conductive membrane. [1] An applied potential across the membrane triggers the entry and potential translocation of the analyte through the pore, causing detectable specific current changes. [1b, 2] Such a nanopore approach has already been successfully applied for commercial ultra-long DNA sequencing [3] and has notable potential for proteomic applications. [1b, 4] Nevertheless, there are four main challenges that prevent the promising nanopore-based protein fingerprinting or sequencing approach from following commercially available DNA sequencing. First, distinguishing between 20 proteinogenic amino acids that are susceptible to various post-translational modifications is more difficult compared to the four basic core DNA bases. Secondly, peptides and proteins fold in a three-dimensional structure, which complicates the capture and translocation of molecules. In addition, unevenly distributed charges along the polypeptide chain may cause more complex interactions between the analyte and the pore, resulting in less controllable movement of the protein. Finally, the nanopore signals generated in the detection are much more complex and difficult to process and understand. To overcome these challenges, direct and indirect approaches have been explored to detect folded proteins with different molecular weights, [5] to track protein unfolding kinetics, [6] binding affinities [7] and various post-translational modifications (PTMs) such as ubiquitination, [8] phosphorylation, [9] acetylation, [10] propionylation, [11] and glycosylation, [9b] as well as to detect sequence variations [12] and amino acid chirality. [13]

Proteins play a key role in most biological processes and changes of their concentration in biological samples often correlate with diverse pathological conditions, making proteins suitable biomarkers alongside nucleic acids and small organic molecules. [14] Through intensive proteomic analyses in recent years, histones have gained attention as potential biomarker molecules. In addition to their physiological canonical function in DNA packaging and regulation of gene expression in the cell nucleus, histones also have extracellular functions. They have antimicrobial functions and can act as damage-associated molecular patterns (DAMPs) [15] and are a predominant protein in neutrophil extracellular traps (NETs). [15b, 16] Their elevated serum levels correspond, for example, to disease activity in patients with rheumatoid arthritis (RA), [17] cancer prognosis, [18] cardiac arrest, [19] urosepsis [20] and trauma. [15d] In contrast to established protein detection techniques such as mass spectrometry and immunoassays, particularly in the case of histones, nanopores could offer rapid, real-time, high-throughput detection of single molecules, especially when integrated into small portable devices. [3]

The most important prerequisite for optimal molecule detection is the compatibility of pore diameter and size of the analyte. [11, 21] Due to the large variety of proteins compared to nucleic acids, novel pores with the desired property are in high demand. Actinoporins, pore-forming toxins present in venom of sea anemones, [22] form pores by bundle of α-helices. [23] Pore formed by fragaceatoxin C (FraC), from sea anemone Actinia fragacea, was shown to be composed of eight identical protomers [23d] and was employed for nanopore sensing applications to discriminate nucleic acids, [24] detect PTMs in selected proteins [9b] and discriminate peptides of different length. [25] In the present work, we implemented a novel octameric actinoporin pore [26] for the detection and identification of medically relevant histone variants. We prepared a pore construct that stably inserts into robust polymeric MinION membranes, implemented in commercially available DNA sequencing devices, allowing us to perform high-throughput detection of different human histones. We were also able to quantify mixtures of two medically most important histones using a machine learning approach.

### Results and Discussion

wtFav Pore Optimization for Stable Insertion into Lipid and Polymeric Membranes

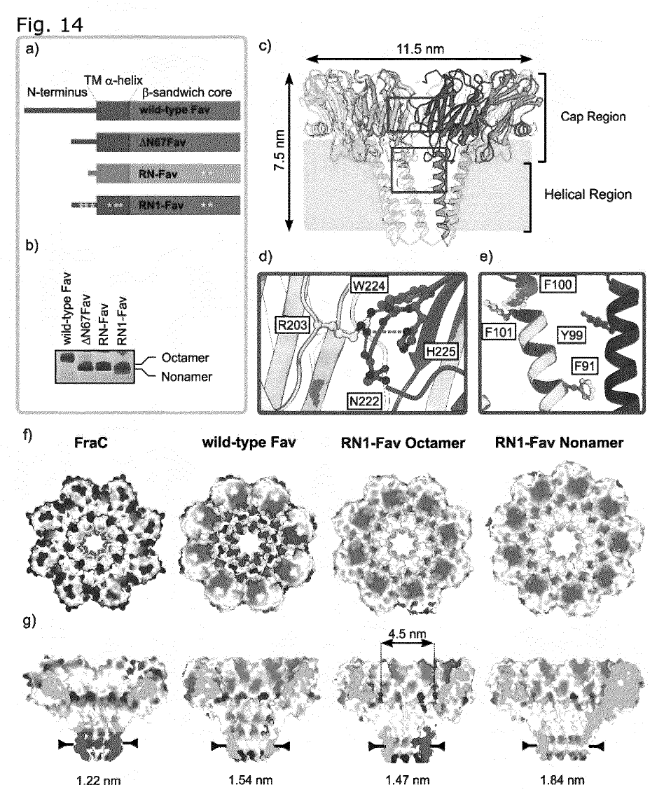

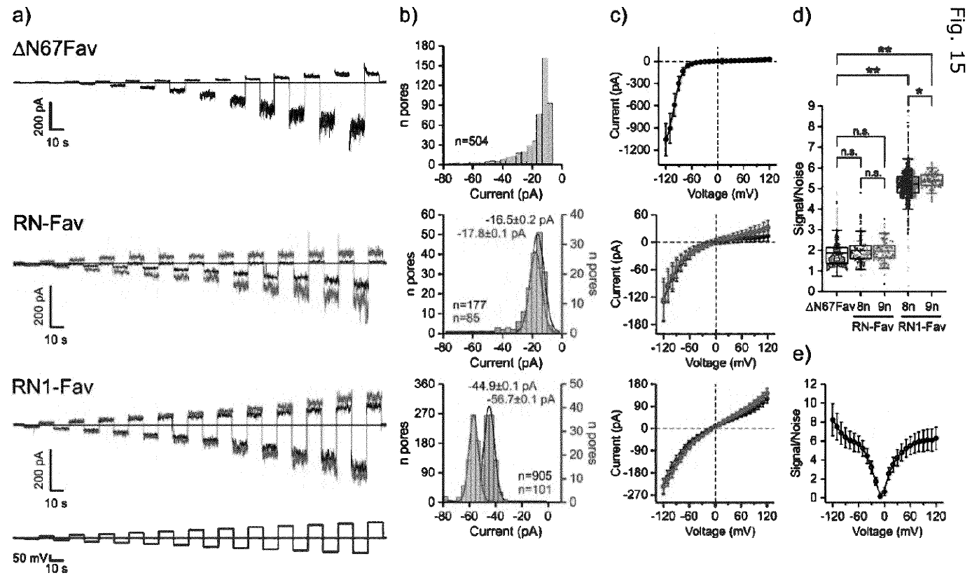

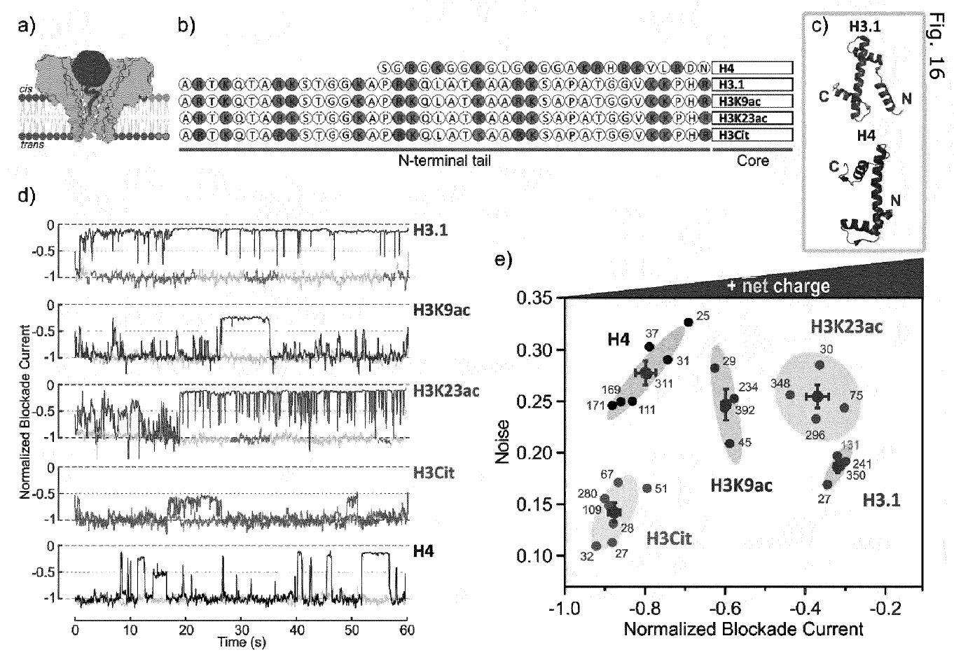

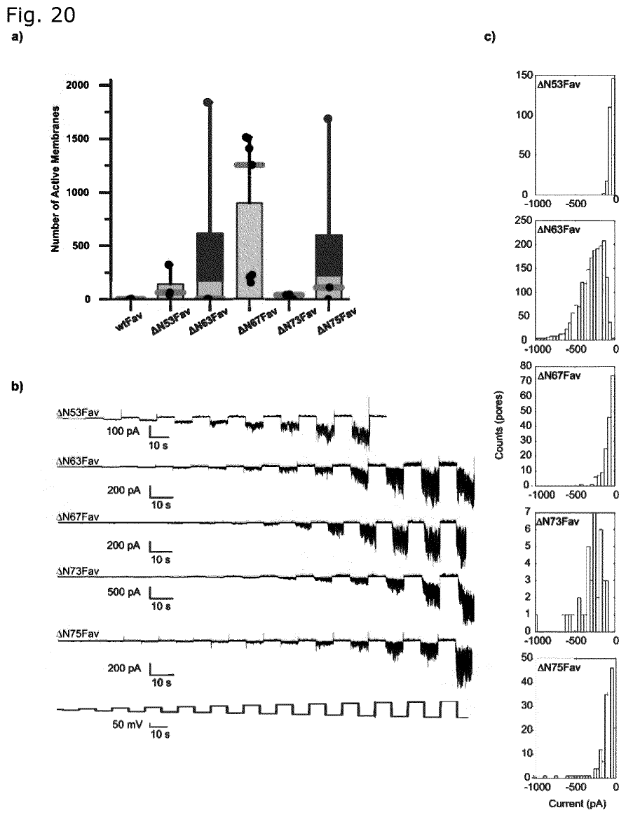

We have recently described a novel, conically shaped pore formed by an actinoporin-like homologue from the coral Orbicella faveolata (wild-type Fav, wtFav) (FIG. 19). [26] Compared to FraC pore, [23d] wtFav pores are similarly organized. The pore consists of two distinct regions. The cap on the cis side of the membrane is formed by circularly arranged 3-sandwiches of protomers. Each of these protomers also contributes a single α-helix to form the transmembrane region (FIG. 14a). wtFav has an additional, 82 amino acid long, unstructured N-terminus extending on the trans side of the lipid bilayer (FIG. 14a, FIG. 19). We attempted to incorporate wtFav pores into polymeric MinION membranes, but they could not be readily incorporated (FIG. 20). Efficient insertion was likely limited by the long unstructured N-terminus, which has been shown to inhibit the permeabilization activity of other actinoporins. [23a, 23c]

To address this issue, we made a series of constructs with different deletions of the N-terminus (FIGS. 19 and 20). All isolated constructs formed oligomers on large unilamellar vesicles composed of 1,2-dioleoyl-sn-glycero-3-phosphocholine (DOPC):sphingomyelin (SM) (1:1, mol:mol), and detergent-extracted pores could be inserted into polymeric MinION membranes (FIG. 20). The most promising variant, based on insertion efficiency into polymeric MinION membranes, was ΔN67Fav (FIG. 19), missing 67 N-terminal residues. The shortest construct tested, ΔN75Fav, which corresponds to the length of FraC (FIG. 19), showed unstable current and compared to other constructs, it exhibited a lower current signal with considerable noise, especially at applied negative voltages below −100 mV (FIG. 20b). We also observed an increased loss of pore signal after repeated measurements on the same flow cell (which was rarely observed with other constructs), presumably due to weaker insertion into the polymeric membrane. However, all tested constructs showed a broad distribution of open pore current at −90 mV (FIG. 20c), which prevented us from performing high-throughput experiments with single pores.

To improve the insertion of pores, the distribution of open pore currents and signal to noise ratio of Fav nanopores, we used the 3D structure of the wtFav pore to introduce point mutations using site-directed mutagenesis. [27] (FIG. 14a). The so-called RN-Fav construct corresponds to approximate size of FraC, beginning with R77 (FIG. 19), and in addition carried two mutations of the charged, non-conserved amino acids D203R and D215N (FIG. 14a). These mutations improved the current distribution in the open pore to a certain extent but not the signal to noise ratio (see below). RN1-Fav pore was, therefore, designed. Here, we used ΔN67Fav construct and included mutations D203R and D215N of RN-Fav. In addition, we replaced all positively charged residues in the unstructured N-terminus with negatively charged ones. This construct also contained four substitutions of amino acid residues with aromatic side chains in the transmembrane α-helical region to reduce the fenestrations between the α-helixes in the transmembrane region (FIG. 14e), which can be a potential source of the current noise (FIG. 19).

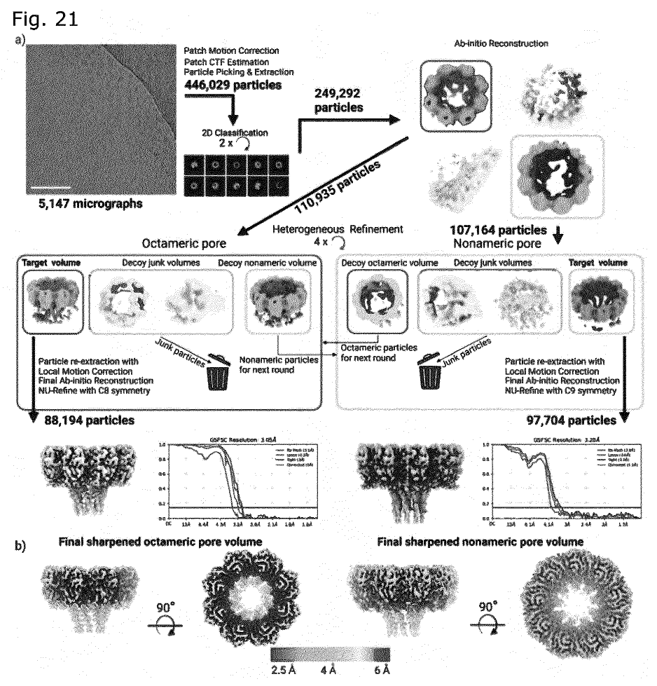

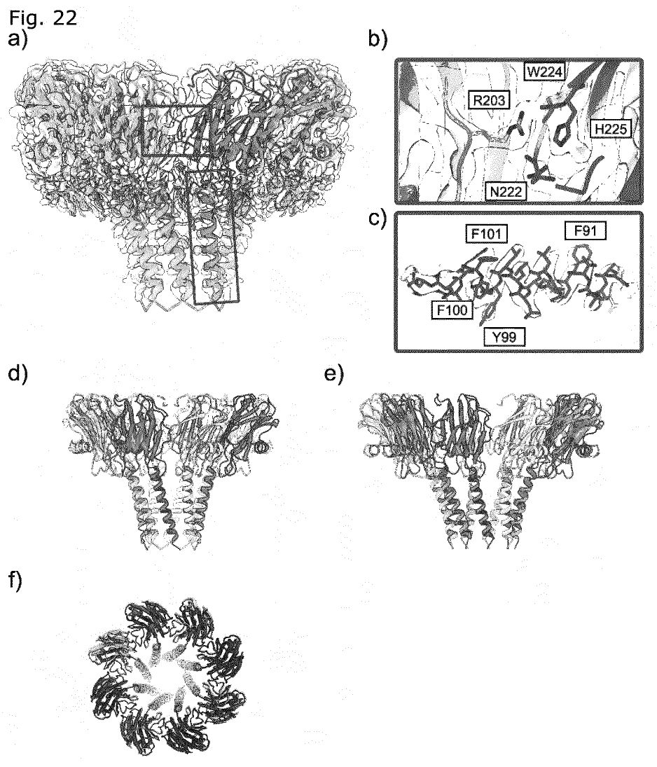

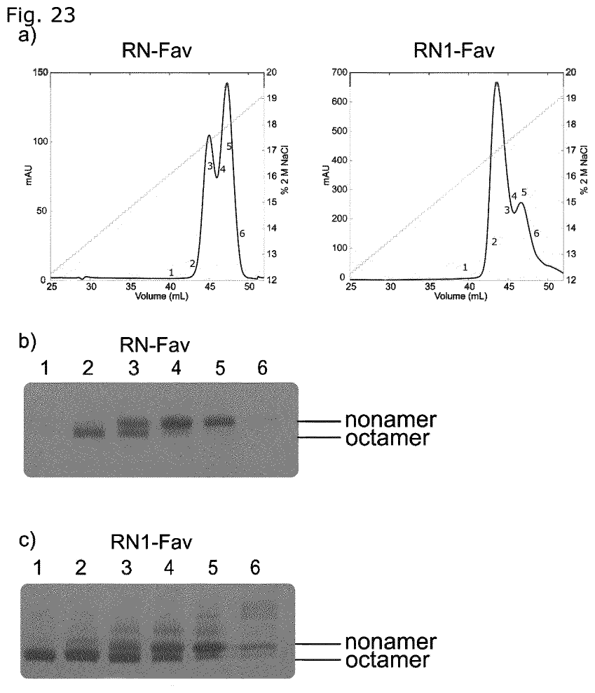

Compared to FraC, which mostly forms octameric pores [23d] and, under certain conditions, heptameric and hexameric pores (whose structure has not yet been determined), [25] wtFav oligomerizes into octameric and some nonameric pores (FIG. 14b), the latter of which are not stable when solubilized if the lipid membrane used for oligomerization does not contain cholesterol. [26] RN-Fav and RN1-Fav variants showed an altered octamer:nonamer ratio compared to ΔN67Fav (FIG. 14b, FIGS. 21 and 22). Due to the different net charge, due to difference in stoichiometry of protomers, homogeneous preparations of octameric or nonameric pores could be obtained by ion-exchange chromatography (FIG. 23). Cryo-electron microscopic reconstruction of octameric and nonameric RN1-Fav pores at 3 Å and 3.3 Å resolution, respectively (FIG. 14c, FIGS. 21 and 22, Table 1), revealed that the introduced D203R mutation enables the formation of additional stabilizing H-bonds between adjacent protomers in the cap region on the outer side of the pore. Two H-bonds are formed, the first between the side chain of R203 of one protomer and the main chain carbonyl group of N222 or the side chain of H225 of the other protomer. The second is formed by the side chain of R203 and the main chain carbonyl group of W224 (FIGS. 14c and d). In contrast, the side chain of aspartic acid at the same position in wtFav is too short to allow the formation of these H-bonds. These additional contacts on the outer side of the pore help stabilize nonameric pores once solubilized, similar to how the presence of cholesterol and more tightly packed lipids stabilize nonamers in the case of wtFav. [26] The RN1-Fav pores also differ in the diameter of the pore constriction compared to the FraC (diameter of 1.22 nm) and wtFav pores (diameter of 1.54 nm), with diameter of 1.47 nm and 1.84 nm for octameric and nonameric pores, respectively (FIG. 14e, FIG. 21). Due to the introduced mutations in RN1-Fav, the inner surface of the pore cap region is less negatively charged than in wtFav, but still much more negative than in the FraC pore (FIGS. 14f and 1g).

RN-Fav and RN1-Fav Pores Have Improved Characteristics in Comparison to wtFav Pores

Although most common single-channel recording devices provide an excellent basis for the electrophysical characterization of pores, [1a, 25, 28] they rarely achieve the desired high throughput that can be achieved with devices such as the MinION. Variants of Fav pores incorporated stably into MinION membranes (FIG. 20a), which enabled a desired high-throughput characterization. We characterized the RN-Fav and RN1-Fav pores in MinION flow cells and compared them with the ΔN67Fav pores. In all experiments, we used MinION flow cells and inserted them from the cis side of the membrane by employing a stepwise increasing voltage ramp from +30 to +300 mV. For the basic characterization of the pores, we used a voltage ramp with alternating voltage polarities from 0 mV to +120 mV (in 10 mV steps). Membranes with a single inserted pore were selected based on the constructed histogram of open pore current at −50 mV, which included all individual active pores on the MinION flow cell.

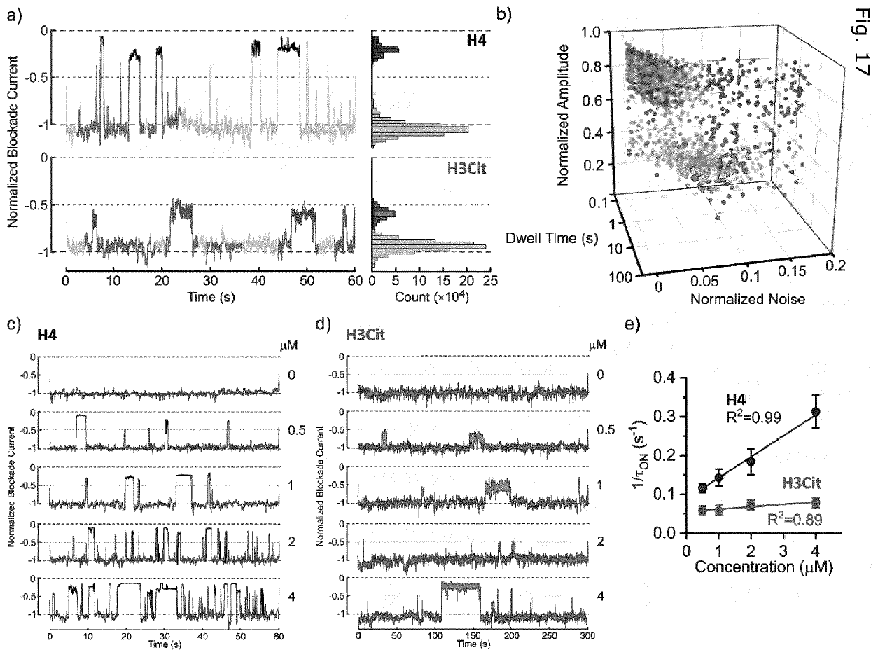

ΔN67Fav showed a broad distribution without recognizable, well-resolved peaks (FIG. 15a-c). In addition, the I/V curve was highly asymmetric and showed high and noisy currents at negative potential, but pore was almost closed at positive applied potential (FIG. 15c). In contrast, the changes introduced in the RN-Fav and RN1-Fav pores resulted in narrower current distributions (FIG. 15b), altered the shape of the I/V curves and increased the current at positive applied potential (FIG. 15c), and significantly increased the signal-to-noise ratio at −50 mV (FIG. 15d). In addition, both the nonameric RN-Fav and RN1-Fav pores showed higher open pore current compared to the octameric pores, due to the larger pore diameters (FIG. 14e).

The linearization of the I/V curve can be attributed to changes at the N-terminus of RN-Fav and RN1-Fav. The long and unstructured positively charged N-terminus of ΔN67Fav could clog the pore when a positive voltage is applied (FIG. 15b). Removal of a significant portion of the N-terminus in RN-Fav, which could not clog the pore, and additional mutations in RN1-Fav, which reduced the repulsion between the previously positively charged N-terminus and the positively applied voltage at the grounded electrode on the trans side of the membrane, resulted in more linear I/V curves and open pores at the positively applied voltages (FIG. 15c). The effect was more pronounced for RN1-Fav pores compared to RN-Fav pores (FIG. 15c).

The RN1-Fav pores exhibited a significantly better signal-to-noise ratio compared to ΔN67Fav and RN-Fav pores, which was determined as open pore current (I0)/standard deviation of open pore current at −50 mV. The substitution of positively charged amino acids in RN1-Fav affected the protonation of the pore lumen (FIGS. 14f and g). The effect of charge distribution alongside the pore lumen on signal-to-noise ratio was already reported for OmpF [29] and MspA. [30]

Overall, the improved open pore current distribution together with the increased open pore current at negative voltage and significantly increased signal-to-noise ratio made the RN1-Fav pores (FIGS. 15d and e) the most promising candidates for protein detection and identification purposes.

### RN1-Fav Pores Enable Identification of Human Histone Variants

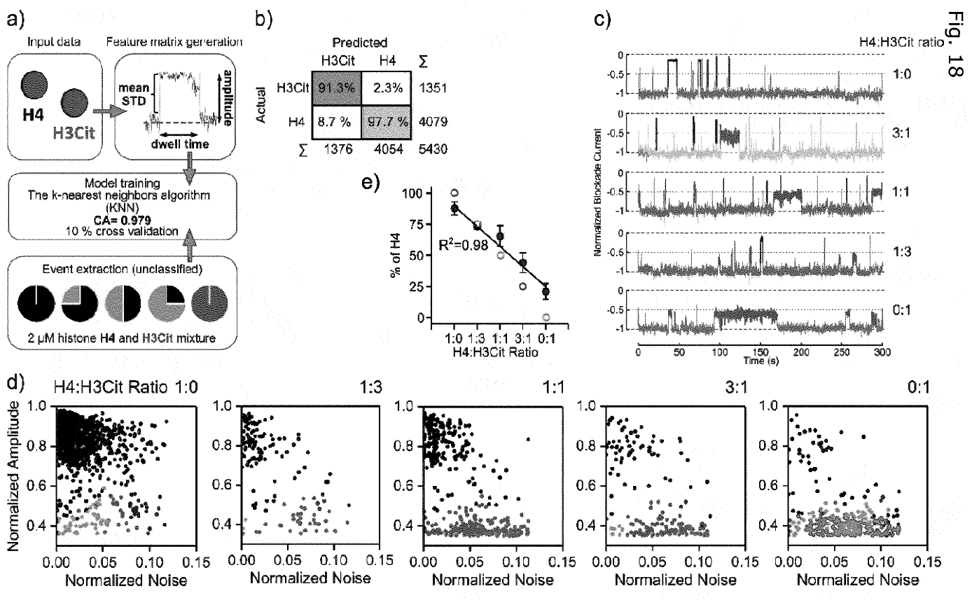

Since the RN1-Fav pores stably insert into the MinION membranes and showed improved electrophysical properties compared to the ΔN67Fav and RN-Fav pores, we tested the octameric RN1-Fav pores for detection and identification of human histone variants. We chose octameric pores because we achieved a higher number of single inserted pores compared to nonameric pores (FIG. 15b) and the fraction of octameric pores after ion-exchange chromatography was more homogeneous (FIG. 23). It has already been shown for derivatized single amino acids that the non-covalent interactions between the lumen of the nanopore and the analyte influence the ion mobility through the pore. [31] Based on the data showing that the charge of the pore is considered one of the most important parameters for controlling analyte capture and translocation, [32] we hypothesized that negatively charged RN1-Fav pores (FIGS. 14f and g) would be able to discriminate positively charged human histone variants (FIG. 16a) depending on the net charge of the unstructured N-terminal histone tail. Histones are approximately 15 kDa molecules with an ordered globular domain and an unstructured N-terminal tail (FIG. 16a-c). The N-terminal tails are susceptible to PTMs (FIG. 16b). [33] Histone H4 (net charge: +17.3, N-terminal tail net charge +7.3) has a 24 amino acid N-terminal tail and core domain of 79 amino acids organized in four α-helices (FIG. 16c). Histone H3.1 (net charge: +19.4, tail net charge +12.6) is slightly larger and composed of 136 amino acids. It has 39 amino acids long N-terminal tail and 97 amino acids core domain composed of four α-helices (FIG. 16c). Since histones are often post-translationally modified, we also characterized histones H3K9ac (net charge: +18.4, tail net charge +11.6) and H3K23ac (net charge: +18.4, tail net charge +11.6) with a single acetylated lysine residue at positions 9 and 23, respectively, and H3Cit (net charge: +11.4, tail net charge +7.6) with five citrullinated arginine residues in the N-terminal tail and three citrullinated arginine residues in the globular domain (FIG. 16b). The dimensions of the histone core domain are approximately 5.9×3.8×2.5 nm for H3.1 and 5.9×2.9×2.3 nm for smaller H4 (estimated with ChimeraX [34]). Since the width of the pore RN1-Fav octameric pore lumen is approximately 4.5 nm (FIG. 14g), these dimensions would allow the histones to be captured into the pore from the cis side.

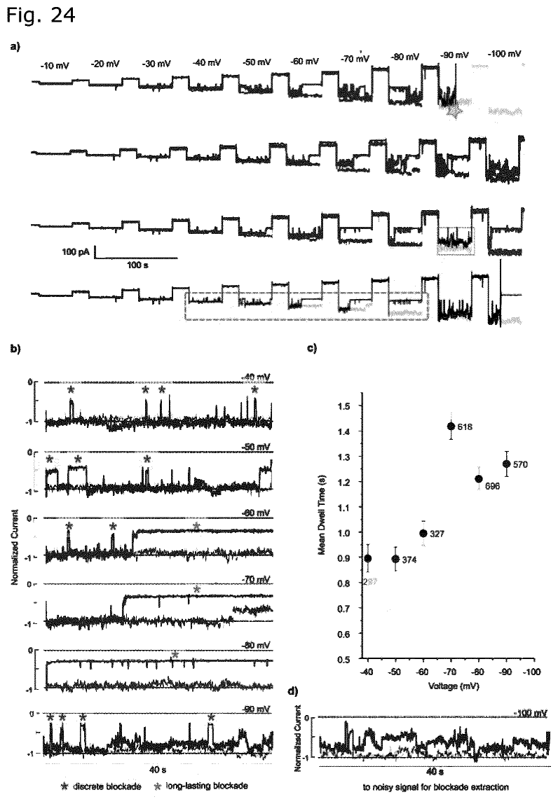

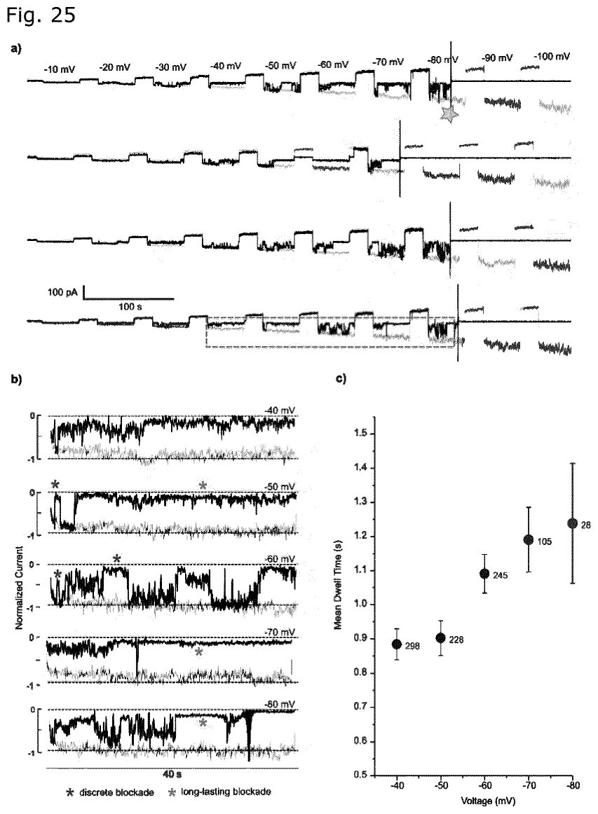

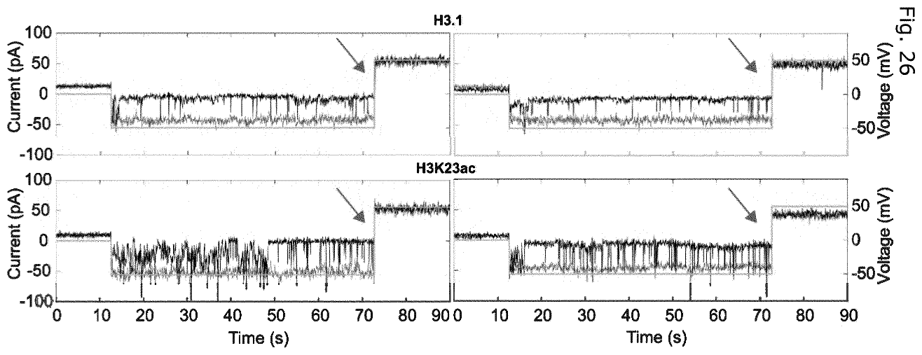

All experiments with histones were performed at −50 mV, as the histones are positively charged and the electrophoretic force would therefore help to trap them in the negatively charged lumen of the pore. [35] The capture started at voltages below −30 mV. In the case of H4, the smallest histone based on molecular weight, most pores showed well-resolved discrete blockades at −40 and −50 mV, but at higher voltages the blockades became longer and often only changing the voltage polarity restored the open pore current (FIG. 24).

Interestingly, after applying −90 or −100 mV, some pores remained open and only a few discrete blockades occurred, but the high current noise prevented us from accurately determining the blockades (FIG. 24). Similar, but with longer lasting pore occupancies even at lower voltages was observed for larger and more positively charged H3.1. Furthermore, at voltages lower than −80 mV, membranes with inserted pores rarely remained unbroken (FIG. 25). Thus, the most likely explanation is that we observe histone trapping at lower voltages, while higher voltages could possibly trigger histone translocation with concurrent destabilization and disruption of transmembrane α-helical bundle of RN1-Fav leading to high current noise. In addition to the clear distinction of blockades at −50 mV, another reason for recording histone capture at −50 mV was that the signal-to-noise ratio increases dramatically up to −50 mV and does not improve significantly at higher negative voltages (FIG. 15e).

In the presence of 2 μM histone variants at applied voltage of −50 mV, blockades of pores occurred in all histone samples, however, with different patterns (FIG. 16d). After the addition of histone H3.1, the pores exhibited high current noise at the beginning of the one-minute trace, and soon thereafter the blockade current reduced almost completely, with some spikes observed. Similar traces were observed for the acetylated histone variant H3K23ac, but with more frequent negative spikes. In both cases, the open pore current recovered after applying voltages of opposite polarity (FIG. 26), further confirming that histones were trapped in the pore and ejected from the pore when the voltage polarity was reversed.

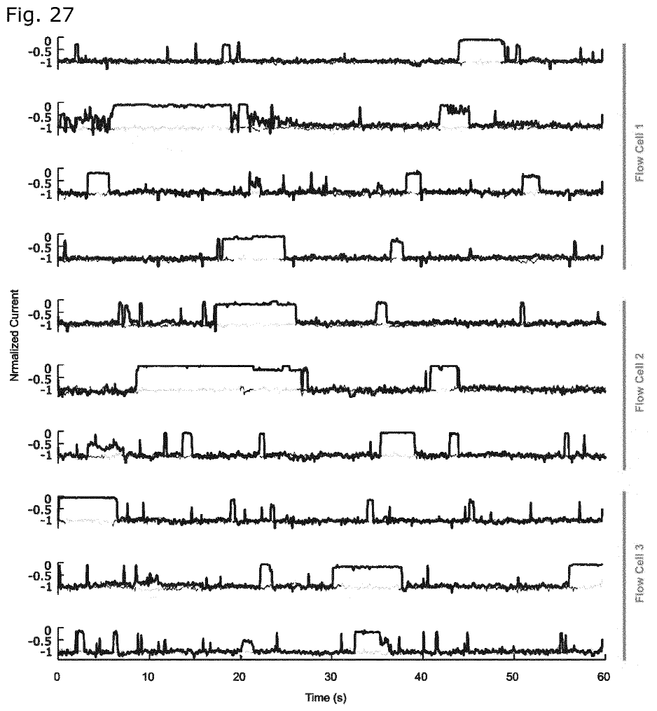

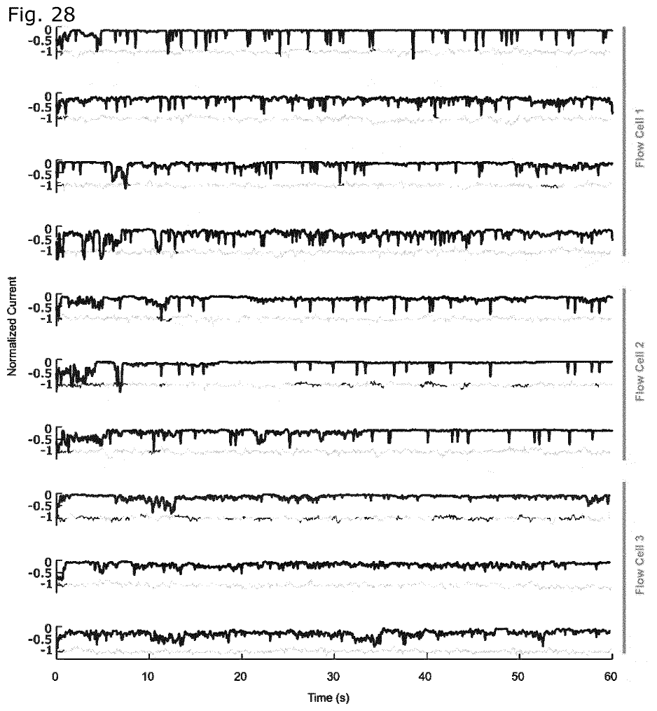

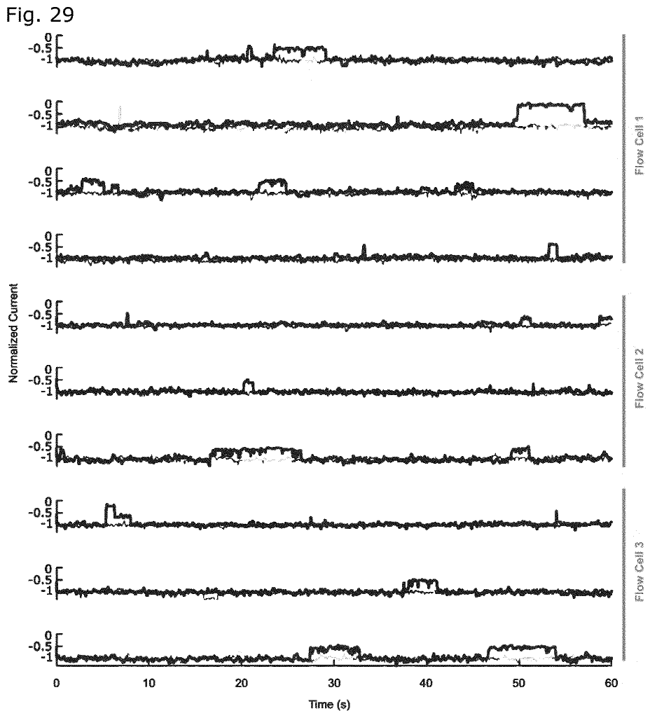

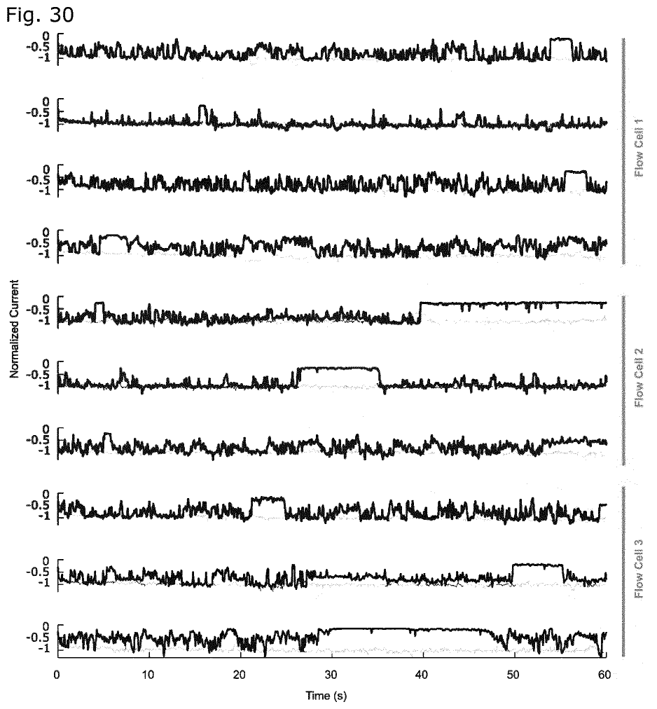

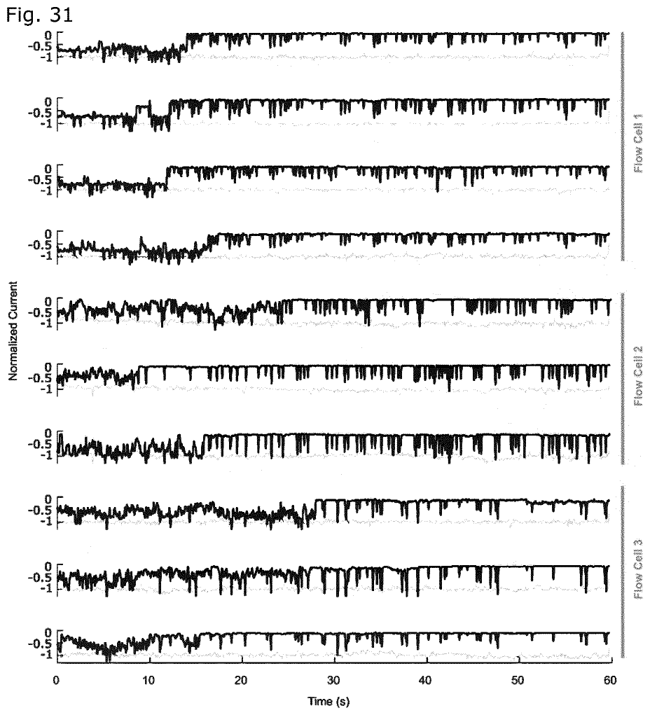

In contrast, the presence of 2 μM histone H3K9ac resulted in higher current noise but also showed discrete blockades, almost no long-lasting reductions in open-pore current were observed and negative spikes disappeared. Similarly, the addition of histone H3Cit and H4 resulted in discrete blockades without negative spikes (FIG. 16d, FIGS. 27-31).

Qualitatively different traces for five different histone samples (FIG. 16d) enabled a bulk analysis on large pore numbers. We calculated the average blockade current of individual pores from at least four different flow cells (FIG. 16e, see raw data traces in FIGS. 27-31) over the entire duration of the experiment (60 s, unless otherwise stated) and its standard deviation of the signal, referred to as current noise. The averaged blockade current in the presence of the histones roughly correlated with the net charge of the histones (FIG. 16e). The addition of H3.1 with the highest positive charge resulted in the lowest averaged normalized blockade current (−0.32±0.01) and the pores were blocked for almost the entire measurement period (FIGS. 16d and e). A similar long-lasting pore blockade was observed for H3K23ac with the higher averaged normalized blockade current (−0.37±0.03), but the long-lasting-blockades occurred with a time delay compared to histone H3.1 (FIG. 26). Interestingly, acetylation at position further away from the core region in H3K9ac reduced the long-lasting blockades and resulted in an averaged normalized blockade current of −0.60±0.01. H3Cit with the lowest net positive charge resulted in the appearance of discrete blockades with an average normalized open pore current of −0.88±0.02 and almost no long-lasting pore blockades (FIGS. 16d and e). The smaller H4 showed similar relative high current of −0.80±0.03, While such real-time bulk analysis of tens to hundreds of pores simultaneously trapping the full-length histones did not provide a clear correlation between the observed noise of the current and histone net charges, it still allowed clear discrimination of histone variants by a combined analysis of relative blockade current and noise (FIG. 16e).

Current Blockade Analysis Differentiates Histones with a Single Amino Acid Modification

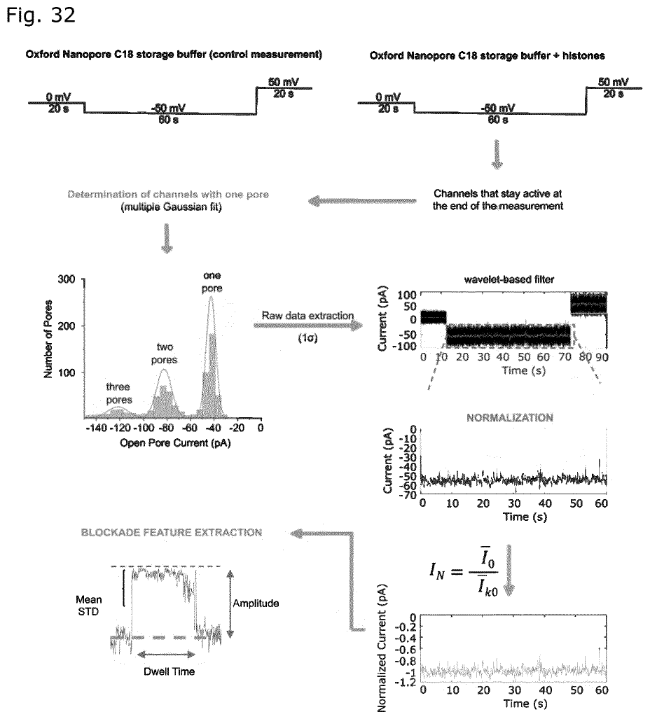

Although bulk analysis allowed us to distinguish between histones when testing a single histone variant at a time, we then performed a detailed blockade analysis that allowed us to discriminate mixtures of histone variants. The blockades amplitude (determined as ΔI/I0), the noise (standard deviation of the blockade current (Ih)) and the dwell time (FIG. 32) were calculated for the blockings extracted by the threshold method. ΔI is a difference between I0 and Ih. I0 and Ih were defined as the relative open pore current at min −50 mV and the mean blockade current, respectively. The blockades extracted from different MinION flow cells in the presence of a particular histone were grouped and further analyzed.

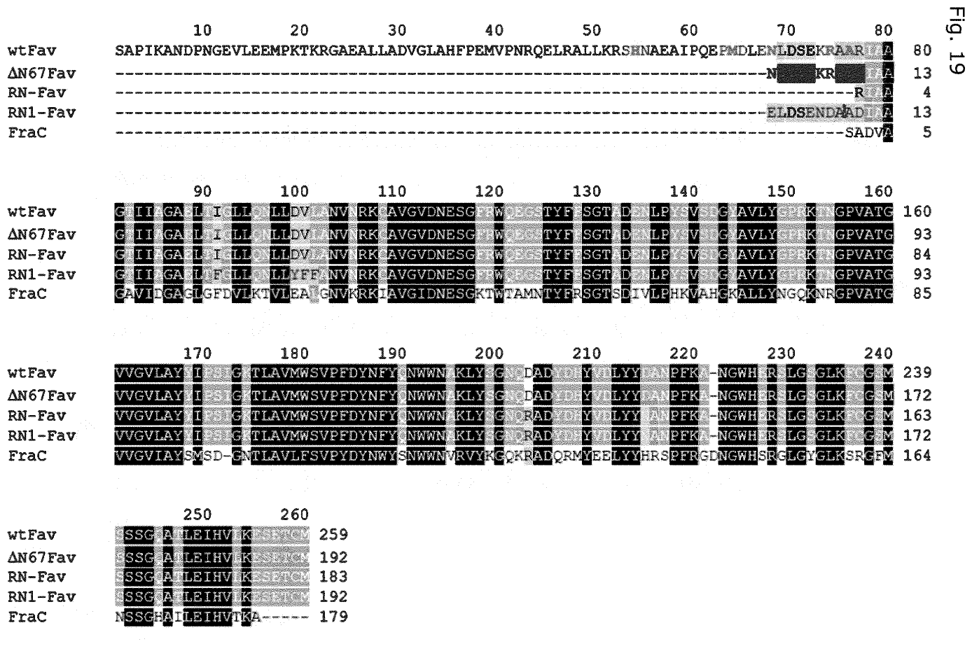

Due to the medical relevance and presence in serum samples, [17, 19, 37] and their clear separation based on the results of the bulk analysis (FIG. 16e), we focused on analyzing blockades caused by H4 or H3Cit (FIG. 17a). The highest blocking amplitude (0.83±0.10) was observed for the shortest histone variant H4, while H3Cit caused blocking with smaller amplitudes (0.38±0.08). Most likely, due to its smaller size, H4 could be trapped deeper into the pore lumen and thus hinder the current flow through the pore more than H3Cit, which is probably also exposed to a lower electrophoretic force due to its lower net positive charge.

Significant differences were also observed in the blockade noise, which corresponds to blockade current fluctuations. Histone H4 caused blockades with the lower noise (0.012±0.036) compared to histone H3Cit (0.057±0.065), that was most probably exhibited weaker electrostatic interactions with negatively charged pore surface in the lumen. Similarly, significant differences were observed when inspecting the blockade dwell times (FIG. 17a). Here H3Cit caused longer blockades than H4 (FIG. 17a). When plotted in 3D space based on the blockade's amplitude, noise and dwell time (FIG. 17b), we were able to clearly discriminate between two important extracellular histone variants H4 and H3Cit.

In addition, we tested whether RN1-Fav pores would also allow us to quantify histones based on their blockade frequency. In a series of experiments on the MinION flow cells with increasing concentrations of H4 (FIG. 17c) or H3Cit (FIG. 17d) (at a concentration of 0.5, 1, 2 or 4 μM), the observed blockade frequency for both histones, determined as 1/TON (time between the blockades), correlated linearly with histone concentration (FIG. 17e). Importantly, even a short measurement, 3 min in the case of H3Cit, enabled quantification of the histones and thus showed great potential for rapid real-time detection of histones compared to the established, longer duration detection methods

### RN1-Fav Pores Allowed Discrimination of Medically Relevant Histone H4 and H3Cit in a Mixture

In a real biological sample, histones rarely occur in a single variant. Therefore, we tested whether RN1-Fav pores allow the discrimination of histone H4 and H3Cit variants in a mixture using machine learning. Machine learning has recently been adapted to analyze nanopore events, [38] while providing fast and automatic predictions based on the raw data. At the same time, it avoids possible human bias, especially when working with more complex data sets. The input data used to train and test the model were the extracted blockades from the previously mentioned blockade current analysis collected from several independent flow cells (FIG. 18a).

The model based on the k-nearest neighbors (kNN) algorithm resulted in a classification accuracy of 0.960 (FIG. 18a). The same blockade extraction procedure was also applied to the traces obtained after applying H4 and H3Cit in different molar ratios (1:0, 3:1, 1:1, 1:3 and 0:1) at a final histone concentration of 2 μM (FIG. 18a, c). The assessment of the predictive capacity of the obtained model was based on the number of blockades categorized as H4 or H3Cit blockade (FIGS. 18c and d) and showed a clear linear dependence (FIG. 18e).

### Conclusion

Nanopores are able to detect methylated and acetylated histone H4 peptides [10] or the whole nucleosome complex. [40] Hereby, we developed a RN1-Fav protein nanopore, which is based on the actinoporin homologue from Orbicella faveolata and capable of high-throughput label-free detection of full-length human histones. RN1-Fav nanopore can stably insert into MinION membranes, making it a good candidate for the development of biosensing applications where immediate on-site detection of analytes is required, such as biomedical applications. RN1-Fav pore extends a toolbox of nanopores for high-throughput sensing, alongside CsgG/F, [41] phi29 channel [42] or lysenin pores [43] that were analyzed using MinION platform. Due to the high negative charge of the nanopore surface exposed to the pore lumen, the RN1-Fav pores offer the possibility to detect positively charged proteins. As proof of principle, we have detected the medically relevant full-length histone proteins H4, H3.1 and their post-translationally modified variants. In addition, we were able to quantify and discriminate two important extracellular histones H4 and H3Cit in mixtures using machine learning. While histone proteins are increasingly emerging as potential biomarkers, our results represent a new step toward the development of fast, accurate, single molecule method for a real-time monitoring of histone proteins in human body samples.

### Materials and Methods

### Materials

Lipids, sphingomyelin (from porcine brain; SM), 1,2-dioleoyl-sn-glycero-3-phosphocholine (DOPC) and 1,2-diphytanoyl-sn-glycero-3-phosphocholine (DPhPC) were from Avanti Polar Lipids (Alabaster, AL, USA). Isopropyl β-D-1-thiogalactopyranoside (IPTG) and kanamycin were from Gold Biotechnology (USA). Other chemicals were from Sigma-Aldrich (USA) unless otherwise specified.

We used the following histones: H3.1 (Human H3-WT (3.1), The Histone Source, Colorado State University, USA), H3K9ac (Recombinant Histone H3K9ac (EPL), Active Motif, USA), H3K23ac (Recombinant Histone H3K23ac (EPL), Active Motif, USA), H3Cit (Citrullinated Histone H3 (human, recombinant), Cayman Chemical, USA), H4 (Human H4, The Histone Source, Colorado State University, USA).

### Strains and Protein Expression Constructs

The nucleotide sequence for Fav constructs were inserted into a modified pET28a (+) plasmid with the N-terminal 6×histidine tag and TEV restriction site (ENLYFQGHM or ENLYFQS) and were used for transformation of E. coli DH5a. The following amino acids remain at the N-terminus of the mature proteins after cleavage with TEV: GHM in the wtFav and Δ67NFav or S in the case of RN-Fav and RN1-Fav (see the sequences of the mature proteins in FIG. 19). SEQ ID NO: 1 lacks GHM at its N-terminus.

### Expression End Purification of Recombinant Proteins

100 μL of competent cells E. coli BL21(DE3) strain was transformed with the plasmid, applied to agar plates with kanamycin resistance and incubated overnight at 37° C. A single colony was inoculated into 10 mL of the LB medium (supplemented with 30 μg/mL kanamycin) and incubated overnight at 37° C. and 180 rotations/min. The overnight culture was then added to 1 L of TB medium (30 μg/mL kanamycin) and incubated at 37° C. until reaching A600=0.8, when we induced protein expression with 0.25 mM IPTG. 16-18 h post induction (incubated at 18° C.) we harvested cells by centrifuging biomass at 4000×g for 10 min and at 4° C. The cell pellet was washed with PBS (1.8 mM KH2PO4, 137 mM NaCl, 10 mM Na2HPO4, 2.7 mM KCl, pH 7.4), sonicated 2×7 min (1 s on, 2 s off) and centrifuged at 40 000×g and 4° C. for 40 min. A soluble fraction was filtered through 0.22 μm polyethersulfone filter and applied to the Ni-NTA column (Qiagen, Germany), washed with PBS and eluted with PBS containing 0.5 M imidazole. Following overnight TEV digestion and dialysis against PBS, cleaved proteins were isolated by using another Ni-NTA chromatography step.

### Large Unilamellar Vesicle (LUV) Preparation

Multilamellar vesicles composed of SM and DOPC in a 1:1 molar ratio at a 50 mM lipid concentration were prepared by dissolving lipids in chloroform (analytical standard, Sigma-Aldrich, USA). After removing solvent by using rotavapor and formation of thin lipid film, we resuspend the film in PBS by vortexing, followed by six freeze and thaw cycles. LUVs of 100 nm in diameter were prepared by a single-use NanoSizer Extruder (T&T Scientific, South Korea).

### Pore Isolation

150 μL of LUVs at 50 mM lipid concentration were mixed with soluble monomeric protein in a molar ratio 1:300 (protein:lipid) and incubated at 37° C. for 30 min. Vesicles were disrupted by adding lauryldimethylamine oxide (LDAO) to a final 0.75%, diluted in 50 mL of 50 mM Tris/HCl, 0.02% Brij 35, pH 8 and further purified by ion exchange chromatography using 50 mM Tris/HCl, 2 M NaCl, 0.02% Brij 35 (resource Q, Cytiva, USA) as an elution buffer, employed in a linear salt gradient. Pores eluted at app. 0.3-0.5 M NaCl.

### Native PAGE

17 μL of pore sample (after ion exchange chromatography) were mixed with 6 μL of native PAGE loading buffer (50 mM Bis/Tris, 6N HCl, 50 mM NaCl, 10% w/v Glycerol, 0.001% Ponceau S, pH 7.2) and G-250 (final concentration 0.02%), centrifuged for 10 min at 16 000×g and loaded on 4-16% Bis/Tris gels. The running buffer was composed of 0.05 M Bis/Tris, 0.05 M tricine pH 6.9, and the cathode buffer was prepared by mixing 190 mL of the running buffer with 10 ml of mL 20× NativePAGE™ Cathode Buffer (Invitrogen, USA). After fixation in 40% methanol and 10% acetic acid solution, gels were washed in 8/a acetic acid solution.

### Cryo-EM Sample Preparation and Data Acquisition

3 μL of pore samples with concentration −1 mg/mL was applied to glow-discharged (GloQube® Plus, Quorum, UK) Quantifoil R1.2/1.3 mesh 200 copper holey carbon grid (Quantifoil, Germany), blotted under 100% humidity at 4° C. for 6-8 s and plunged into the liquid ethane with a Mark IV Vitrobot (Thermo Fisher Scientific, USA). Micrographs of RN1-Fav pore were recorded on Glacios (Thermo Fisher Scientific, USA) with a Falcon 3EC direct electron detector (Thermo Fisher Scientific, USA) operated at 200 kV using the EPU software (Thermo Fisher Scientific, USA) at a nominal magnification of 190 000× and a pixel size of 0.745 Å.

### Cryo-EM Data Processing

All steps of data processing for all samples were performed in cryoSPARC v4 [44] with built-in algorithms, the workflow is summarized in FIG. 21a. Movies were aligned and dose-weighted with patch motion correction, contrast transfer function (CTF) was estimated with patch CTF. Micrographs with estimated CTF fit above 6 Å were excluded from further analysis. Initially, particles were picked by hand to generate 2D templates for template picking. Particles were extracted and underwent two rounds of 2D classification. Particles from the best 2D classes were used in ab-initio reconstruction to generate 4 volumes. Among these 4 volumes, one corresponded to octameric and one to nonameric pore. Octameric and nonameric particles were separated and cleaned up by four iterations of two parallel heterogeneous refinements with 4 volumes each. In each round, one volume corresponded to the pore of target stoichiometry, octameric or nonameric, respectively. In addition, to the target volume, three decoy volumes were used. One of the decoy volumes was a volume of the other stoichiometry, e. g. if octameric pore was a target, one of the decoy volumes/was the nonameric pore, and vice versa, while the other two were random volumes generated during ab-initio reconstruction. After each round, particles classified into the two junk decoy volumes were discarded and particles classified to the alternative stoichiometry were moved to the next step of the parallel refinement. For example, after the first round of heterogenous refinement, particles that classified to the volume of the nonameric pore where the target volume was octameric, were combined with particles that classified to the nonameric volume in the process where the target was also nonameric and vice versa. These two new particle stacks of octameric and nonameric pores were then used in the next round of heterogeneous refinement. After heterologous refinement, particles from the best 3D class were re-extracted using local motion correction and used in homogeneous refinement and subsequent non-uniform (NU) refinement with applied C8 or C9 symmetry.

### Model Building

Atomic models of protomers were built with the iterative use of Coot [45] and Isolde plugin [46] for ChimeraX. [47] Phenix [48] was used for applying symmetry and restraint calculation for lipid molecules. The pore structure of wtFav monomer was used as the starting model. Each protomer (subunit) in a pore is identical to the rest. This is true for both types of pores. For octameric RN1-Fav pore, model was built from Ile 78 to Met 259. In addition to the protein, 6 lipid (sphingomyelin) molecules per protomer (48 in total) were placed in the model. For nonameric RN1-Fav pore, the protein model was built from Ala 79 to Met 259. In addition to the protein, 5 lipid (sphingomyelin) molecules per protomer (45 in total) were placed in the model. Details of data acquisition and refinement statistics are shown in Table 1.

### MinION Experiments

For pore insertion into polymeric membranes, we used empty MinION flow cells provided by Oxford Nanopore Technologies (United Kingdom), MinION set up, Oxford Nanopore C18 storage buffer and 20 kHz sampling frequency. Prior to pore insertion, each flow cell was washed with 1 mL of C18 buffer and inspected by membrane quality control measurements. For pore insertion 1-10 μL of pore sample with a concentration of 0.015 mg/mL was diluted into a final 1 mL of C18 buffer. Pore insertion was performed by adding 300 μL of diluted pore sample via the sample port and running the MinKNOW pore insertion script. For I/V curve measurements we used a voltage protocol starting at 0 mV and increasing by ±10 mV steps to reach ±120 mV. Before applying histone proteins to inserted pores, we measured open pore current (in the C18 buffer) at 0 mV (10 s), −50 mV (60-300 s) and +50 mV (20 s). The same voltage protocol (if not stated otherwise) was run after applying 100 μL of (0.5-4 μM) histones diluted in a C18 buffer. At the end of the measurement, the cells were washed with 1 mL of C18 buffer and the same voltage protocol was applied again to confirm the removal of histone proteins from the flow cell. If measuring multiple concentrations or mixtures on the same flow cell, the washing step was applied after each measurement.

### MinION Data Analysis

The analysis of data obtained by MinION reads was done by our own MATLAB R2022b (MathWorks, USA) script. Before calculating the mean current for I/V curves and its standard deviation, for a specific pore at a specific voltage, the data were down-sampled 100 times. To analyze the blockade events in the presence of histones, we determined the pores that stayed active along the whole measurement. The open pore currents (at −50 mV) (Ik0) for pores were plotted as a histogram for which we employed the Gaussian fit. Pores that were within one standard deviation from the peak of the fit were used for the event detection. All traces were filtered using the wavelet-based filter and no down sampling was applied. To normalize the data, the control traces and the traces in the presence of histones were divided by the mean open pore current at −50 mV of the control measurement (Ik0). The bulk analysis data were calculated by averaging the mean current and its standard deviation (noise) at −50 mV for all pores included in analysis. To gain the blockade parameters we used the threshold-based method, where the current had to exceed 4.5 standard deviations above the open pore current standard deviation, and the change had to be at least 5 ms long (the minimal dwell time). For each event, its mean current (blockade amplitude, ΔI/I0, ΔI=I0−Ih, where I0 is the relative open pore current at −50 mV and Ih is mean current of the blockade), its standard deviation and the dwell time were calculated. Data were plotted by MATLAB R2022b and Origin 2018 (OriginLab, USA).

TABLE 1
Cryo-electron microscopy data collection,
processing and model validation statistics.

| Data collection | | |
|---|---|---|
| Microscope | Glacios | |
| Magnification | 190,000 | |
| Voltage (kV) | 200 | |
| Detector | Falcon 3EC | |
| Total electron dose (e⁻/Å²) | 32 | |
| Defocus range (μm) | −0.4~−2.8 | |
| Pixel size (Å) | 0.745 | |
| Number of micrographs | 5,147 | |
| | Octameric | Nonameric |
| Data processing | | |
| Initial particle number (all) | 446,029 | |
| Final particle number | 88,194 | 97,704 |
| Symmetry imposed | C8 | C9 |
| Global map resolution (Å) | 3.05 | 3.28 |
| (FSC at 0.143, Å) | | |
| EMDB ID | EMD-50061 | EMD-50062 |
| Model building | | |
| Associated PDB ID | 9EYP | 9EYQ |
| FSC map-to-model | 2.98 | 3.20 |
| (0.143)/Å (Å) | | |
| Composition | | |
| Protein chains | 8 | 9 |
| Overall atoms | 12,760 | 13,815 |
| Protein residues | 1,456 | 1629 |
| Ligand molecules | 48 | 45 |
| (sphingomyelin) | | |
| Bonds (R.M.S.D.) | | |
| Length (Å) | 0.012 (0) | 0.012 (0) |
| Angles (°) | 1.817 (49) | 1.788 (36) |
| B-factors (Å²) | | |
| (min/max/mean) | | |
| Protein residues | 30.00/125.37/55.09 | 30.11/125.37/55.23 |
| Ramachandran plot (%) | | |
| Outliers | 0.00 | 0.00 |
| Allowed | 2.78 | 2.23 |
| Favored | 97.22 | 97.77 |
| Rotamer outliers (%) | 0.68 | 0.68 |
| Cβ outliers (%) | 0.00 | 0.00 |
| MolProbity score | 0.64 | 1.09 |
| Clash Score | 0.00 | 2.55 |

### REFERENCES FOR EXAMPLE 2

[1] aM. Marchioretto, M. Podobnik, M. Dalla Serra, G. Anderluh, Biophys. Chem. 2013, 182, 64-70; bA. Crnković, M. Srnko, G. Anderluh, Life 2021, 11, 27.
[2] J. J. Kasianowicz, E. Brandin, D. Branton, D. W. Deamer, Proc. Natl. Acad. Sci. USA 1996, 93, 13770-13773.
[3] M. Jain, S. Koren, K. H. Miga, J. Quick, A. C. Rand, T. A. Sasani, J. R. Tyson, A. D. Beggs, A. T. Dilthey, I. T. Fiddes, S. Malla, H. Marriott, T. Nieto, J. O'Grady, H. E. Olsen, B. S. Pedersen, A. Rhie, H. Richardson, A. R. Quinlan, T. P. Snutch, L. Tee, B. Paten, A. M. Phillippy, J. T. Simpson, N. J. Loman, M. Loose, Nat. Biotechnol. 2018, 36, 338-345.
[4] aJ. A. Alfaro, P. Bohlander, M. Dai, M. Filius, C. J. Howard, X. F. van Kooten, S. Ohayon, A. Pomorski, S. Schmid, A. Aksimentiev, E. V. Anslyn, G. Bedran, C. Cao, M. Chinappi, E. Coyaud, C. Dekker, G. Dittmar, N. Drachman, R. Eelkema, D. Goodlett, S. Hentz, U. Kalathiya, N. L. Kelleher, R. T. Kelly, Z. Kelman, S. H. Kim, B. Kuster, D. Rodriguez-Larrea, S. Lindsay, G. Maglia, E. M. Marcotte, J. P. Marino, C. Masselon, M. Mayer, P. Samaras, K. Sarthak, L. Sepiashvili, D. Stein, M. Wanunu, M. Wilhelm, P. Yin, A. Meller, C. Joo, Nat. Methods 2021, 18, 604-617; bA. Bonini, A. Sauciuc, G. Maglia, Nat. Methods 2024, 21, 16-17; cA. Sauciuc, B. Morozzo Della Rocca, M. J. Tadema, M. Chinappi, G. Maglia, Nature Biotechnol. 2023; dY. L. Ying, Z. L. Hu, S. Zhang, Y. Qing, A. Fragasso, G. Maglia, A. Meller, H. Bayley, C. Dekker, Y. T. Long, Nature Nanotechnol. 2022, 17, 1136-1146.
[5] aG. Huang, K. Willems, M. Bartelds, P. van Dorpe, M. Soskine, G. Maglia, Nano Lett. 2020, 20, 3819-3827; bS. Straathof, G. Di Muccio, M. Yelleswarapu, M. Alzate Banguero, C. Wloka, N. J. van der Heide, M. Chinappi, G. Maglia, ACS Nano 2023, 17, 13685-13699.
[6] D. Rodriguez-Larrea, H. Bayley, Nature Nanotechnol. 2013, 8, 288-295.
[7] S. Zernia, N. J. van der Heide, N. S. Galenkamp, G. Gouridis, G. Maglia, ACS Nano 2020, 14, 2296-2307.
[8] aC. Wloka, V. Van Meervelt, D. van Gelder, N. Danda, N. Jager, C. P. Williams, G. Maglia, ACS Nano 2017, 11, 4387-4394; bS. A. Shorkey, J. Du, R. Pham, E. R. Strieter, M. Chen, ChemBioChem 2021, 22, 2688-2692.
[9] aC. Cao, P. Magalhaes, L. F. Krapp, J. F. Bada Juarez, S. F. Mayer, V. Rukes, A. Chiki, H. A. Lashuel, M. Dal Peraro, ACS Nano 2023, 18, 1504-1515; bL. Restrepo-Perez, C. H. Wong, G. Maglia, C. Dekker, C. Joo, Nano Lett. 2019, 19, 7957-7964; cI. C. Nova, J. Ritmejeris, H. Brinkerhoff, T. J. R. Koenig, J. H. Gundlach, C. Dekker, Nature Biotechnol. 2023.
[10] T. Ensslen, K. Sarthak, A. Aksimentiev, J. C. Behrends, JACS 2022, 144, 16060-16068.
[11] L. Zhang, M. L. Gardner, L. Jayasinghe, M. Jordan, J. Aldana, N. Burns, M. A. Freitas, P. Guo, Biomaterials 2021, 276, 121022.
[12] aH. Ouldali, K. Sarthak, T. Ensslen, F. Piguet, P. Manivet, J. Pelta, J. C. Behrends, A. Aksimentiev, A. Oukhaled, Nat. Biotechnol. 2020, 38, 176-181; bH. Brinkerhoff, A. S. W. Kang, J. Liu, A. Aksimentiev, C. Dekker, Science 2021, 374, 1509-1513.
[13] aJ. Wang, J. D. Prajapati, F. Gao, Y. L. Ying, U. Kleinekathöfer, M. Winterhalter, Y. T. Long, JACS 2022, 144, 15072-15078; bR. C. Abraham Versloot, P. Arias-Orozco, M. J. Tadema, F. L. Rudolfus Lucas, X. Zhao, S. J. Marrink, O. P. Kuipers, G. Maglia, JACS 2023, 145, 18355-18365.
[14] J. K. Aronson, R. E. Ferner, Curr. Protoc. Pharmacol. 2017, 76, 9 23 21-29 23 17.
[15] aJ. Xu, X. Zhang, M. Monestier, N. L. Esmon, C. T. Esmon, J. Immunol. 2011, 187, 2626-2631; bP. Szatmary, W. Huang, D. Criddle, A. Tepikin, R. Sutton, J. Cell. Mol. Med. 2018, 22, 4617-4629; cR. Chen, R. Kang, X. G. Fan, D. Tang, Cell Death Dis. 2014, 5, e1370; dK. Shimono, T. Ito, C. Kamikokuryo, S. Niiyama, S. Yamada, H. Onishi, H. Yoshihara, I. Maruyama, Y. Kakihana, Thrombosis J. 2023, 21, 91.
[16] C. F. Urban, D. Ermert, M. Schmid, U. Abu-Abed, C. Goosmann, W. Nacken, V. Brinkmann, P. R. Jungblut, A. Zychlinsky, PLoS Pathog. 2009, 5, e1000639.
[17] W. Peng, S. Wu, W. Wang, Clin. Exp. Rheumatol. 2023, 41, 1792-1800.
[18] P. Van den Ackerveken, A. Lobbens, D. Pamart, A. Kotronoulas, G. Rommelaere, M. Eccleston, M. Herzog, Sci. Rep. 2023, 13, 16335.
[19] P. Li, S. Liang, L. Wang, X. Guan, J. Wang, P. Gong, Shock 2023, 60, 664-670.
[20] X. Zhan, D. Liu, Y. Dong, Y. Gao, X. Xu, T. Xie, H. Zhou, G. Wang, H. Zhang, P. Wu, X. He, C. Sun, X. Yao, Y. Xu, Adv. Ther. 2022, 39, 1310-1323.
[21] aP. Waduge, R. Hu, P. Bandarkar, H. Yamazaki, B. Cressiot, Q. Zhao, P. C. Whitford, M. Wanunu, ACS Nano 2017, 11, 5706-5716; bJ. Larkin, R. Y. Henley, M. Muthukumar, J. K. Rosenstein, M. Wanunu, Biophys. J. 2014, 106, 696-704.
[22] N. Rojko, M. Dalla Serra, P. Maček, G. Anderluh, Biochim. Biophys. Acta 2016, 1858, 446-456.
[23] aQ. Hong, I. Gutierrez-Aguirre, A. Barlič, P. Malovrh, K. Kristan, Z. Podlesek, P. Maček, D. Turk, J. M. Gonzalez-Manas, J. H. Lakey, G. Anderluh, J. Biol. Chem. 2002, 277, 41916-41924; bK. Kristan, Z. Podlesek, V. Hojnik, I. Gutierrez-Aguirre, G. Gunčar, D. Turk, J. M. Gonzalez-Manas, J. H. Lakey, P. Maček, G. Anderluh, J. Biol. Chem. 2004, 279, 46509-46517; cK. Kristan, G. Viero, P. Maček, M. Dalla Serra, G. Anderluh, FEBS J. 2007, 274, 539-550; dK. Tanaka, J. M. Caaveiro, K. Morante, J. M. Gonzalez-Manas, K. Tsumoto, Nat. Comm. 2015, 6, 6337.
[24] C. Wloka, N. L. Mutter, M. Soskine, G. Maglia, Angew. Chem. Int. Ed. Engl. 2016, 55, 12494-12498.
[25] G. Huang, A. Voet, G. Maglia, Nat. Comm. 2019, 10, 835.
[26] G. Šolinc, M. Srnko, F. Merzel, A. Crnkovič, M. Kozorog, M. Podobnik, G. Anderluh, in press 2024.
[27] aM. Soskine, A. Biesemans, B. Moeyaert, S. Cheley, H. Bayley, G. Maglia, Nano Lett. 2012, 12, 4895-4900; bY. Q. Wang, C. Cao, Y. L. Ying, S. Li, M. B. Wang, J. Huang, Y. T. Long, ACS Sens. 2018, 3, 779-783.
[28] aM. Tejuca, M. D. Serra, M. Ferreras, M. E. Lanio, G. Menestrina, Biochemistry 1996, 35, 14947-14957; bG. Belmonte, C. Pederzolli, P. Maček, G. Menestrina, J. Membr. Biol. 1993, 131, 11-22.
[29] E. M. Nestorovich, T. K. Rostovtseva, S. M. Bezrukov, Biophys. J. 2003, 85, 3718-3729.
[30] S. Yan, L. Wang, X. Du, S. Zhang, S. Wang, J. Cao, J. Zhang, W. Jia, Y. Wang, P. Zhang, H.-Y. Chen, S. Huang, Chem. Sci. 2021, 12, 9339-9346.
[31] M. Y. Li, Y. L. Ying, J. Yu, S. C. Liu, Y. Q. Wang, S. Li, Y. T. Long, JACS Au 2021, 1, 967-976.
[32] aH. Niu, M. Y. Li, Y. L. Ying, Y. T. Long, Chem. Sci. 2022, 13, 2456-2461; bR. C. A. Versloot, S. A. P. Straathof, G. Stouwie, M. J. Tadema, G. Maglia, ACS Nano 2022, 16, 7258-7268.
[33] M. Moiana, F. Aranda, G. de Larranaga, Clin. Biochem. 2021, 94, 12-19.
[34] T. D. Goddard, C. C. Huang, E. C. Meng, E. F. Pettersen, G. S. Couch, J. H. Morris, T. E. Ferrin, Protein Sci. 2018, 27, 14-25.
[35] M. Chinappi, M. Yamaji, R. Kawano, F. Cecconi, ACS Nano 2020, 14, 15816-15828.
[36] Y. Tsunaka, N. Kajimura, S.-i. Tate, K. Morikawa, Nucleic Acids Res. 2005, 33, 3424-3434.
[37] N. F. Lu, L. Jiang, B. Zhu, D. G. Yang, R. Q. Zheng, J. Shao, X. M. Xi, Ann. Palliat. Med. 2020, 9, 1084-1091.
[38] aS. Zhang, Z. Cao, P. Fan, W. Sun, Y. Xiao, P. Zhang, Y. Wang, S. Huang, Angew. Chem. Int. Ed. Engl. 2024, 63, e202316766; bC. Cao, P. Magalhães, L. F. Krapp, J. F. Bada Juarez, S. F. Mayer, V. Rukes, A. Chiki, H. A. Lashuel, M. Dal Peraro, ACS Nano 2024, 18, 1504-1515.
[39] J. Demšar, T. Curk, A. Erjavec, C. Gorup, T. Hočevar, M. Milutinovie, M. Možina, M. Polajnar, M. Toplak, A. Starič, M. Štajdohar, L. Umek, L. Žagar, J. Žnbontar, M. Žitnik, B. Zupan, J. Mach. Learn. Res. 2013, 14, 2349-2353.
[40] S. K. Maheshwaram, D. Shet, S. R. David, M. B. Lakshminarayana, G. V. Soni, ACS Sensors 2022, 7, 3876-3884.
[41] S. E. Van der Verren, N. Van Gerven, W. Jonckheere, R. Hambley, P. Singh, J. Kilgour, M. Jordan, E. J. Wallace, L. Jayasinghe, H. Remaut, Nat. Biotechnol. 2020, 38, 1415-1420.
[42] Z. Ji, M. Jordan, L. Jayasinghe, P. Guo, Nanomed. Nanotechnol. Biol. Med. 2020, 25, 102170.
[43] M. Podobnik, P. Savory, N. Rojko, M. Kisovec, N. Wood, R. Hambley, J. Pugh, E. J. Wallace, L. McNeill, M. Bruce, I. Liko, T. M. Allison, S. Mehmood, N. Yilmaz, T. Kobayashi, R. J. Gilbert, C. V. Robinson, L. Jayasinghe, G. Anderluh, Nat. Comm. 2016, 7, 11598.
[44] A. Punjani, J. L. Rubinstein, D. J. Fleet, M. A. Brubaker, Nat. Methods 2017, 14, 290-296.
[45] P. Emsley, K. Cowtan, Acta Cryst. Section D 2004, 60, 2126-2132.
[46] T. Croll, Acta Cryst. Section D 2018, 74, 519-530.
[47] T. D. Goddard, C. C. Huang, E. C. Meng, E. F. Pettersen, G. S. Couch, J. H. Morris, T. E. Ferrin, Protein Sci. 2018, 27, 14-25.
[48] D. Liebschner, P. V. Afonine, M. L. Baker, G. Bunkóczi, V. B. Chen, T. I. Croll, B. Hintze, L. W. Hung, S. Jain, A. J. McCoy, N. W. Moriarty, R. D. Oeffner, B. K. Poon, M. G. Prisant, R. J. Read, J. S. Richardson, D. C. Richardson, M. D. Sammito, O. V. Sobolev, D. H. Stockwell, T. C. Terwilliger, A. G. Urzhumtsev, L. L. Videau, C. J. Williams, P. D. Adams, Acta Cryst. Section D 2019, 75, 861-877.
[49] E. Krissinel, K. Henrick, Acta Cryst. Section D 2004, 60, 2256-2268.

### Example 3

Lipid membranes are crucial for the functioning of organisms. A significant portion of the mammalian proteome is associated with lipid membranes¹ and many membrane-associated proteins are valuable drug targets². It is becoming increasingly clear that lipids can affect proteins in many different ways, especially when regulating the structure and function of membrane proteins³⁻⁷. For example, the membrane lipids can also act as cofactors required for the proper functioning of membrane enzymes⁸ or as structural support in formation of membrane protein assemblies⁹. Lipids, directly and indirectly, contribute to ion channel gating and activity by directly interacting with the protein or altering the properties of the membrane¹⁰. They play a significant role in regulating the activity of G-protein-coupled receptors (GPCRs), such as serotonin receptors¹¹ and β2-adrenergic receptors¹², via specific binding of phospholipids or by cholesterol molecules surrounding the receptor's transmembrane domain influencing the ligand-binding pocket.

Due to the nature of protein-lipid interactions and the molecular dynamics of lipid molecules, the determination of protein-lipid interactions at high resolution is a major challenge^{13,14}. Thanks to advances in cryo-electron microscopy (cryo-EM) an ever-increasing number of structures of membrane proteins with associated lipids are known^{14,15}. The lipids observed in these structures are usually one of two types, a single, specifically bound lipid with a well-defined density or multiple ordered aliphatic chains without defined headgroups in a cleft or between interfaces between subunits of a larger complex^{16,17}. In several cases lipids have been unambiguously identified in three dimensional structures of membrane protein complexes, providing valuable information about their effects on protein structure and function^{11,18-20}.

The crystal structure of the octameric transmembrane pore of the pore forming toxin, an actinoporin fragaceatoxin C (FraC)²¹ shows the positions of the headgroups of three lipids bound to a single protomer. However, how the membrane is organized around the pore and how the pore itself affects the arrangement of membrane lipids, has not been yet shown in detail. Here we have used a homologue of FraC from the coral Orbicella faveolata, Fav, pore to specifically identify and reconstruct the atomic model of lipid molecules associated with the membrane-inserted pore using cryo-electron microscopy (cryo-EM). We have developed a protocol to produce stable soluble pores retaining a significant proportion of lipids that we resolved with sufficiently high resolution to assign different functional roles. In combination with molecular dynamics (MD) simulations, we reveal extensive lipid-protein interactions and provide unprecedented insight into the embedding of the protein complex in the lipid membrane.

### Results

### Preparation of Soluble Fav Pores

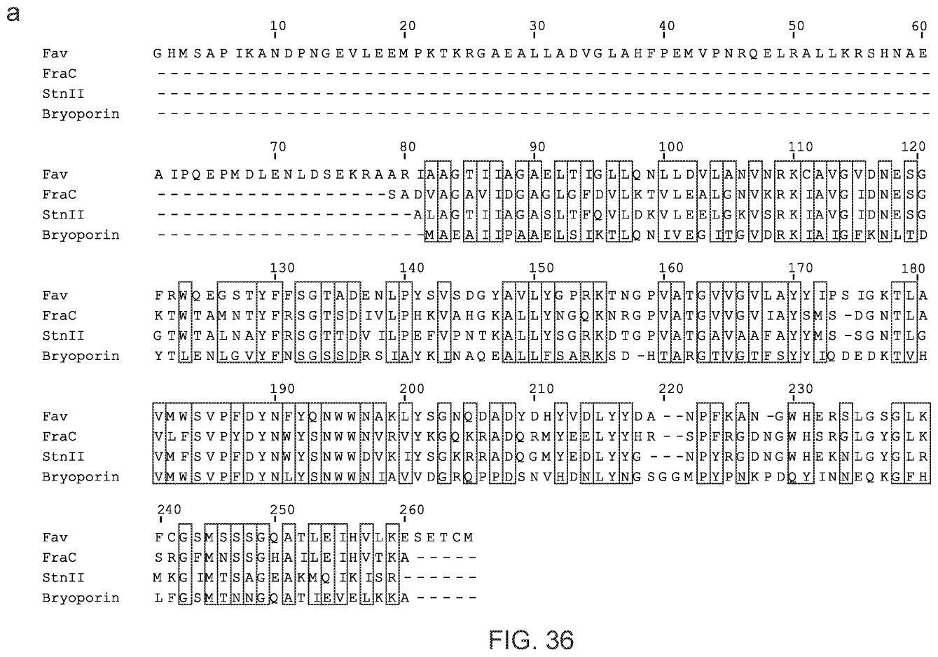

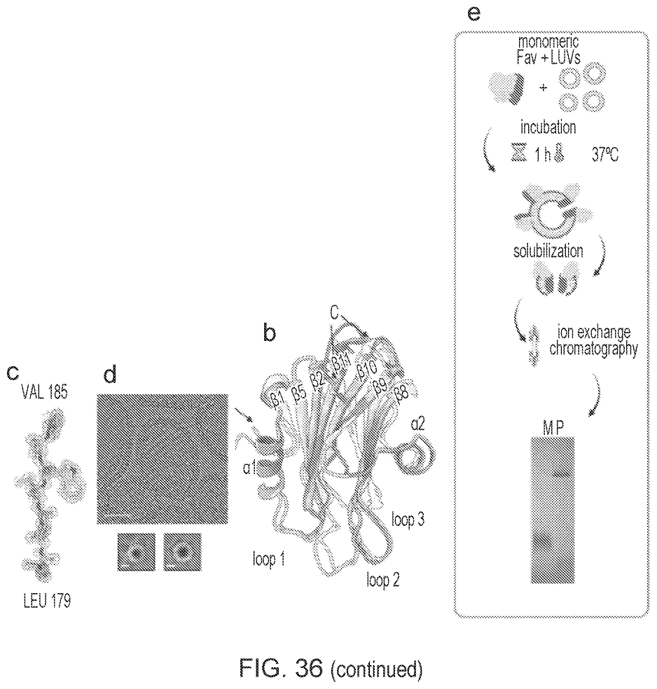

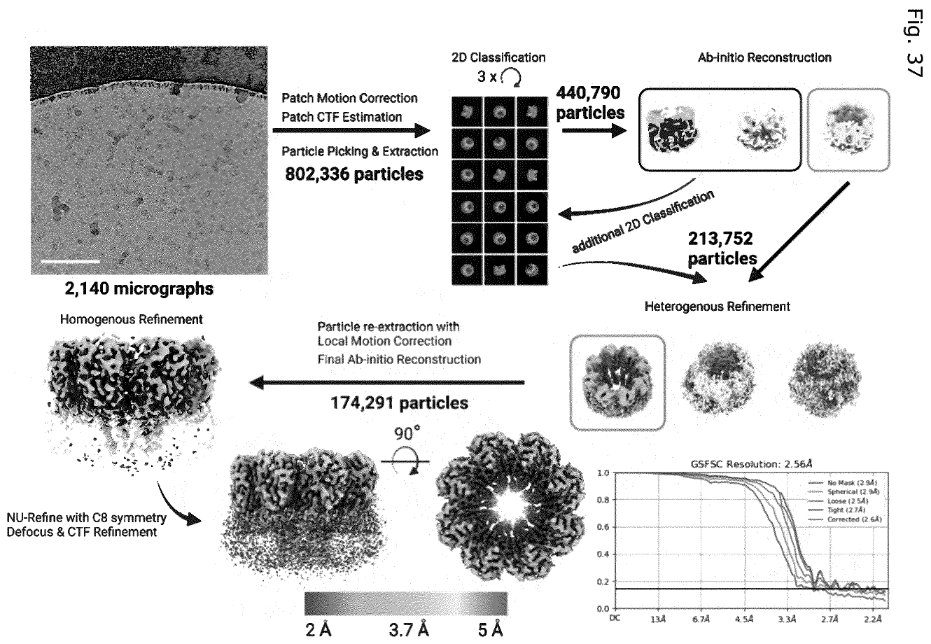

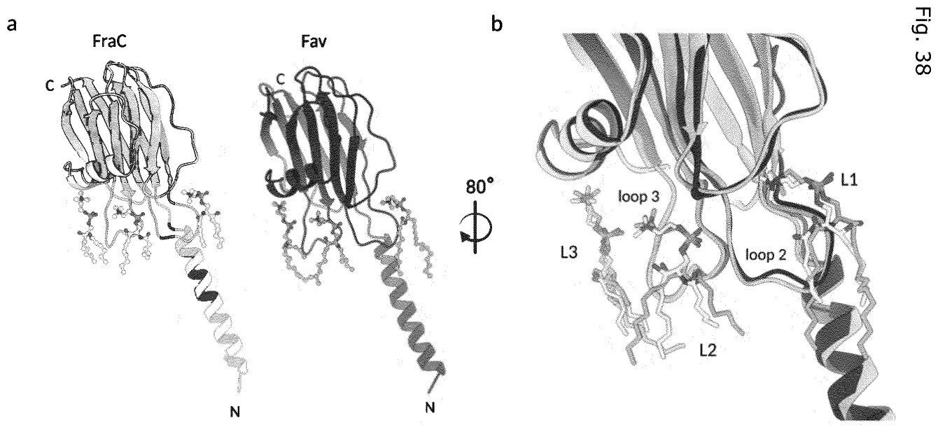

Fav is a homologue of actinoporins with unique extensions at the N and C termini (FIG. 36a). We prepared a protein construct with removed 53 residues at the N-terminus (ΔN53Fav) as this region was predicted by Swiss Model²² to be unstructured. The X-ray crystal structure of the ΔN53Fav at 1.5 Å resolution showed a clear conservation of a typical actinoporin monomer consisting of a central β-sandwich flanked by α-helices (FIGS. 36b and c, Table 2)²³. While there was no defined electron density for the residues up to Ala 79, the C-terminal Fav extension was clearly resolved and anchored to the β-sandwich by a disulphide bond (FIG. 36b). For cryo-EM analysis, Fav pores were prepared by incubating monomeric wild type Fav with 1,2-dioleoyl-sn-glycero-3-phosphocholine (DOPC):sphingomyelin (SM) 1:1 (mol:mol) large unilamellar vesicles (FIG. 36c). We clearly observed two stoichiometries, octameric and nonameric ones (FIG. 36c). We tested different detergents to solubilize the pores and finally solubilize them with lauryl dimethylamine oxide. We separated the solubilized pores from excess lipids and detergents by ion exchange chromatography and finally concentrated them (FIG. 36d).

### Cryo-EM Structure of Solubilized Fav Pores Reveals a Layer of Lipids Supporting the Pore Structure

3D cryo-EM reconstruction at 2.6 Å of solubilized pores (FIG. 37) revealed a funnel-shaped pore consisting of eight identical subunits (FIG. 33a). We did not observe any nonameric pores, indicating that they were not stable enough to resist solubilization by detergents or sample concentration step before vitrification. The transmembrane channel, composed of a cluster of eight amphipathic α-helices, was embedded in a micelle composed of detergents and remnant lipids. Eighty amino acid residues preceding the α-helix were not defined by the cryo-EM density due to their flexibility (FIG. 33a). While the overall architecture of the pore, including the cap region and the arrangement of the transmembrane helical cluster (FIG. 33b) is similar to that of FraC²¹, there are the following notable differences. In contrast to the FraC pore, the electrostatic surface potential of the pore is predominantly negative (FIG. 33c). The constriction of the channel is formed by the cluster of E91 side chains, which form the narrowest part of the channel with a diameter of 1.55 nm (FIG. 33c). Finally, the elongation at the C-terminus of Fav provides additional contacts between the protomers (FIG. 33c).

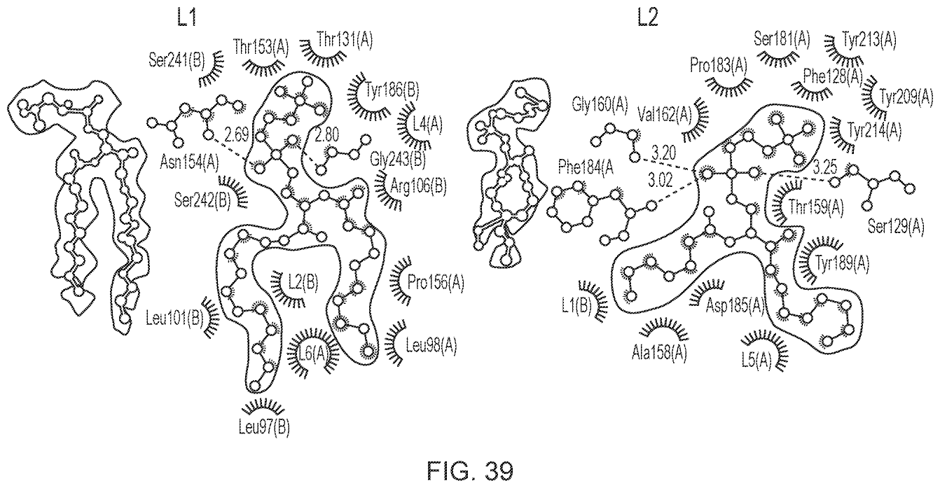

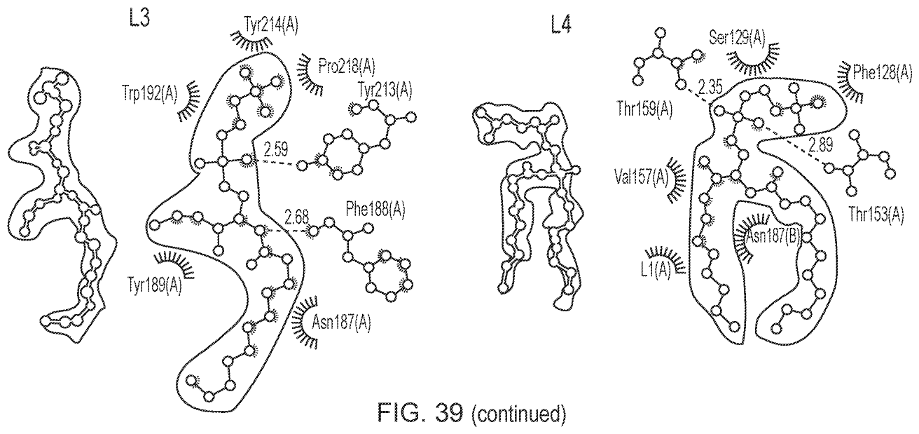

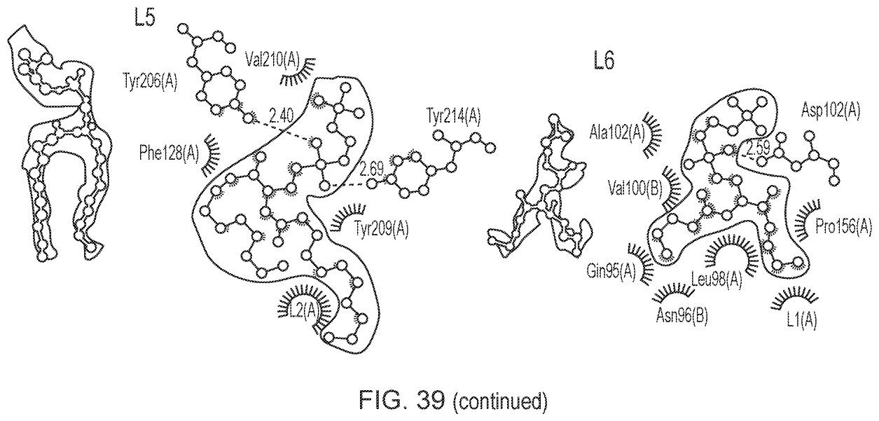

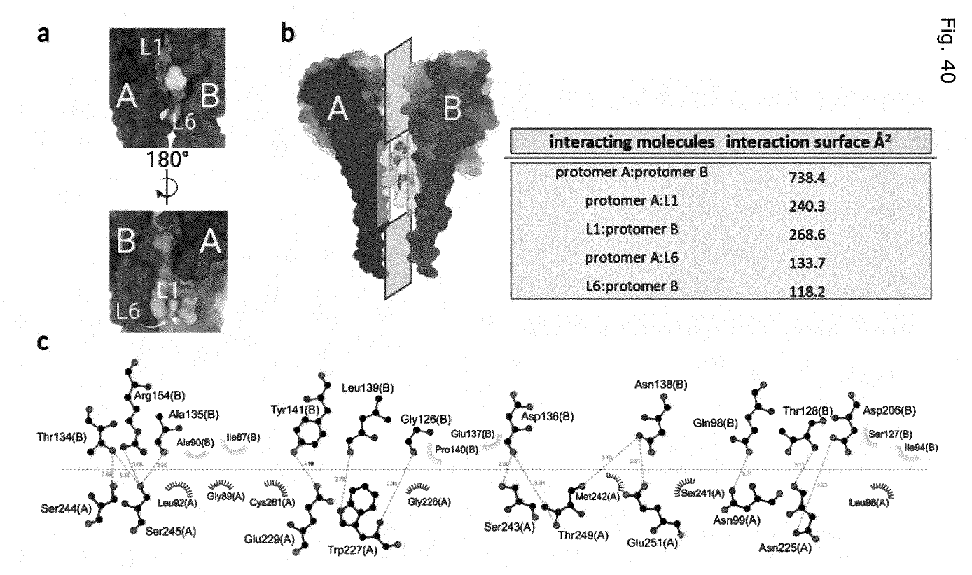

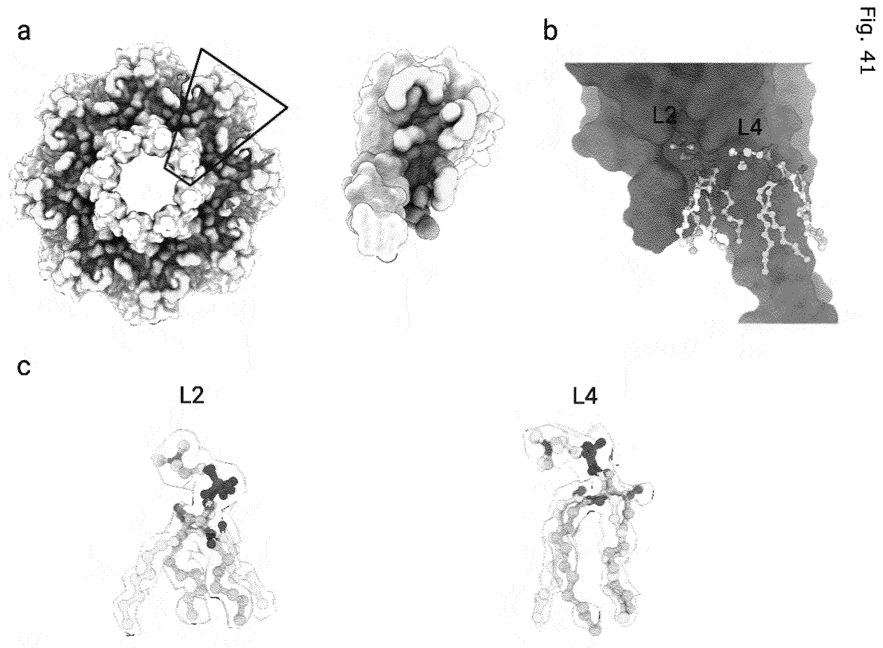

Three lipid molecules per protomer with partially resolved acyl chains were previously observed in the FraC pore (FIG. 38)²¹. In the Fav pore, we clearly observed lipid densities at the corresponding positions, labelled L1-L3 (FIG. 33d, FIG. 38). L1 is located between two neighbouring protomers. The head group of L1 is almost completely buried inside the complex and forms hydrophobic interactions as well as hydrogen bonds via main chain amine groups of N154 of one protomer and G243 of the other (FIG. 39). L2 is located below the C-terminal helix and between loops 2 and 3 and L3 is located above loop 3 (FIG. 33d, FIG. 39). In addition to these three, we observed densities corresponding to three other lipids in the Fav pore, L4-L6. L4 is located to the side of loop 2 towards the inner pocket between two protomers, forming two hydrogen bonds with side chains of T153 and T159. L5 is located at the outer edge of the pore and interacts with the C-terminal α-helix, almost completely covering L2 (FIG. 33d). It forms two hydrogen bonds with side chains of tyrosines Y206 and Y214 from the α-helix (FIG. 39). L6 is located at the edge of the fenestration, with the head group pointing towards the pore lumen. L6 forms hydrophobic interactions with two protomers and a hydrogen bond with the side chain of D102 (FIG. 33d, FIG. 39).

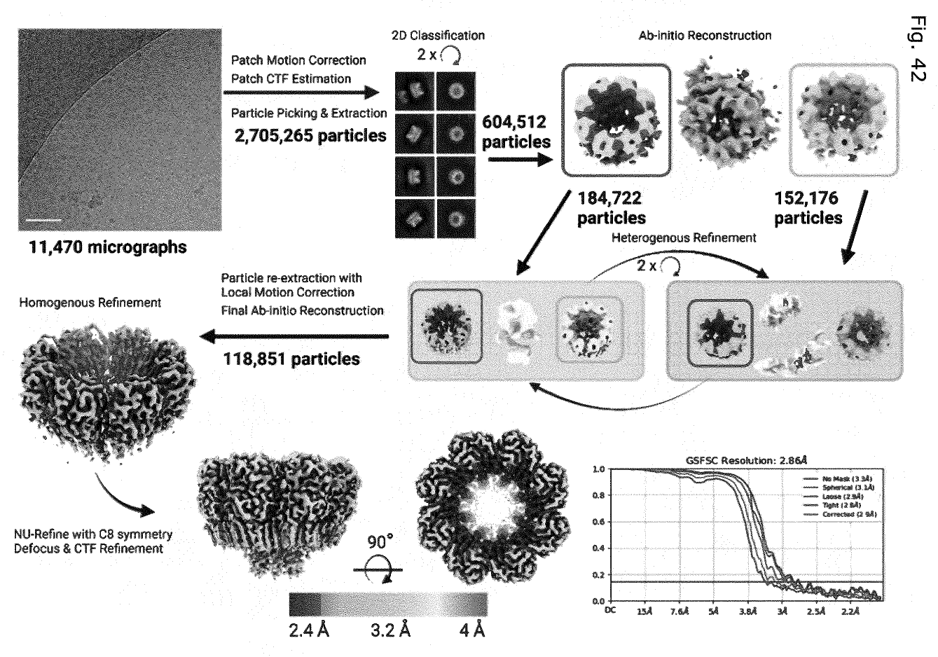

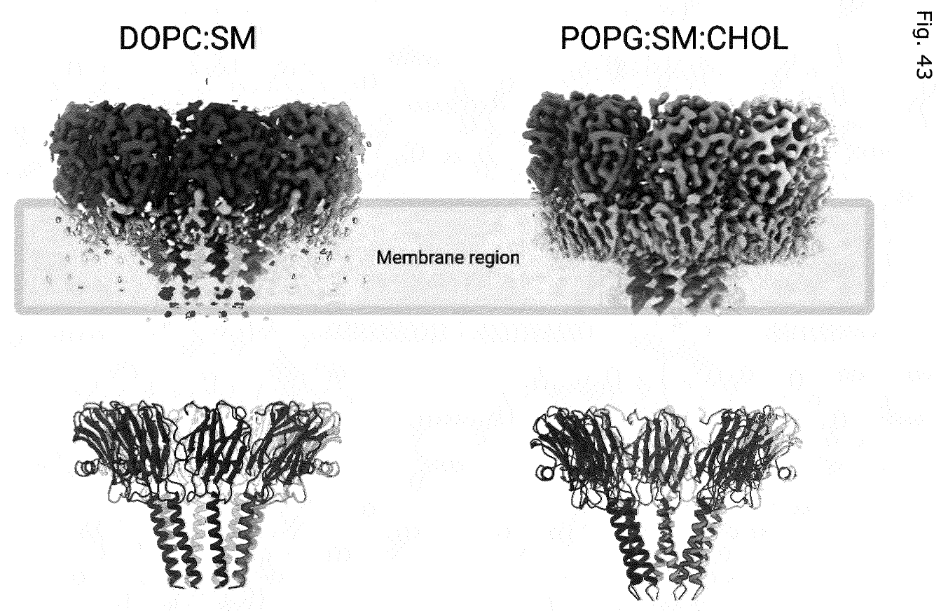

Altogether, the isolated wild-type pore of Fav is a protein-lipid pore complex that consists of 8 homoprotomers to which 48 lipids were found stably bound. The lipids are bound to the protein via electrostatic interactions and there are also extensive hydrophobic interactions between the lipids (FIG. 39). L1 and L6 form extensive contacts between the protomers and are therefore referred to as structural lipids (FIG. 40). The headgroups of L1-L5 are well defined (FIG. 39) and correspond to the shape of the phosphocholine headgroups of DOPC or SM used in vesicles. The densities corresponding to the aliphatic chains vary between 4 (i.e. in L6) and 12 methyl groups (i.e. in L1). The region under the cap of the pore is empty (FIG. 41a). In this void, we could observe alternative conformations of the aliphatic chains of L2 and L4 (FIGS. 41b and c).

### Cholesterol Nanodomains Form Under the Pore

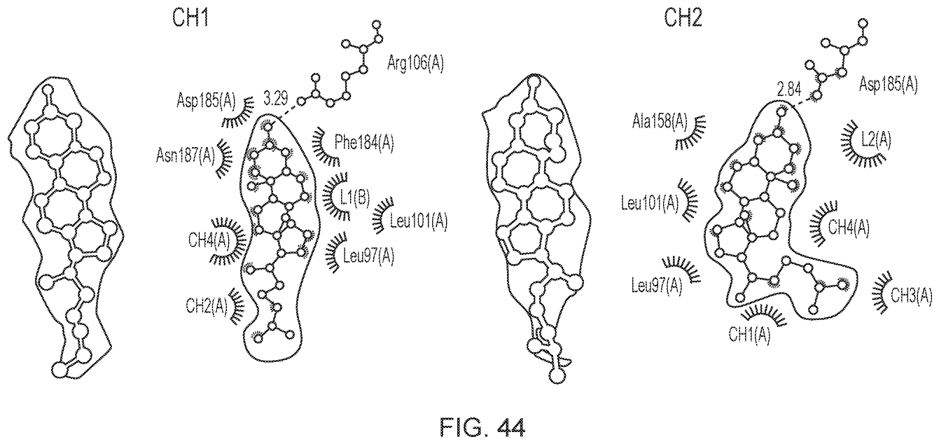

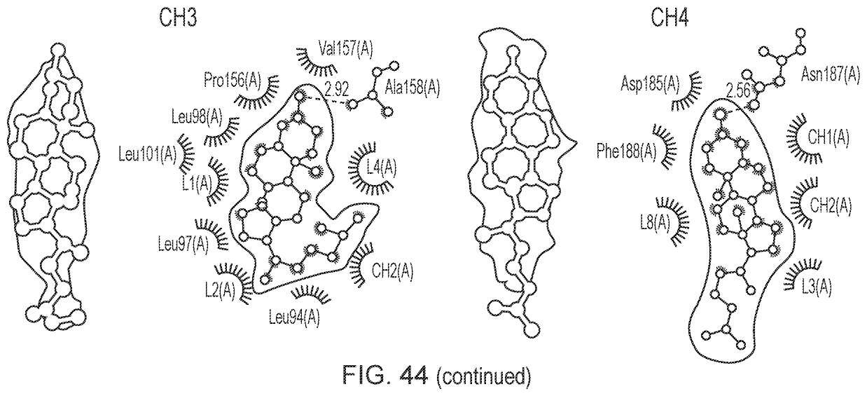

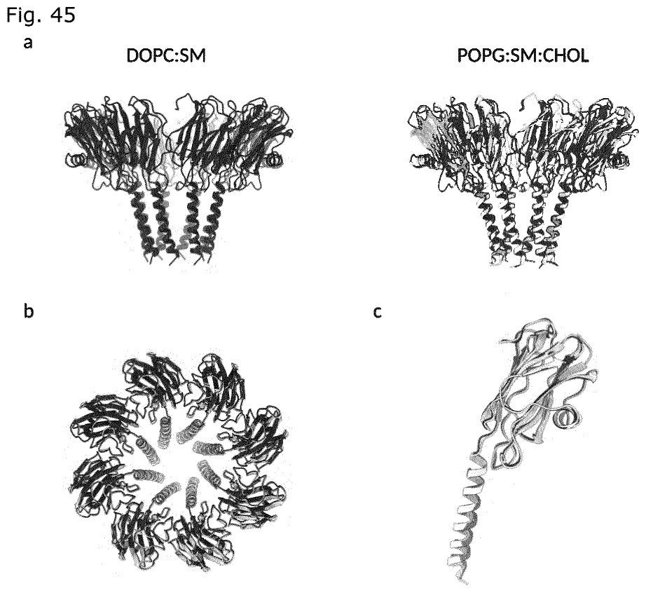

Actinoporin activity is enhanced in its presence of cholesterol²⁴, therefore, our next goal was to determine how cholesterol affects the pore structure. Furthermore, we could not resolve which lipid is bound in the pores formed on DOPC:SM membranes, as the DOPC and SM headgroups have the same phosphocholine structure. For this reason, we used 1-palmitoyl-2-oleoyl-sn-glycero-3-phospho-(1′-rac-glycerol) (POPG) instead of DOPC, which has a different head group than SM and would allow differentiation of the bound lipids. We prepared wild-type Fav pores on liposomes composed of POPG:SM:cholesterol 1:1:1 (mol:mol:mol). Cryo-EM reconstruction at 2.8 Å resolution showed a similar architecture of the pore as found in DOPC:SM membranes described above (FIG. 37a, FIG. 42). However, the lipid region below the pore cap was much larger and better defined (FIG. 43). Directly adjacent to the transmembrane helices and below the membrane binding loops 2 and 3 is cholesterol nanodomain composed of four cholesterol molecules per protomer. Each cholesterol molecule is bound to the protomer via a single hydrogen bond (FIG. 37b, FIG. 44). These four cholesterol molecules, with a total van der Waals volume of 1311 Å³, fill the void under the cap of the pore and allow a tighter arrangement of the aliphatic chains of other lipids bound to the pore. The presence of cholesterol in the membrane does not affect the position of the transmembrane helices (FIG. 45). However, it has a small but significant effect on the ionic currents flowing through the pore. The increase in current noise upon pore formation was lower in cholesterol-containing membranes compared to cholesterol-free membranes (FIGS. 37c and d).

Cholesterol Stabilizes Lipids in Contact with the Pore

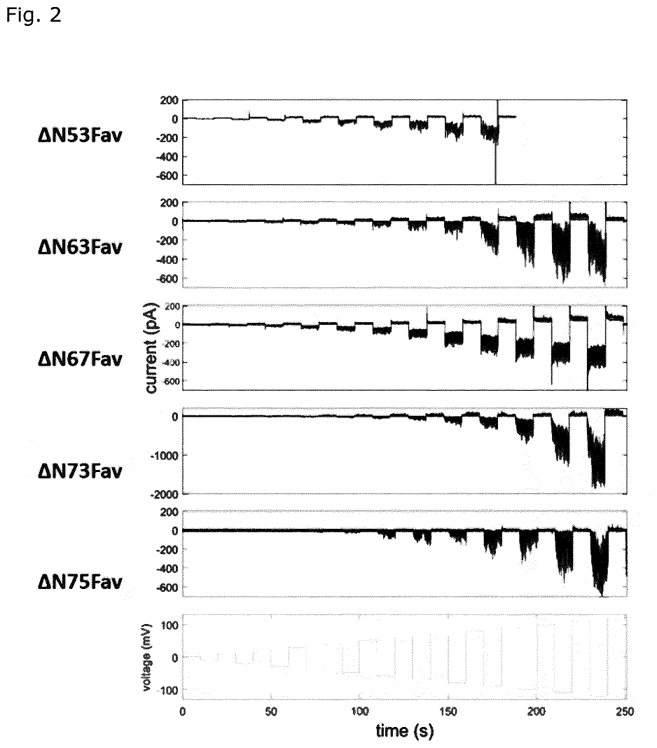

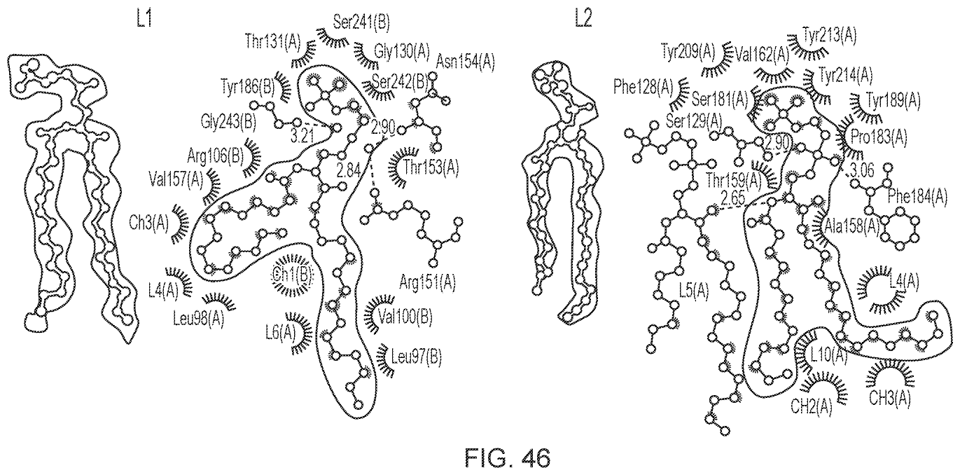

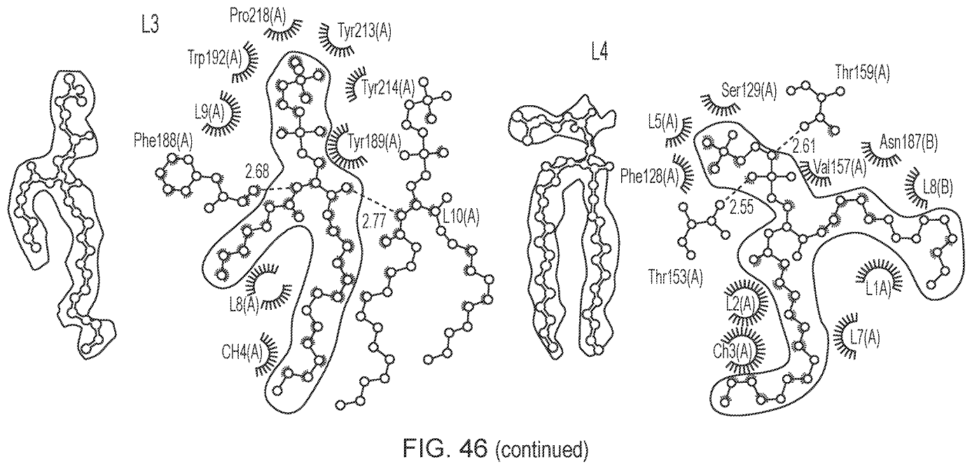

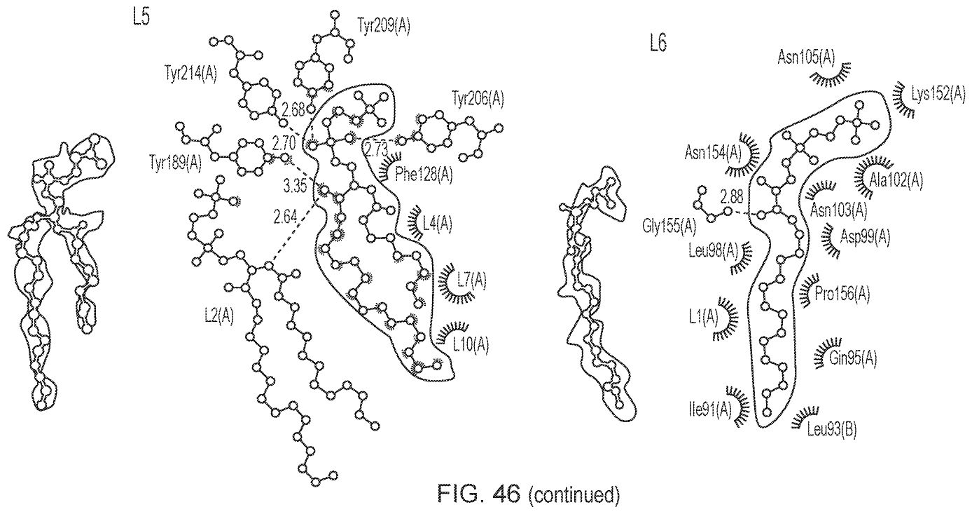

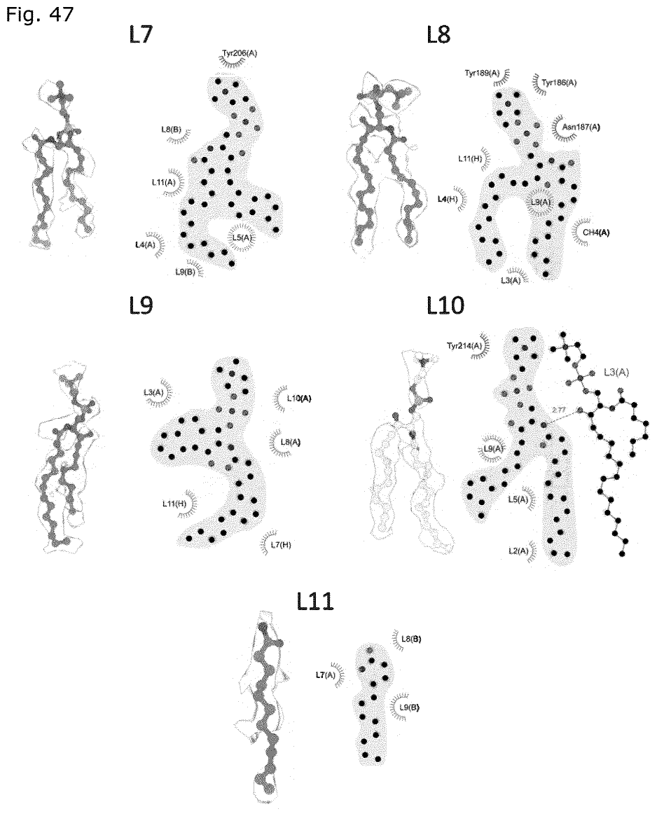

In the presence of cholesterol, we were able to place L1-L6 into the cryo-EM density at the analogous positions as in the pores formed on DOPC:SM membranes (FIG. 46). Moreover, we were able to build four additional phospholipids per protomer and an additional acyl chain (FIGS. 37e and 2f, FIG. 47). Three of these, L7-L9, are located between two protomers (FIG. 37f), however, with very few contacts with them. L7 is located on one side of the protomer, and L8 and L9 are on the other side (FIGS. 37f and g). L7 interacts only with Y206 and is additionally stabilised by hydrophobic interactions with the surrounding lipids L4, L5 and L11 of the same protomer and with L8 and L9 of the neighbouring protomer (FIGS. 37f and g, FIG. 47). The head group of L8 interacts with residues Y186, N187 and Y189 of loop 3 and aliphatic chains of L8 are surrounded by one of the cholesterol molecules (CH4), and lipids L3, L4, L9 and L11 (FIG. 47). Similar to L7, L9 also has a very limited interaction surface with the protomer and is mainly stabilised by lipid-lipid interactions involving L3, L8 and L10 from one protomer and L7 and L11 from the other protomer (FIG. 47, Table 3). We have termed L7-L9 as bridging lipids because these lipids have very little contact with protomers, are in contact with each other and, therefore, interactions between the protomers are indirectly strengthened via a protomer-1-lipid-lipid-protomer-2 bridging (FIG. 37g). We can also observe an additional phospholipid L10 at the outer edge of the lipid binding region (FIG. 37g). This lipid has very little contact with the protein involving Y214 (FIG. 47, Table 3), but forms a hydrogen bond with L3. Finally, we observed an additional individual acyl chain of lipid L11 at the outer edge of the pore, which is stabilized by hydrophobic interactions involving L7 from one protomer and L8 and L9 from the other one (FIG. 47).

Pores prepared using POPG:SM:cholesterol membranes, in combination with the good local resolution of the map, allowed us to identify lipids L1-L5 as SM. The map quality of the headgroup region of L6-L11 is not high enough to distinguish between POPG and SM headgroups. In the presence of cholesterol, acyl chains of lipids are much better defined and are now for most of the observed lipids longer than 10 methyl groups, reaching 15 methyl groups in L1, L2 and L5. Of the total available 9,339 Å² surface area of the single protomer in the pore, the amphipathic transmembrane helix accounts for about 2,000 Å². Of the remaining surface area, 1,500 Å² is accounted for by protomer-protomer interactions, which is comparable to the 2,106 Å² used for interactions with lipid headgroups (Table 3). The extensive surface of the protomer is therefore suited and used for lipid binding. In summary, we could resolve 15 lipids binding each protomer in the Fav pore, which in total includes 32 cholesterol molecules and 80 phospholipids and 8 acyl chains. This monolayer patch is fixed by a network of predominately hydrophobic interactions as well as hydrogen bonds. Twelve hydrogen bonds are formed between protein and lipids and two are formed between lipids (between L2-L5 and L3-10) (FIG. 37h).

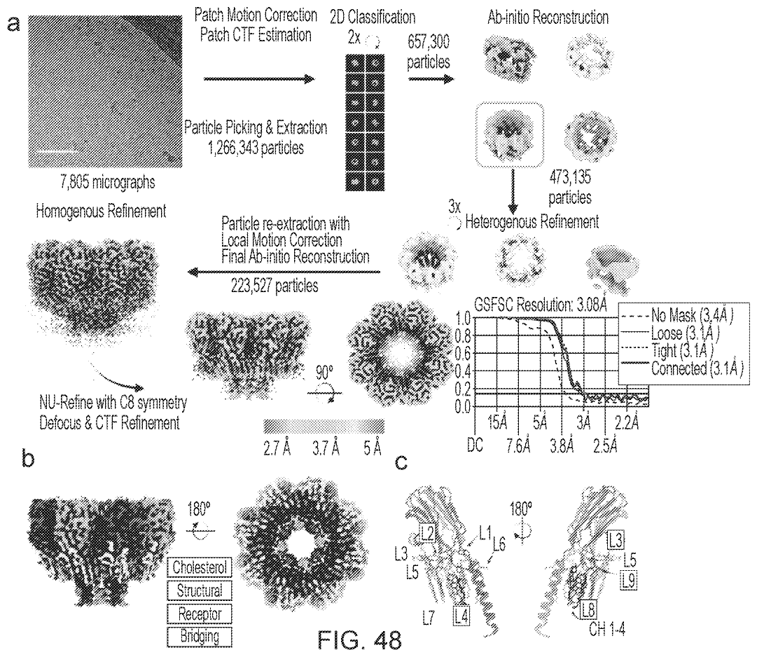

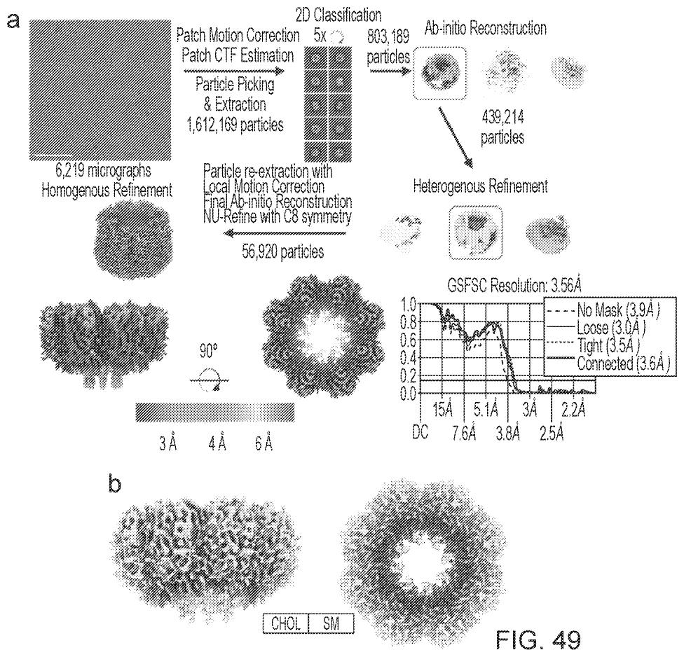

To confirm that cholesterol and not POPG is responsible for tighter lipid arrangement we solved the structure of solubilized octameric pore prepared on DOPC:SM:CHOL vesicles to a resolution of 3.1 A (FIG. 48a). The overall structure is nearly identical with lipids visible at comparable positions (FIGS. 48b and c). In addition, we prepared pores on lipid nanodiscs to exclude the possibility that the observed lipid positions are an artefact of pore solubilization with detergents. Nanodiscs composed of DOPC:SM:CHOL (1:1:1) were incubated with monomeric wild type Fav and then analysed with cryo-EM. We could resolved the octameric pore structure with an overall resolution of 3.6 Å (FIG. 49a). Although the quality of the map is lower compared to that of solubilized pores, we can observe non-proteinaceous densities in the regions corresponding to the positions of phospholipids L1-5 and all four cholesterol molecules (FIG. 49b) further proving that cholesterol binding positions found in Fav pores are real.

### Conformation of Loops 2 and 3 is the Same Between the Soluble Monomer and the Pore Protomer

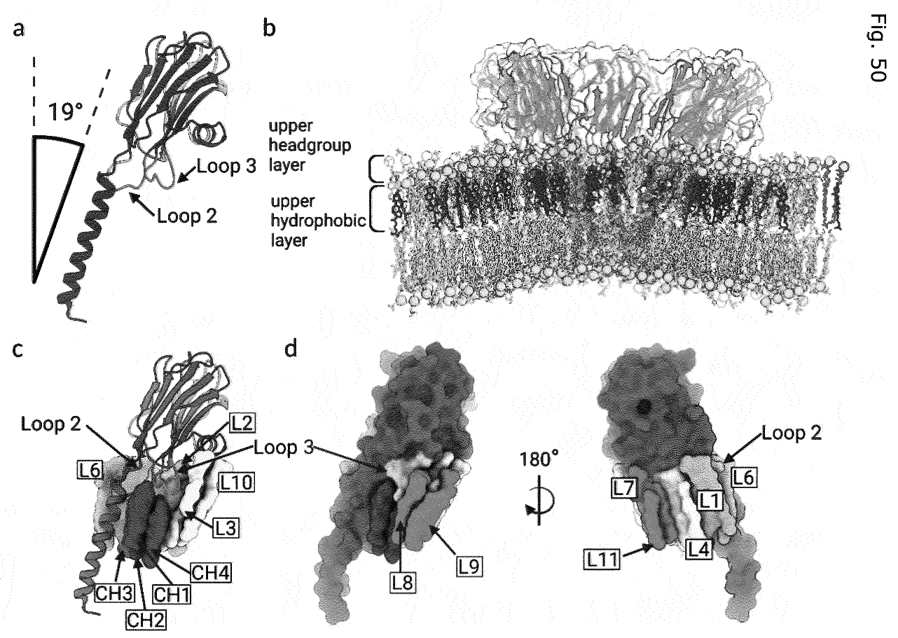

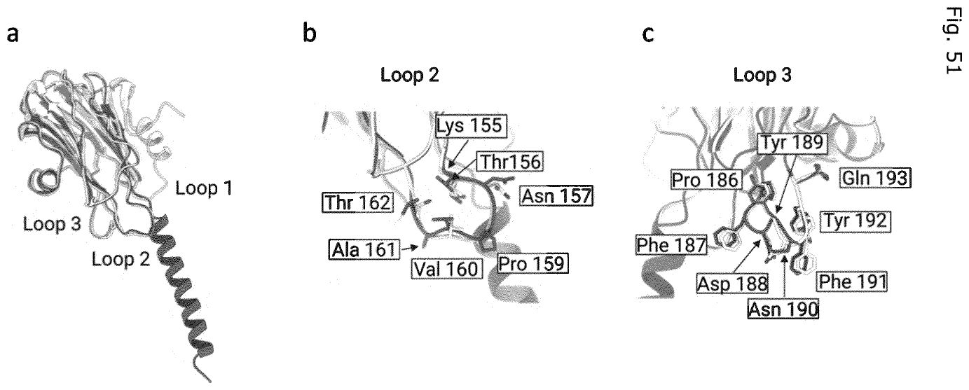

Once the pore is formed, each protomer span the membrane at a 190 angle (FIG. 50a). The amphipathic N-terminal helix passes through the membrane with the hydrophobic side orientated towards the membrane across its entire length. Loops 2 and 3 are immersed in the phospholipid headgroup layer of the membrane and sit on the hydrophobic layer where cholesterol and aliphatic chains are located (FIG. 50b). The cholesterol cluster is packed under loops 2 and 3, while the external side of the loops is wrapped by L1, L2, L3, L4, L6 and L8 (FIG. 50c, d). Surprisingly, the structure of the two loops remained practically unchanged during the transition from the monomer in solution to the membrane-bound protomer, including the main chain and the side chain atoms of the residues involved in the lipid binding (FIG. 51).

### Sphingomyelin is Essential for Oligomerization of Fav

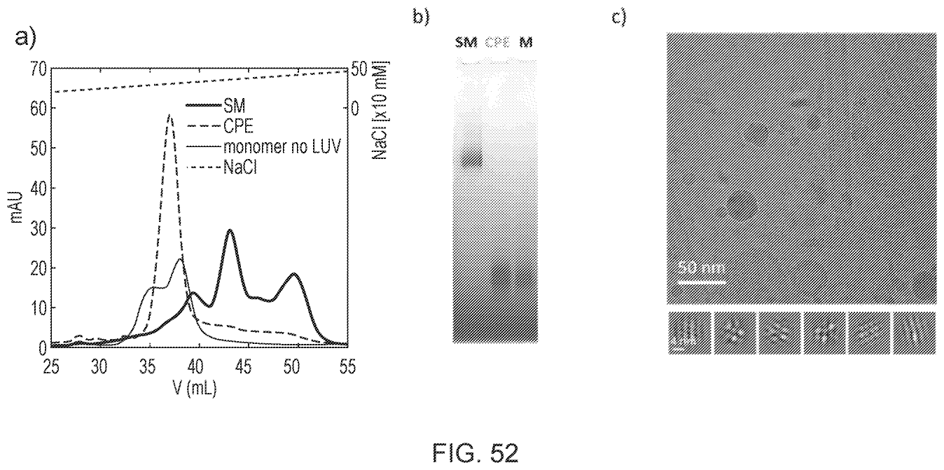

It is known that actinoporins require SM^{25,26} to effectively bind to the membrane and form pores. Our pore structures showed that lipids at two positions play an important structural role. Actinoporins sticholysin II and equinatoxin II, and homologue bryoporin can bind to ceramide phosphoethanolamine (CPE), which has a much smaller ethanolamine headgroup, but their permeabilization activity in CPE-containing membranes is much lower compared to SM-containing membranes^{27,28}. Using our established method for pore formation and purification (FIG. 36d), we were unable to obtain pores from vesicles composed of DOPC:CPE:CHOL in a 1:1:1 (mol:mol:mol) ratio (FIG. 52a, b). Cryo-EM micrographs of DOPC:CPE:CHOL 1:1:1 multilamellar vesicles incubated with monomeric Fav showed that the membranes were completely covered with the protein, but no pores were observed. 2D class averaging showed tightly packed protein particles that correspond in size to a monomeric form of Fav, and bound to the membranes in multiple rows (FIG. 52c). CPE thus enables the binding of protein to the membranes, but is not able to trigger oligomerisation.

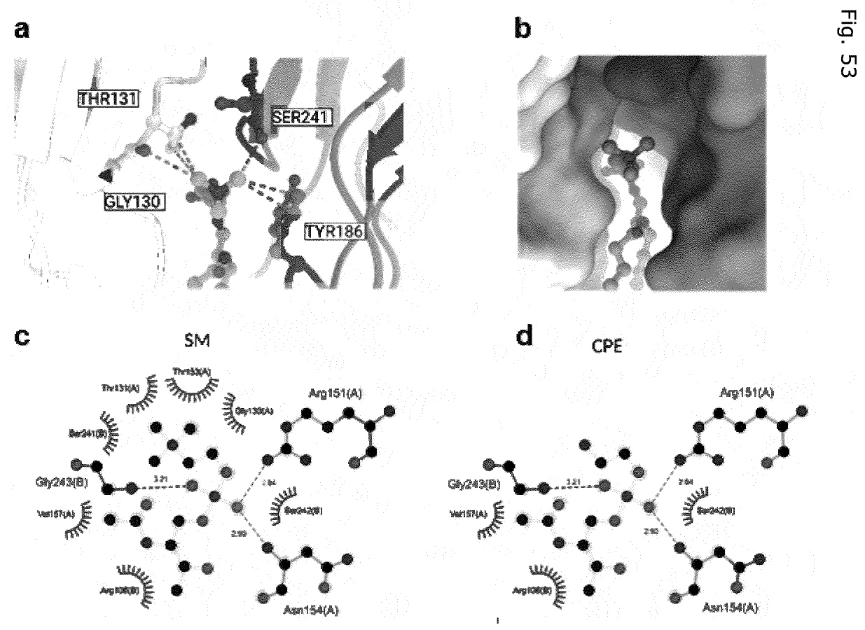

Due to its position in the pore structure, L1 is probably the key lipid responsible for oligomerisation. The L1 head group is coordinated by ten residues, six by one and four by the neighbouring protomer. R151 and N154 of one protomer and G243 of the adjacent protomer can form hydrogen bonds with the phosphate group, while G130, T131, T153 and V157 of one protomer and R106, S241 and S242 of the adjacent protomer coordinate the headgroup with hydrophobic interactions (FIGS. 53a and b). When CPE is modelled at the position of L1, it loses the hydrophobic interactions with four residues (FIG. 53d), which in the case of SM stabilise the protomers in an oligomerised form (FIG. 53b).

### Specifically Bound Lipids Increase the Effect Fav Pore has on Bulk Lipids

All-atom molecular dynamics (MD) simulations of the protein pore in a POPC:SM:CHOL membrane with a molar ratio of 1:1:1 were used to verify bound lipid molecules and clarify the stabilizing role of the protein pore for the surrounding lipid molecules. During the 1.2 μs MD simulation, starting from the single pore structure with 80 SM and 32 cholesterol molecules in their predefined binding positions, not a single one of these predefined lipid molecules was detected to leave its original position.

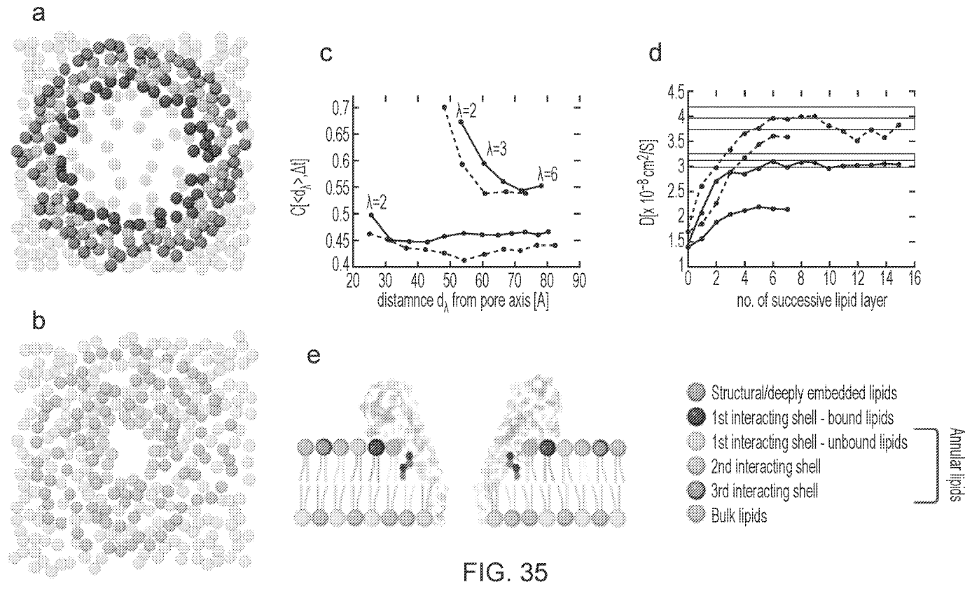

To elucidate the effect of the pore-lipid interactions on structural and dynamic features of the pore surrounding lipid molecules we focus on the correlation between displacements of lipid molecules as well as the lateral lipid diffusion as a function of the distance from the pore. To capture enough details, we grouped SM and POPC lipid molecules into successive coordination shells around the pore in the upper and lower leaflet of the membrane as indicated in FIGS. 35a and b. According to the evaluated correlation, the relative motion of lipid molecules is significantly more correlated in the upper leaflet as compared to the lower one (FIG. 35c). The first lipid shells (λ=1) in both leaflets correspond to lipid molecules in direct contact with the pore, specifically in the upper leaflet these are limited to phospholipid molecules on the external ring of predetermined lipids involving L2, L3, L5, L7, L8, L9 and L10. The subsequent shells (λ=2, 3 . . . ) already refer to molecules interacting exclusively with lipids. As a consequence of the higher correlation driven by the strong lipid-protein cap interaction, the diffusion in the upper leaflet is significantly slower compared to the lower leaflet as shown in FIG. 35d.

### Conclusions

The cryo-EM structures of the Fav pores reveal general principles of protein interaction with lipids, such as details of interaction with cholesterol, with head groups of sphingolipids, how protein loops are immersed in the lipid bilayer, and together with MD simulations also highlight the concept of annular lipids that extend further than the first interactive shell of lipids. Based on the structural and functional data, we can assign different functional roles to the lipids (FIG. 35e). The structural lipids L1 and L6 are integral part of the oligomeric structure. They provide an important interaction surface between the protomers and are thus an essential structural component of the pore. L1 is also required for the oligomerization of the protein protomers into a stable octameric membrane complex. One protomer binds four receptor lipids, (L2-L5), which were confirmed at the high-resolution level to be SM molecules, for which the protein surface is very well suited. The structures also show a cholesterol nanodomain that fits very well into the gap created under the cap of the Fav pore that partially sinks into the membrane via the insertion of loops 2 and 3 of each Fav protomer in the vicinity of the transmembrane helix. We provide high-resolution evidence that the Fav surface can uniquely bind multiple lipid headgroups, challenging the conventional notion of a single lipid acceptor for peripheral membrane proteins^{29,30}. The protein is immersed in the lipid membrane as a rigid object. The structure of the membrane binding region of the protein including the loops 2 and 3 that sink into the membrane does not change during the transition from monomer to pore. This means that the surface is already perfectly adapted for membrane binding and insertion in proteins' water-soluble form. The binding of multiple lipid molecules anchors the proteins more stably to the membranes. The bridging lipids have very little contact with the protein and can provide additional stability for pore assembly through extensive lipid-lipid interactions. This is highlighted by the fact that the presence of cholesterol allows for stable soluble nonameric pores with roughly half of pore particles belonging to nonameric 3D class (FIG. 42), which were not stable in solubilized state when pores were prepared on membranes without cholesterol (FIG. 36). Most of the bound lipid molecules exhibit low mobility in MD simulations, suggesting that the cap region of the pore almost completely restricts lipid movement below the cap and that the pore sits on a lipid membrane patch consisting of at least 112 lipid molecules all of which stayed at their predefined position during 1.2 μs of the simulation.

The pore structure also makes it possible to study the diffusion of lipids in two separate monolayers. In the upper layer, the phospholipid molecules interact with the bridging lipids, while in the lower monolayer the interactions take place with the cluster of transmembrane helices. The movement of lipids estimated from atomistic simulations is correlated with the movement of the pore based on the distance to it. Interestingly, the correlation of the movement between lipids falls off differently in the two leaflets. In the bottom leaflet where we observed no specific lipid binding sites on the α-helices, the lipid-lipid correlation falls off completely after lipid interaction shell 2, whereas the lipid-lipid correlation remains significant for shells 3 and even 4 in the upper leaflet. This excludes the predefined lipids we observe with cryo-EM and indicates that specific protein-lipid interactions have an ordering effect that reached further than just one additional lipid shell than random protein-lipid interactions (FIG. 35c).

In summary, the structures of an actinoporin pore presented in this work illustrate the intricate interplay between membrane proteins and lipids and provide insights into the unique structural adaptations, ordering effects and functional consequences induced by cholesterol and specific lipid components. These findings contribute to a more comprehensive understanding of the behaviour of membrane proteins in membranes, highlight the important structural and functional role of membrane lipids which is crucial for the application of membrane proteins, e.g. in the design of drugs or protein nanopores for sensing applications.

TABLE 2
X-ray diffraction data collection and
crystallographic refinement statistics.

| Data collection | Fav | |
|---|---|---|
| Wavelength | 1 Å | |
| Resolution range (Å) | 45.8-1.5 | (1.554-1.5) |
| Space group | P 3₂ 2 1 | |
| Unit cell dimensions | | |
| a, b, c (Å) | 61.011 61.011 91.825 | |
| α, β, γ (°) | 90 90 120 | |
| Total reflections | 334684 | (32351) |
| Unique reflections | 32276 | (3196) |
| Multiplicity | 10.4 | (10.1) |
| Completeness (%) | 99.93 | (99.59) |
| Mean I/sigma(I) | 18.73 | (3.25) |
| Wilson B-factor | 11.89 | |
| R_{merge} | 0.09948 | (0.7893) |
| Rₘₑₐₛ | 0.1047 | (0.8317) |
| Rₚᵢₘ | 0.03232 | (0.26) |
| CC_{1/2} | 0.999 | (0.887) |
| CC* | 1 | (0.969) |
| Refinement | | |
| Reflections used in refinement | 32269 | (3195) |
| Reflections used for R-free | 1978 | (194) |
| R-work | 0.1633 | (0.2316) |
| R-free | 0.1853 | (0.2851) |
| CC(work) | 0.969 | (0.915) |
| CC(free) | 0.958 | (0.880) |

| | |
|---|---|
| Number of non-hydrogen atoms | 1780 |
| Protein | 1469 |
| ligands | 23 |
| solvent | 288 |
| Protein residues | 184 |
| RMS(bonds) | 0.008 |
| RMS(angles) | 0.99 |
| Ramachandran favored (%) | 98.35 |
| Ramachandran allowed (%) | 1.65 |
| Ramachandran outliers (%) | 0.00 |
| Rotamer outliers (%) | 0.00 |
| Clashscore | 3.78 |
| Average B-factor | 13.50 |
| macromolecules | 11.23 |
| ligands | 18.04 |
| solvent | 24.68 |

One crystal was used to collect the data. There was one molecule of Fav in the asymmetric unit. Values in parentheses are for the highest-resolution shell.

TABLE 3
Interaction surface of Fav protomer from a pore
prepared on POPG:SM:Cholesterol (1:1:1) membranes
and protomer with removed transmembrane helix
with lipids observed in the structure.

| | interaction surface Å² | |
|---|---|---|
| Lipid | whole protomer | protomer w/o TM-helix |
| L1 | 325.1 | 229.9 |
| L1(B) | 276.9 | 192.9 |
| L2 | 261.9 | 261.9 |
| L3 | 252.5 | 252.5 |
| L4 | 150.9 | 150.9 |
| L4(B) | 38.0 | 38.0 |
| L5 | 163.7 | 163.7 |
| L6 | 402.9 | 193.9 |
| L6(B) | 93.5 | n.a. |
| L7 | 53.9 | 53.9 |
| L8 | 143.6 | 143.6 |
| L9 | 3.9 | 3.9 |
| L10 | 84.9 | 84.9 |
| L11 | 0 | 0 |
| CHOL1 | 144.4 | 108.9 |
| CHOL2 | 131.5 | 83.0 |
| CHOL3 | 152.2 | 69.4 |
| CHOL4 | 74.7 | 74.7 |
| Total lipid | 2759.5 | 2106 |
| Total available | 9339 | 7443 |

TABLE 4
Data acquisition and refinement statistics.

| | | | | |
|---|---|---|---|---|
| Lipid | | | DOPC:SM:CHOL | |
| composition | DOPC:SM | DOPC:SM:CHOL | nanodiscs | POPG:SM:CHOL |

Data Collection

| | | | | |
|---|---|---|---|---|
| Microscope | Krios | Glacios | Glacios | Glacios |
| Magnification | 130,000 | 150,000 | 150,000 | 150,000 |
| Voltage (kV) | 300 | 200 | 200 | 200 |
| Total electron | 40 | 40 | 40 | 40 |
| dose (e−/Å²) | | | | |
| Defocus range (μm) | −1.0~−2.9 | −0.6~−2.8 | −0.6~−2.6 | −0.6~−2.6 |
| Pixel size (Å) | 1.047 | 0.946 | 0.946 | 0.946 |
| Number of | 2,140 | 7.805 | 7,533 | 11,470 |
| micrographs | | | | |
| Data processing | | | | |
| Initial particle | 802,336 | 1,266,343 | 954,368 | 2,705,265 |
| number | | | | |
| Final particle | 174,291 | 223,527 | 193,745 | 118,851 |
| number | | | | |
| Symmetry imposed | C8 | C8 | C8 | C8 |
| Global map | 2.56 | 2.94 | 3.61 | 2.86 |
| resolution | | | | |
| (FSC at 0.143, Å) | | | | |
| EMDB ID | EMD-50057 | EMD-50058 | EMD-50060 | EMD-50059 |
| Model Building | | | | |
| Associated PDB ID | 9EYM | 9EYN | | 9EYO |
| FSCmap-to-model | 2.66 | 3.06 | | 2.98 |
| (0.143)/Å | | | | |
| Composition | | | | |
| Protein chains | 8 | 8 | | 8 |
| Atoms | 13664 | 15296 | | 16160 |
| Protein residues | 1448 | 1448 | | 1456 |
| Ligands | 48 (SFM) | 32 (CLR), | | 32 (CLR), |
| | | 72 (SFM) | | 90 (SFM) |
| Bonds (R.M.S.D.) | | | | |
| Length (Å) | 0.012 (16) | 0.012 (16) | | 0.012(0) |
| Angles (°) | 1.830 (24) | 1.836 (48) | | 1.884 (48) |
| B-factors | | | | |
| (min/max/mean) | | | | |
| Protein residues | 77.29/226.90/ | 136.80/238.01/ | | 73.19/195.42/ |
| | 124.57 | 179.18 | | 101.35 |
| Ramachandran plot (%) | | | | |
| Outliers | 0.00 | 0.00 | | 0.00 |
| Allowed | 1.68 | 1.68 | | 1.67 |
| Favored | 98.32 | 98.32 | | 98.33 |
| Rotamer outliers (%) | 0.68 | 0.68 | | 2.72 |
| Cβ outliers (%) | 0.00 | 0.00 | | 0.00 |
| MolProbity score | 1.01 | 0.89 | | 0.97 |
| Clash Score | 2.30 | 1.47 | | 0.73 |

### Methods

### Protein Expression and Purification

Genes for all three Fav variants (wtFav, ΔN53Fav and ΔN75Fav with mutations R203D and D215N) were cloned into a modified pET28a plasmid, with a His6-tag followed by TEV-cleavage site preceding the N-terminus of the target protein. Transformed E. coli BL21(DE3) cells were grown at 37° C. in Terrific Broth (TB), and gene expression was induced with 0.4 mM isopropyl β-D-1-thiogalactopyranoside when A₆₀₀ reached around 0.7. After induction, the temperature was lowered to 20° C. Cells were harvested after 16 hours by centrifugation at 6,000 g for 5 min, followed by sonication in PBS buffer pH 7.4 (1.8 mM KH₂PO₄, 140 mM NaCl, 10.1 mM Na₂HPO₄, 2.7 mM KCl). The cell lysate was centrifuged at 50,000 g, 4° C. for 45 min. The supernatant was filtered using a 0.22 μm syringe filter and loaded onto a NiNTA 10/50 column (Qiagen) and proteins were eluted with an increasing gradient of imidazole. The haemolytic fractions were pooled and incubated over night with addition of TEV protease with a final concentration of 1% (w/w) at 20° C. After imidazole was removed with dialysis, the protein was again loaded onto a NiNTA 10/50 column (Qiagen) and subsequently subjected to size exclusion chromatography using Superdex 200 prep grade column (GE Healthcare, UK) equilibrated with PBS. Protein-containing factions were concentrated with Amicon Ultra Filter Devices 10 kDa cutoff, aliquoted and stored at −70° C.

### Protein Crystallization and Crystal Structure Determination

High-quality ΔN53Fav monomer crystals were obtained by mixing 1 μl of the protein solution with a concentration of 30 mg/ml with 1 μl of the reservoir solution containing 1.8 M Li₂SO₄ using the vapor-diffusion technique in hanging drops. The drop was equilibrated at 20° C. over 0.5 ml of reservoir solution. The rod-like crystals typically appeared within two days. Crystals were frozen in liquid nitrogen, with 20 (v/v) % 2-methyl-2,4-pentanediol as a cryoprotectant. Diffraction data was collected at 100 K and at the wavelength of 1.0 Å at XDR1 Elettra Synchrotron (Trieste, Italy). The diffraction data was processed to 1.33 Å resolution with XDS³¹ (Table 2). The crystal structure was solved using the symmetry of the space group P3221 by molecular replacement (PHASER³²), with the crystal structure of FraC (PDB-ID 3ZWJ) without loops and α-helices as a search model. Initial Δ53 Fav model was constructed with PHENIX³³ Autobuild and refined by iterative cycles of manual model building in Coot³⁴ and phenix.refine³⁵. The crystal structure of Δ53 Fav is missing the first 25 N-terminal residues of Δ53Fav construct. There are regions of continuous electron density than likely correspond to the N-terminal region, but the density was too weak to be interpretable.

### Preparation of Lipid Vesicles

Lipid vesicles were prepared using lipids from Avanti Polar Lipids, USA. CPE, DOPC, POPG, SM, and cholesterol were dissolved in chloroform or methanol and mixed at appropriate molar ratios (1:1 or 1:1:1). Using a rotavapor (Buchi, Switzerland), a thin lipid film was created and left under a high vacuum for 2 hours. The multilamellar vesicles were produced by resuspending the lipid film in PBS buffer and thoroughly vortexed with the aid of 0.5 mm glass beads (Scientific Industries, USA). The vesicles suspension then underwent at least three fast freeze/thaw cycles. Unilamellar vesicles were formed by passing the multilamellar vesicles through a LiposoFast lipid extruder (Avestin, Canada) with polycarbonate membranes with 100 nm pores.

### Pore Preparation

70 μM of the wild-type Fav monomer was incubated with 14 mM large unilamellar vesicles in PBS buffer for 1 h at 37° C. The mixture was solubilized with 1% lauryldimethylamine oxide. Sample was centrifuged for 5 min at 16,000 g at 20° C. to remove precipitated material. Supernatant was 20-fold diluted in buffer A (50 mM Tris, 0.25 mM Brij 35, pH 7.4) and injected to the Resource Q column (Pharmacia Biotech, Sweden) equilibrated with buffer A. Proteins were eluted with a linear gradient of buffer B (50 mM Tris, 1 M NaCl, 0.25 mM Brij 35, pH 7.4). Oligomeric state of proteins in eluted fractions was monitored with Native PAGE.

### Nanodisc Preparation

Plasmid pMSP1E3D1, bearing the gene for membrane scaffold protein expression was a gift from Stephen Sligar (Addgene plasmid #20066) and was expressed as described before³⁶. Shortly, E. coli BL21(DE3) cells transfected with the plasmid were grown in TB medium at 37° C. until A₆₀₀ reached 2.5. Protein production was initiated with the addition of 1 mM isopropyl β-D-1-thiogalactopyranoside. Cells were harvested 3.5 h later, centrifuged at 6000 g and the pellet stored at −80° C. MSP1E3D1 protein was purified using Chelating Sepharose FF (GE Healthcare, UK) and dialyzed against MSP standard buffer (20 mM TRIS-HCl, 100 mM NaCl, 0.5 mM EDTA, pH 7.4). Protein was concentrated with Amicon Ultra-15 (Millipore, USA) to a final concentration 9 mg/ml.

The assembly of lipid nanodisc was carried out as described³⁷ with the following adjustments. A mixture of 5 mg DOPC, 5 mg SM and 2.5 mg cholesterol was dissolved in chloroform and methanol 1:1 (volume) mixture, and then dried in a rotary evaporator Rotavapor R215 (Buchi, Switzerland). A thin lipid film, formed in a round-bottom flask, was then hydrated by the addition of 100 μl of cholate buffer (20 mM Tris, 25 mM Na-cholate, 140 mM NaCl, pH 7.4), and vigorous vortexing and heating using warm tap water. The mixture was then sonicated in a water bath for 15 min. Finally, 250 μl of purified MSP1E3D1 were added and the mixture was incubated on a bench shaker at 4° C. for 3 h. The nanodiscs were self-assembled upon removal of Na-cholate by overnight dialysis against 3 I of PBS buffer. The dyalized sample was purified using size-exclusion chromatography (Superdex 200 10/300, GE Healthcare Life Sciences). Peak fractions, corresponding to the size of nanodiscs, were analyzed with dynamic light scattering with Prometheus Panta (Nanotemper, Germany) to confirm the presence of ~12 nm particles and the homogeneity of the solution. Concentration of nanodiscs was estimated by measuring the concentration of MSP at A₂₈₀. Nanodiscs were used immediately for cryo-TEM experiments.

### Planar Lipid Membranes Experiments

Experiments were performed with the Nanion Technologies Orbit Mini set up using MECA 4 chips (Ionera, Germany). The data were collected with the Elements Data Reader v 3.8.3 (Elements, Italy) software at 20 nA working range, 20 kHz sampling frequency and room temperature. Planar lipid bilayers were prepared with the brushing method, where 10 mg/ml lipid solution in pure octane was used. To achieve pore insertion, a monomeric Fav variant (deletion of 75 amino acids from the N-terminus and R206D, D218N mutations) was applied at 1.6 μg/ml final concentration to DOPC:SM 1:1 or DOPC:SM:CHOL 1:1:1 membranes. For statistical analysis we calculated the mean current and its standard deviation (unfiltered current traces) for the intact membrane and the first three observed insertion steps (smaller than 200 pA). NR ratio represents the ratio between the noise of the specific insertion step (standard deviation, STD) and intact membrane noise (STD)³⁸. Data were analyzed using Axon pCLAMP 11.1 (Molecular Devices, USA) software our own MatLab R2022a (Mathworks, USA) script. Plotted traces were filtered using MatLab lowpass function with cut off frequency of 1000 Hz.

### Cryo-Electron Microscopy Sample Preparation and Data Acquisition

Isolated wild type Fav pores were prepared as described above (FIG. 36d). Samples of wild type Fav pores on vesicles were prepared similarly, excluding the solubilization and purification steps. Instead, the sample was diluted after the incubation so that the final lipid concentration was 2 mM. Wild type Fav pores on nanodiscs were prepared by incubating nanodiscs (0.4 mg/ml) with monomeric Fav (1 mg/mg) for 1 h at 37° C. After incubation, the sample was 20× concentrated with an Amicon Ultra Filter Device (100 kDa cut-off) and 20× diluted with PBS. This step was repeated three times to remove unbound monomers from the sample.

3 μl of each sample (purified pores, pores on vesicles and pores inserted in nanodiscs) was applied to glow-discharged (GloQube® Plus, Quorum, UK) Quantifoil R1.2/1.3 or R2/2 mesh 200 copper holey carbon grid (Quantifoil, Germany), blotted under 100% humidity at 4° C. for 6-8s, and plunged into liquid ethane with a Mark IV Vitrobot (Thermo Fisher Scientific, USA). Micrographs of the wild-type Fav pore prepared on DOPC:SM vesicles were recorded on Titan Krios G2 (Thermo Fisher Scientific, USA) operated at 300 kV with K2 direct electron detector (Gatan, USA) at Diamond Light Source (UK), all other samples were recorded inhouse on Glacios (Thermo Fisher Scientific, USA) with a Falcon 3EC direct electron detector (Thermo Fisher Scientific, USA) and operated at 200 kV using the EPU software (Thermo Fisher Scientific, USA).

### Cryo-Electron Microscopy Data Processing

All steps of data processing for all samples were performed in cryoSPARC v4³⁹ with built-in algorithms. Movies were aligned and dose-weighted with patch motion correction, CTF was estimated with patch CTF. Micrographs with estimated CTF fit estimation above 6 Å were excluded from further analysis. Initially, particles were picked by hand to generate 2D templates for template picking. From this point on the analysis slightly diverged for the three samples but followed similar steps. Workflows for each sample are provided (FIG. 37, 42, 52). Particles were extracted and underwent several rounds of 2D classification. Particles from best classes were used in ab-initio reconstruction. Particles were then further cleaned up by heterogeneous refinement. Particles from the best class were then re-extracted using local motion correction and used in homogeneous refinement and non-uniform refinement with applied C8 symmetry.

### Model Building

Atomic models of protomers were built with the iterative use of Coot³⁴ and Isolde⁴⁰ plugin for ChimeraX⁴¹. Phenix⁴² was used for applying symmetry and restraint calculation for lipid molecules. The crystal structure of ΔN53Fav monomer was used as the starting model. Details of data acquisition and refinement statistics are shown in Table 4.

### Molecular Dynamics Simulations

All-atom models of the protein pore were prepared using the online server CHARMM-GUI⁴³. Two types of lipid bilayers, with and without CHOL, SM and POPC molecules were used, namely in molar ratios POPC:SM 1:1 and POPC:SM:CHOL 1:1:1. The cryo-EM protein pore structure containing 80 predefined SM and 32 CHOL molecules was inserted into the POPC:SM: CHOL membrane and the protein pore containing 48 predefined SM molecules was inserted into the POPC:SM membrane. All systems including pure membranes were electro-neutralized and immersed in 150 mM NaCl aqueous solution within the simulation box of dimensions 145 Å×145 Å×138 Å.

Each system was exposed to minimization and to a long equilibration phase of 200 ns. All simulations were carried out on GPU's with the CUDA version of the NAMD molecular dynamics software suite⁴⁴. The CHARMM36 force field⁴⁵ was used and water was modelled by the TIP3P water model⁴⁶. All production simulations used the NPT ensemble and their length was at least 1 μs. Temperature was held constant at 303.15 K using the Langevin thermostat with a dampening constant of 1 ps−1. The pressure was held constant at 1.0 bar. The cut-off for nonbonded interactions was set to 12 A, electrostatic interactions were calculated using the Particle Mesh Ewald method (PME).

Correlation between displacements of molecules I and J, which are represented by centres of mass of selected atoms (beads), was evaluated through the ensemble average of displacements taking place during the time Δt:

$${C}_{IJ} ( Δ t ) = \frac{\begin{matrix}〈 ( {r}_{I} ( t + Δ t / 2 ) - {r}_{I} ( t - Δ t / 2 ) ) \\ {( {r}_{J} ( t + Δ t / 2 ) - {r}_{J} ( t - Δ t / 2 ) ) 〉}_{t}\end{matrix}}{\begin{matrix}\sqrt{{〈 {( {r}_{J} ( t + Δ t / 2 ) - {r}_{J} ( t - Δ t / 2 ) )}^{2} 〉}_{t}} \\ \sqrt{{〈 {( {r}_{I} ( t + Δ t / 2 ) - {r}_{I} ( t - Δ t / 2 ) )}^{2} 〉}_{t}}\end{matrix}} \qquad Eq . ( 2 )$$

Displacements during the time step Δt=5 ns were used in correlation evaluation.

Further dynamic properties of the lipid molecules were analysed by evaluating lipid lateral diffusion using the Einstein relation⁴⁷ by estimating the slope of the linear fit to the time dependence of the lipid molecules' mean square displacements.

### REFERENCES FOR EXAMPLE 3

1 Almén, M. S., Nordström, K. J. V., Fredriksson, R. & Schiöth, H. B. Mapping the human membrane proteome: a majority of the human membrane proteins can be classified according to function and evolutionary origin. BMC Biology 7, 50, doi:10.1186/1741-7007-7-50 (2009).
2 Overington, J. P., Al-Lazikani, B. & Hopkins, A. L. How many drug targets are there? Nature Reviews Drug Discovery 5, 993-996, doi:10.1038/nrd2199 (2006).
3 Levental, I. & Lyman, E. Regulation of membrane protein structure and function by their lipid nano-environment. Nature Reviews Molecular Cell Biology 24, 107-122, doi:10.1038/s41580-022-00524-4 (2023).
4 Lee, A. G. How lipids affect the activities of integral membrane proteins. Biochimica et Biophysica Acta (BBA)—Biomembranes 1666, 62-87, doi:https://doi.org/10.1016/j.bbamem.2004.05.012 (2004).
5 Barrera, N. P., Zhou, M. & Robinson, C. V. The role of lipids in defining membrane protein interactions: insights from mass spectrometry. Trends Cell Biol 23, 1-8, doi:10.1016/j.tcb.2012.08.007 (2013).
6 Corradi, V. et al. Emerging Diversity in Lipid-Protein Interactions. Chem Rev 119, 5775-5848, doi:10.1021/acs.chemrev.8b00451 (2019).
7 Han, Y. et al. Mechanical activation opens a lipid-lined pore in OSCA ion channels. Nature, doi:10.1038/s41586-024-07256-9 (2024).
8 Guskov, A. et al. Cyanobacterial photosystem II at 2.9-Å resolution and the role of quinones, lipids, channels and chloride. Nature structural & molecular biology 16, 334-342, doi:10.1038/nsmb.1559 (2009).
9 Gupta, K. et al. The role of interfacial lipids in stabilizing membrane protein oligomers. Nature 541, 421-424, doi:10.1038/nature20820 (2017).
10 Cordero-Morales, J. F. & Vásquez, V. How lipids contribute to ion channel function, a fat perspective on direct and indirect interactions. Curr Opin Struct Biol 51, 92-98, doi:10.1016/j.sbi.2018.03.015 (2018).
11 Xu, P. et al. Structural insights into the lipid and ligand regulation of serotonin receptors. Nature 592, 469-473, doi:10.1038/s41586-021-03376-8 (2021).
12 Dawaliby, R. et al. Allosteric regulation of G protein-coupled receptor activity by phospholipids. Nature chemical biology 12, 35-39, doi:10.1038/nchembio.1960 (2016).
13 Ansell, T. B. et al. LipIDens: simulation assisted interpretation of lipid densities in cryo-EM structures of membrane proteins. Nature communications 14, 7774, doi:10.1038/s41467-023-43392-y (2023).
14 Sharma, K. D., Heberle, F. A. & Waxham, M. N. Visualizing lipid membrane structure with cryo-EM: past, present, and future. Emerging Topics in Life Sciences 7, 55-65, doi:10.1042/etls20220090 (2023).
15 Biou, V. Lipid-membrane protein interaction visualised by cryo-EM: A review. Biochimica et Biophysica Acta (BBA)—Biomembranes 1865, 184068, doi:https://doi.org/10.1016/j.bbamem.2022.184068 (2023).
16 Flores, J. A. et al. Connexin-46/50 in a dynamic lipid environment resolved by CryoEM at 1.9 Å. Nature communications 11, 4331, doi:10.1038/s41467-020-18120-5 (2020).
17 Zhao, P. et al. Structure and activation mechanism of the hexameric plasma membrane H+-ATPase. Nature communications 12, 6439, doi:10.1038/s41467-021-26782-y (2021).
18 Qi, C., Di Minin, G., Vercellino, I., Wutz, A. & Korkhov, V. M. Structural basis of sterol recognition by human hedgehog receptor PTCH1. Science Advances 5, eaaw6490, doi:doi:10.1126/sciadv.aaw6490 (2019).
19 Nadezhdin, K. D. et al. Structural mechanism of heat-induced opening of a temperature-sensitive TRP channel. Nature structural & molecular biology 28, 564-572, doi:10.1038/s41594-021-00615-4 (2021).
20 Sun, C., Zhu, H., Clark, S. & Gouaux, E. Cryo-EM structures reveal native GABA(A) receptor assemblies and pharmacology. Nature 622, 195-201, doi:10.1038/s41586-023-06556-w (2023).
21 Tanaka, K., Caaveiro, J. M., Morante, K., Gonzalez-Manas, J. M. & Tsumoto, K. Structural basis for self-assembly of a cytolytic pore lined by protein and lipid. Nature communications 6, 6337, doi:10.1038/ncomms7337 (2015).
22 Waterhouse, A. et al. SWISS-MODEL: homology modelling of protein structures and complexes. Nucleic acids research 46, W296-W303, doi:10.1093/nar/gky427 (2018).
23 Athanasiadis, A., Anderluh, G., Macek, P. & Turk, D. Crystal structure of the soluble form of equinatoxin II, a pore-forming toxin from the sea anemone Actinia equina. Structure 9, 341-346 (2001).
24 Barlič, A. et al. Lipid phase coexistence favors membrane insertion of equinatoxin-II, a pore-forming toxin from Actinia equina. Journal of Biological Chemistry 279, 34209-34216, doi:10.1074/jbc.M313817200 (2004).
25 Bakrac, B. et al. Molecular determinants of sphingomyelin specificity of a eukaryotic pore-forming toxin. J Biol Chem 283, 18665-18677, doi:10.1074/jbc.M708747200 (2008).
26 Bakrac, B. & Anderluh, G. Molecular mechanism of sphingomyelin-specific membrane binding and pore formation by actinoporins. Advances in experimental medicine and biology 677, 106-115 (2010).
27 García-Montoya, C. et al. Sticholysin recognition of ceramide-phosphoethanolamine. Arch Biochem Biophys 742, 109623, doi:10.1016/j.abb.2023.109623 (2023).
28 Šolinc, G. et al. Pore-forming moss protein bryoporin is structurally and mechanistically related to actinoporins from evolutionarily distant cnidarians. Journal of Biological Chemistry, doi:doi.org/10.1016/j.jbc.2022.102455 (2022).
29 Lemmon, M. A. Membrane recognition by phospholipid-binding domains. Nature reviews. Molecular cell biology 9, 99-111, doi:10.1038/nrm2328 (2008).
30 Moravcevic, K., Oxley, C. L. & Lemmon, M. A. Conditional peripheral membrane proteins: facing up to limited specificity. Structure 20, 15-27, doi:10.1016/j.str.2011.11.012 (2012).
31 Kabsch, W. Integration, scaling, space-group assignment and post-refinement. Acta crystallographica. Section D, Biological crystallography 66, 133-144, doi:10.1107/S0907444909047374 (2010).
32 McCoy, A. J. et al. Phaser crystallographic software. Journal of applied crystallography 40, 658-674, doi:10.1107/S0021889807021206 (2007).
33 Terwilliger, T. C. et al. Iterative model building, structure refinement and density modification with the PHENIX AutoBuild wizard. Acta crystallographica. Section D, Biological crystallography 64, 61-69, doi:10.1107/S090744490705024X (2008).
34 Emsley, P. & Cowtan, K. Coot: model-building tools for molecular graphics. Acta crystallographica. Section D, Biological crystallography 60, 2126-2132, doi:10.1107/S0907444904019158 (2004).
35 Afonine, P. V. et al. Towards automated crystallographic structure refinement with phenix.refine. Acta crystallographica. Section D, Biological crystallography 68, 352-367, doi:10.1107/S0907444912001308 (2012).
36 Bayburt, T. H., Grinkova, Y. V. & Sligar, S. G. Self-Assembly of Discoidal Phospholipid Bilayer Nanoparticles with Membrane Scaffold Proteins. Nano letters 2, 853-856, doi:10.1021/n1025623k (2002).
37 Bayburt, T. H. & Sligar, S. G. Membrane protein assembly into Nanodiscs. FEBS Lett 584, 1721-1727, doi:10.1016/j.febslet.2009.10.024 (2010).
38 Antonini, V. et al. Functional characterization of sticholysin I and W111C mutant reveals the sequence of the actinoporin's pore assembly. PLoS ONE 9, e110824, doi:10.1371/journal.pone.0110824 (2014).
39 Punjani, A., Rubinstein, J. L., Fleet, D. J. & Brubaker, M. A. cryoSPARC: algorithms for rapid unsupervised cryo-EM structure determination. Nature methods 14, 290-296, doi:10.1038/nmeth.4169 (2017).
40 Croll, T. ISOLDE: a physically realistic environment for model building into low-resolution electron-density maps. Acta Crystallographica Section D 74, 519-530, doi:doi:10.1107/S2059798318002425 (2018).
41 Goddard, T. D. et al. UCSF ChimeraX: Meeting modern challenges in visualization and analysis. Protein science: a publication of the Protein Society 27, 14-25, doi:10.1002/pro.3235 (2018).
42 Liebschner, D. et al. Macromolecular structure determination using X-rays, neutrons and electrons: recent developments in Phenix. Acta Crystallogr D Struct Biol 75, 861-877, doi:10.1107/s2059798319011471 (2019).
43 Lee, J. et al. CHARMM-GUI Input Generator for NAMD, GROMACS, AMBER, OpenMM, and CHARMM/OpenMM Simulations Using the CHARMM36 Additive Force Field. J Chem Theory Comput 12, 405-413, doi:10.1021/acs.jctc.5b00935 (2016).
44 Phillips, J. C. et al. Scalable molecular dynamics on CPU and GPU architectures with NAMD. The Journal of chemical physics 153, 044130, doi:10.1063/5.0014475 (2020).
45 Klauda, J. B. et al. Update of the CHARMM All-Atom Additive Force Field for Lipids: Validation on Six Lipid Types. The Journal of Physical Chemistry B 114, 7830-7843, doi:10.1021/jp101759q (2010).
46 Jorgensen, W. L., Chandrasekhar, J., madura, J. D., impey, R. W. & Klein, M. L. Comparison of simple potential functions for simulating liquid water. Journal of Chemical Physics 79, 926-935 (1983).
47 Zidar, J. et al. Liquid-ordered phase formation in cholesterol/sphingomyelin bilayers: all-atom molecular dynamics simulations. The journal of physical chemistry. B 113, 15795-15802, doi:10.1021/jp907138h (2009).

1. An actinoporin monomer comprising a variant of SEQ ID NO: 1 having at least about 60% identity to the sequence of SEQ ID NO: 1 over its entire length.

2. An actinoporin monomer according to claim 1, wherein the variant comprises one or more modifications which (a) improve the ability of an actinoporin pore formed from the monomer to interact with a target analyte, (b) alter the number of actinoporin monomers which form an actinoporin pore or (c) facilitate insertion of the actinoporin monomer or an actinoporin pore formed from the actinoporin monomer into an artificial membrane.

3. An actinoporin monomer according to claim 1, wherein the variant comprises a modification at one or more of the positions corresponding to E67, N68, K73, R74, R77, I91, D99, V100, L101, D215, L252 and E254 in SEQ ID NO: 1.

4. An actinoporin monomer according to claim 1, wherein the variant comprises a modification at the position corresponding to D203 in SEQ ID NO: 1.

5. An actinoporin monomer according to claim 3, wherein D or E is substituted with R, Y, F, N, S or A.

6. An actinoporin monomer according to claim 1, wherein the variant is a fragment lacking one or more amino acids from the N-terminus of SEQ ID NO: 1.

7. (canceled)

8. An actinoporin monomer according to claim 1, wherein the variant is a fragment lacking one or more amino acids from the C-terminus of SEQ ID NO: 1.

9. An actinoporin monomer comprising a variant of SEQ ID NO: 2 having at least about 54% identity to the sequence of SEQ ID NO: 2 over its entire length.

10. An actinoporin monomer according to claim 9, wherein the variant comprises one or more modifications which (a) improve the ability of an actinoporin pore formed from the monomer to interact with a target analyte, (b) alter the number of actinoporin monomers which form an actinoporin pore or (c) facilitate insertion of the actinoporin monomer or an actinoporin pore formed from the actinoporin monomer into an artificial membrane; a modification at one or more of the positions corresponding to E67, N68, K73, R74, R77, I91, D99, V100, L101, D215, L252 and E254 in SEQ ID NO: 1: a modification at the position corresponding to D203 in SEQ ID NO: 1: a substitution of D or E with R, Y, F, N, S or A: a fragment lacking one or more amino acids from the N-terminus of SEQ ID NO: 1: or a fragment lacking one or more amino acids from the C-terminus of SEQ ID NO: 1.

11. A construct comprising two or more covalently attached actinoporin monomers according to claim 1.

12. A polynucleotide which encodes an actinoporin monomer according to claim 1.

13. An actinoporin pore comprising at least one actinoporin monomer according claim 1.

14. An artificial membrane comprising an actinoporin pore derived from Orbicella faveolata.

15. (canceled)

16. A method of determining the presence, absence or one or more characteristics of a target analyte, comprising (a) contacting the target analyte with an actinoporin pore according to claim 13 and (b) taking one or more measurements as the target analyte moves with respect to the actinoporin pore and thereby determining the presence, absence or one or more characteristics of the target analyte.

17. A method according to claim 16, wherein the target analyte is a target polypeptide or target protein.

18-19. (canceled)

20. A kit for characterising a target analyte comprising (a) an actinoporin monomer according to claim 1 and (b) the components of a membrane and/or an analyte binding protein.

21. An apparatus for characterising a target analyte in a sample, comprising (a) a plurality of actinoporin monomers according to claim 1 and (b) a plurality of analyte binding proteins.

22. An array comprising a plurality of membranes according to claim 14.

23. A system comprising (a) a membrane according to claim 14, (b) means for applying a potential across the membrane(s) and (c) means for detecting electrical or optical signals across the membrane(s).

24. (canceled)

25. An apparatus comprising the artificial membrane according to claim 14, wherein the artificial membrane is an in vitro membrane and the actinoporin pore derived from Orbicella faveolata is inserted into the in vitro membrane.


SEQUENCE LISTING (2 sequences)

SEQ ID NO: 1 - protein, 259 aa, Orbicella faveolata
SAPIKANDPNGEVLEEMPKTKRGAEALLADVGLAHFPEMVPNRQELRALLKRSHNAEAIPQEPMDLENLDSEKRAARIAAGTIIAGAELTIGLLQNLLDVLANVNRKCAVGVDNESGFRWQEGSTYFFSGTADENLPYSVSDGYAVLYGPRKTNGPVATGVVGVLAYYIPSIGKTLAVMWSVPFDYNFYQNWWNAKLYSGNQDADYDHYVDLYYDANPFKANGWHERSLGSGLKFCGSMSSSGQATLEIHVLKESETCM

SEQ ID NO: 2 - protein, 183 aa, synthetic construct
RIAAGTIIAGAELTIGLLQNLLDVLANVNRKCAVGVDNESGFRWQEGSTYFFSGTADENLPYSVSDGYAVLYGPRKTNGPVATGVVGVLAYYIPSIGKTLAVMWSVPFDYNFYQNWWNAKLYSGNQDADYDHYVDLYYDANPFKANGWHERSLGSGLKFCGSMSSSGQATLEIHVLKESETCM